(19) United States
(12) Patent Application Publication
(10) Pub. No.: US 2013/0338106 A1
(43) Pub. Date: Dec. 19, 2013

(54) COMPOUNDS INHIBITING LEUCINE-RICH REPEAT KINASE ENZYME ACTIVITY

(71) Applicants: John A. McCauley, Maple Glen, PA (US); Thomas J. Greshock, Collegeville, PA (US); John Sanders, Collegeville, PA (US); Heather H. Stevenson, Conshohocken, PA (US); Jonathan T. Kern, Souderton, PA (US); Ronald K. Chang, Oreland, PA (US)

(72) Inventors: John A. McCauley, Maple Glen, PA (US); Thomas J. Greshock, Collegeville, PA (US); John Sanders, Collegeville, PA (US); Heather H. Stevenson, Conshohocken, PA (US); Jonathan T. Kern, Souderton, PA (US); Ronald K. Chang, Oreland, PA (US)

(21) Appl. No.: 14/000,982

(22) PCT Filed: Feb. 23, 2012
(86) PCT No.: PCT/US12/26219

§ 371 Date: Aug. 22, 2013

Related U.S. Application Data
(60) Provisional application No. 61/447,355, filed on Feb. 28, 2011.

(51) Int. Cl. C07D 409/14; C07D 417/14; C07F 7/10; C07D 409/04

(52) U.S. Cl. CPC C07D 409/14; C07D 409/04; C07D 417/14; C07F 7/10
USPC 514/63; 548/364.4; 514/406; 548/136; 514/363; 548/110; 514/210.2; 544/371; 514/254.07; 544/140; 514/233.5; 546/275.4; 514/337; 546/202; 514/324

(57) ABSTRACT

Disclosed are compounds of Formula I: and the pharmaceutically acceptable salts thereof, wherein “A” is S—; —SO—, —SO₂—, —O— or NR^{a-}, and R^{a}, and R¹ through R⁵ are defined herein. Also disclosed are pharmaceutical formulations comprising a compound of Formula I and methods of treating, managing, or ameliorating diseases amenable to treatment, management, or amelioration by inhibition of LRRK2 kinase activity, for example, Parkinson's disease.

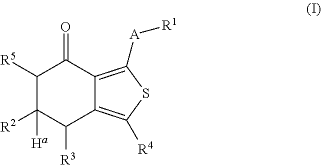

### BACKGROUND

Parkinson's disease (PD) is a movement disorder resulting from progressive loss of dopamine producing neurons. Symptoms associated with Parkinson's disease include motor impairment, bradykinesia, tremor, instability, and other movement related phenotypes. Non-motor symptoms are also associated with the disease, and may include cognitive dysfunction, autonomic dysfunction, and sleep disruption. The combined motor and non-motor symptoms of Parkinson's disease severely impact patient quality of life.

The etiology of Parkinson's disease is not well known. The majority of Parkinson's cases are idiopathic. Recent studies have linked multiple mutations within the Leucine-Rich Repeat Kinase gene with familial forms of Parkinson's disease. Leucine-Rich Repeat Kinase 2 [LRRK2] is a multidomain protein containing kinase and GTPase enzymatic activities. See for example: Aasly et al., Annals of Neurology, Vol. 57(5), May 2005, pp. 762-765; Adams et al., Brain, Vol. 128, 2005, pp. 2777-85; Gilks et al., Lancet, Vol. 365, Jan. 29, 2005, pp. 415-416, Nichols et al., Lancet, Vol. 365, Jan. 29, 2005, pp. 410-412, and U. Kumari and E. Tan, FEBS journal 276 (2009) pp. 6455-6463.

Identification of specific underlying mutations associated with genetic forms of Parkinson's disease has permitted investigation into the effects of mutations in LRRK2 on the disease. These studies suggest that mutations in LRRK2 play a role in the pathogenic pathway of both genetic and sporadic occurrence of Parkinson's disease. See for example, Smith et al., Proc. Natl. Acad. Sci. U.S.A. 102 (51): pp. 18676-81.

Recently, it has been suggested that therapeutic efficacy in addressing Parkinson's disease may be provided by inhibition of LRRK2 Kinase activity with a small molecule inhibitor (See V. Anand and S. Braithwaite, FEBS Journal, 276 (2009) pp. 6428-6435 and references therein).

Currently there is a paucity of compounds known which functionally inhibit LRRK2 kinase function or have specificity for this target, thus, the provision of compounds having LRRK2 inhibiting properties remains an area of unmet medical need. For Example, compounds of Formula Z,

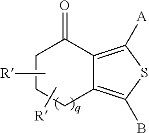

wherein “A” is —S—R*, —SO—R*, —SO₂—R*, and —N(R*)₂ (where “q” is 1 or 2 and R* and R′ are independently —H, -alkyl, substituted alkyl and others) have been described in published international application No. WO98/18792, published May 7, 1998, and investigated for their usefulness as GABA_{A}-receptor inhibitors, however, these compounds have not heretofore been know to exhibit a binding affinity for LRRK2 receptor sites. There remains a need for developing and identifying compounds which have potency in inhibiting LRRK2 kinase functionality and pharmaceutical formulations comprising the same.

### SUMMARY OF THE INVENTION

The invention is directed at pharmaceutical formulations containing compounds, and novel compounds, which, when evaluated in accordance with the LRRK2 enzyme affinity assay methods described herein below, have high affinity, with high kinome specificity, for inhibiting the kinase activity associated with Leucine-Rich Repeat Kinase 2 (inhibition of LRRK2 enzyme activity), a multidomain protein containing kinase and GTPase enzymatic activities, and novel compounds which have high affinity for inhibition of LRRK2 enzyme activity. Formulations and compounds of the invention are believed to be useful in providing alleviation, amelioration, inhibition, management, prevention, reduction, or treatment of conditions, symptoms, or disease states which are amenable to being treated, alleviated, ameliorated, inhibited, managed, prevented, reduced or treated by inhibition of LRRK2-kinase activity. The invention is also directed at the use of compounds and formulations of the invention in treating, reducing, managing, preventing, alleviating, ameliorating, inhibiting, and/or treating symptoms, conditions, disease states amenable to being addressed by the inhibition of LRRK2 kinase enzyme activity.

### DETAILED DESCRIPTION OF THE INVENTION

Compounds suitable for use in formulations of the invention, which comprise the core structure of Formula I (below), surprisingly have been found to have high affinity for inhibition of LRRK2 enzyme activity with high kinome specificity when evaluated in accordance with LRRK2 enzyme affinity assay methods described herein below. Accordingly, compounds of the invention and compounds comprising formulations of the invention are believed to be useful in providing treatment, management, alleviation or amelioration of conditions or disease states which can be treated, managed, alleviated or ameliorated by inhibition of LRRK2-kinase activity, for example, Parkinson's disease, and, for example, non-skin cancers which are associated with mutant LRRK2 kinase activity, for example, as described by Saunders-Pullman, R., et al. in Movement Disorders, published by the Movement Disorder Society via Wiley Online Library [wileyonlinelibrary.com] under DOI: 10.1002/mds.23314, May 26, 2010.

In one embodiment the invention provides formulations comprising compounds that inhibit LRRK2 kinase activity, herein termed LRRK2 inhibitors for convenience, which generally have the structure of Formula I:

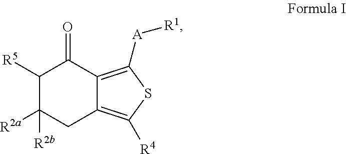

or are provided to the formulation in the form of a pharmaceutically acceptable salt of a compound of Formula I, wherein the substituents “A”, —R¹, R^{2a}, —R^{2b}, —R⁴, and —R⁵ are independently selected for each occurrence, and are further defined below.

In the compounds of the invention and in formulations of the invention:
“A” is —CH₂— and —R¹ is —C₁₋₁₀-alkyl; or
“A” is: —S—; —SO—; —SO₂—; —O—; or —NR^{a}—, wherein —R^{a} is —H, —C₁₋₂₀-alkyl, or —R^{a} is taken together with —R¹ to form a cyclo-amino moiety of the formula:

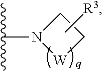

wherein “q” is an integer of 1 to 4, “W” is —CH₂— or —(N—C₁₋₂₀-alkyl)-, —R³ is one or more moieties which are independently: —OH; —C₁₋₆-alkyl; —C₁₋₆-alkoxy,
and wherein, when “A” is not (—CH₂—), —R¹ is:
   (a) —C₁₋₈-alkyl, optionally substituted independently for each occurrence with:
      (i) —(N═N⁺═N⁻); (ii) -halogen, preferably —Cl or —F, more preferably from 1 to 3 —Cl or —F moieties; (iii) —C₁₋₈-alkoxy, optionally substituted; (iv) —OH; (v) morpholinyl-; (vi) pyridyl-; (vii) moieties of the formula (—N(R^{b})₂), where —R^{b} is independently: —H or —C₁₋₁₀-alkyl; (viii) furanyl-; (ix) -aryl, optionally substituted, independently for each occurrence, with: (1) —NR^{c}—(C═O)—CH₃, where —R^{c} is —H or —C₁₋₆-alkyl; (2) -alkoxy; (3) -halogen; (x) -aryloxy; (xi) a moiety of Formula A or Formula B:

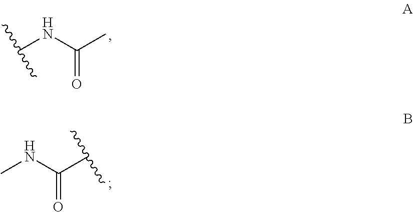

(xii) a moiety of the formula

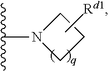

wherein, “q” is an integer of 1 to 4, and wherein independently, for each occurrence, a ring carbon is optionally substituted with —R^{d1}, wherein —R^{d1} is —C₁₋₆-alkyl; (xiii) a moiety of the formula —CH═CHR^{d2}, wherein —R^{d2} is —H or —C₁₋₆-alkyl; (xiv) —Si(R^{e})₃, wherein —R^{e} is a —C₁₋₄-alkyl which is optionally substituted with fluorine, preferably when substituted it is perfluorinated, and more preferably when —R^{e} is prefluorinated alkyl it is a trifluormethyl moiety;
(b) thiopenyl-; (c) heteroaryl-, preferably pyridyl-; (d) furanyl-;
(e) -aryl, optionally substituted, independently for each occurrence with: (i) —N(R^{f})—(C═O)—CH₃, wherein —R^{f} is —H or —C₁₋₆-alkyl; (ii) —C₁₋₂₀-alkoxy; or (iii) -halogen;
(f) a moiety of the formula:

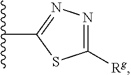

wherein —R^{g} is —C₁₋₆-alkyl or —S—C₁₋₆-alkyl;
—R^{2a} and —R^{2b} are independently —C₁₋₆-alkyl or —H;
—R⁴ is a substituent of the Formula

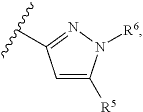

wherein: —R⁶ is:
   (a) —H;
   (b) —C₁₋₆-alkyl;
   (c) —SO₂—C₁₋₆-alkyl;
   (d) —(C═O)—NH—R^{h}, wherein —R^{h} is:
      (I) —(CH₂)₀₋₄—C₆₋₁₀-aryl, the -aryl moiety optionally comprising up to three substituents which are independently for each occurrence: (i) -halogen, if present, preferably —Cl or —F, and preferably bonded meta or para to the ring carbon bonded to the alkyl portion of the moiety, if present, or to the substrate when no alkyl portion is present in the moiety; (ii) —O—C₁₋₆-alkyl; (iii) or (iv) —CN;
      (II) pyridinyl-, optionally substituted, independently for each occurrence, with -halogen;
      (III) piperidinyl-, which is bonded to the substrate through a carbon atom of the ring, and is optionally N-substituted with an acyl moiety, preferably, when present, the N-acyl moiety is —(C═O)—CF₃; or
      (IV) a linear, branched or cyclic alkyl moiety of up to 8 carbon atoms;
   (e) —(C═O)—CH₂—Rⁱ, wherein Rⁱ is: (i) —H; (ii) a para-chlorobenzyl-moiety; (iii) —C₁₋₆-alkyl; or (iv) —C₁₋₂₀-alkoxy; or
   (f) —(C═O)—R^{k}, wherein —R^{k} is: (i) —C₁₋₆-alkyl; (ii) phenyl-, optionally comprising additionally up to 3 substituents which are independently for each occurrence cyano-, halogen- or —C₁₋₆-alkoxy (—O—C₁₋₆-alkyl); or (iii) —(NH)—C₁₋₆-linear alkyl-aryl; and
—R⁵ is: (a) —H; (h) —C₁₋₆-alkyl; or (c)-(CH₂)₀₋₄—C₆₋₁₀-aryl, the -aryl moiety optionally comprising up to three substituents which are independently for each occurrence: (1) -halogen, if present, preferably —Cl or —F, and preferably, the halogen substituent is bonded meta or para to the ring carbon bonded to the alkyl portion of the moiety, if an alkyl portion is present, or bonded meta or para to the ring carbon bonded to the substrate, when no alkyl portion is present in the moiety; (ii) —O—C₁₋₆-alkyl; (iii) —S—C₁₋₆-alkyl or (iv) —CN.

In another embodiment the invention provides a pharmaceutical composition comprising an effective amount of at least one compound of Formula I as the only pharmaceutically active compound. In another embodiment the invention provides a pharmaceutical composition comprising at least one compound of Formula I in combination with an effective amount of at least one other pharmaceutically active ingredient, for example, L-DOPA; dopaminergic agonists such as quinpirole, ropinirole, pramipexole, pergolide and bromocriptine; MAO-B inhibitors such as rasagiline, deprenyl and selegiline; DOPA decarboxylase inhibitors such as carbidopa and benserazide; and COMT inhibitors such as tolcapone and entacapone; or potential therapies such as an adenosine Ata antagonists, metabotropic glutamate receptor 4 modulators, or growth factors such as brain derived neurotrophic factor (BDNF), and a pharmaceutically acceptable carrier.

In one embodiment the invention provides novel LRRK2 enzyme-inhibiting compounds, for Example, the compounds of Formula I, as defined above, with the following provisos: when “A” is —S—, substituents —R⁵ and —R⁶ on the —R⁴ moiety are both —H, and —R^{2a} and —R^{2b} are both -methyl, —R¹ is not the moiety [R^{x1}—CH₂—], wherein —R^{x1} is: —CF₃; —CH₂-phenyl; -phenyl; —H; a moiety of the Formula of Cmpd “X”:

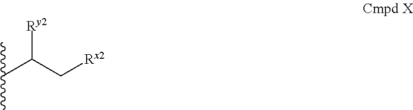

wherein, —R^{x2} and —R^{y2} are selected to give a moiety of Cmpd “X” defining M-1 to M-5, as shown in Table I:

TABLE I

| | Cmpd X | | | | |
|---|---|---|---|---|---|
| | M-1 | M-2 | M-3 | M-4 | M-5 |
| —R^{x2} | —H | —OH | —OCH₃ | —H | -Phenyl |
| —R^{y2} | —H | —H | —H | —CH₃ | —H |

or a moiety of Cmpd “Y”:

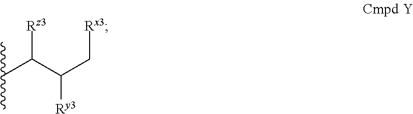

wherein —R^{x3}, —R^{y3}, and —R^{z3} are selected to give a moiety of Cmpd “Y” defining M-1 to M-7, as shown in Table II:

TABLE II

| Cmpd Y | M-1 | M-2 | M-3 | M-4 | M-5 | M-6 | M-7 |
|---|---|---|---|---|---|---|---|
| —R^{x3} | —H | —OH | —H | —OH | —H | —CH₃ | —CH₂—CH₃ |
| —R^{y3} | —H | —H | —OH | —OH | —CH₃ | —H | —H |
| —R^{z3} | —H | —H | —H | —H | —H | —H | —H; |

or, when “A” is —N—, —R⁵ and —R⁶ on the —R⁴ substituent are both —H, and —R^{2a} and —R^{2b} are both -methyl, —R¹ is not a —C₁₋₄ alkyl moiety; or when “A” is —SO₂— and —R^{2a} and —R^{2b} are both -methyl, —R¹ is not -methyl. In some embodiments wherein “A” is —S—, the moieties —R⁵ and —R⁶ on the —R⁴ substituent are both —H, and —R^{2a} and —R^{2b} are both -methyl, it is preferred that —R¹ is not —C₁₋₆-alkyl.

In another embodiment the invention provides a method of inhibiting LRRK2 Kinase activity (this is to say, inhibiting the kinase activity associated with Leucine-Rich Repeat Kinase 2 [LRRK2], a multidomain protein containing kinase and GTPase enzymatic activities) in a patient in need of therapy for a condition amenable to treatment by such kinase activity inhibition, for example, treatment or prevention of neurologic damage associated with Parkinson's disease, for example, improvement in dopaminergic tone and in providing symptomatic benefit, for example, in treating, alleviating, ameliorating, or managing motor and non-motor symptoms of Parkinson's disease. Such treatment, alleviation, amelioration, or management of a disease state comprises administering to a patient in need thereof an effective amount of one or more compounds of Formula I:

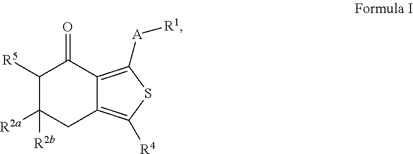

or a pharmaceutically acceptable salt of one or more compounds of Formula I, wherein “A”, —R¹, —R^{2a}, —R^{2b}, —R⁴, and —R⁵ substituents have been defined above.

In another embodiment the invention provides a method for treating symptoms associated with Parkinson's disease in a patient in need of such treatment, said method comprising administering to said patient an effective amount of at least one compound of Formula I, IA, IB, IC, ID, IE, IF or IG, optionally in combination with one or more additional therapeutic agents, for example: L-DOPA; dopaminergic agonists such as quinpirole, ropinirole, pramipexole, pergolide and bromocriptine; MAO-B inhibitors such as rasagiline, deprenyl and selegiline; DOPA decarboxylase inhibitors such as carbidopa and benserazide; and COMT inhibitors such as tolcapone and entacapone; or potential therapies such as an adenosine A2a antagonists, metabotropic glutamate receptor 4 modulators, or growth factors such as brain derived neurotrophic factor (BDNF), and a pharmaceutically acceptable carrier.

As used herein, unless otherwise specified, the term “LRRK2 inhibitor” means a compound of the invention exhibiting a potency (IC₅₀) of less than about 5000 nM when assayed in accordance with the LRRK2 G2019S LanthaScreen® assay described herein below. Preferred compounds exhibit are at least 100-fold selectivity for 90% or more of the kinase enzymes tested using a Caliper LifeSciences' ProfilerPro Kinase Selectivity Assay Kits assay described herein.

As described herein, unless otherwise indicated, the use of a compound in treatment means that an amount of the compound, generally contained within a formulation that comprises other excipients, is administered in aliquots of an amount, and at time intervals, providing at least a therapeutic serum level of the compound over the interval between dose administration.

As used herein, unless otherwise specified, the following terms have the following meanings:

“at least one”, whether used in reference to the number of optional substituents or in reference to compositions comprising “at least one compound of Formula I” or “at least one pharmaceutical excipient” means that one member of the selection group is present, and more than one may additionally be present, up to either the number of constituents enumerated, or, where no upper limit is enumerated, in the case of substituents on a compound, up to all available bonding positions being occupied by the class of substituents; typically, if present, for constituents, up to about 6 constituents are present, typically, if present, preferably from 1 to about 4 of the enumerated substituents are present; “at least one” means that one, or more than one, substituent is present on a moiety, or compound, or excipient is contained in a composition, and when referring to compositions, the constituent is present at a purity level consistent with acceptable pharmaceutical practice, although amounts of more than one isolated compound, for example, 2, 3, 4, 5, or 6 different compounds, or more than one isolated excipient, for example 2, 3, 4, 5, or 6 different excipients, can be combined in providing a suitable composition; whether used in reference to substituents or constituents of a composition, “one or more”, means the same as “at least one”;

“concurrently” and “contemporaneously” both include in their meaning (1) simultaneously in time (e.g., at the same time); and (2) at different times but within the course of a common treatment schedule;

“consecutively” means one following the other;

“sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component;

“effective amount” or “therapeutically effective amount” is meant to describe the provision of an amount of compound or of a composition comprising a compound of the present invention which is effective in treating or inhibiting the diseases or conditions described herein, and thus producing the desired therapeutic, ameliorative, inhibitory or preventative effect; thus, for example, in the methods of treating or preventing symptoms associated with Parkinson's disease, as described herein “effective amount” (or “therapeutically effective amount”) means, for example, the amount of a compound of Formula I that results in therapeutic response of a condition or disease state, including management, alleviation, amelioration, treatment of the disease or alleviation, amelioration, reduction, or disappearance of one or more symptoms attributed to the disease state and/or long-term disease stabilization, for example, as may be determined by the analysis of pharmacodynamic markers or clinical evaluation of patients afflicted with the disease;

“patient” and “subject” means an animal, such as a mammal (e.g., a human being, and is preferably a human being);

“prodrug” means compounds that are rapidly transformed, for example, by hydrolysis in blood, in vivo to the parent compound, e.g., to a compound of Formulae I, IA, or IB described herein, or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes prodrugs of the novel compounds of this invention;

“solvate” means a physical association of a compound of this invention with one or more solvent molecules; this physical association involves varying degrees of ionic and covalent bonding, including hydrogen bonding; in certain instances the solvate will be capable of isolation, for example when one or more solvent molecules are incorporated in the crystal lattice of the crystalline solid; “solvate” encompasses both solution-phase and isolatable solvates; non-limiting examples of suitable solvates include ethanolates, methanolates, and the like; “hydrate” is a solvate wherein the solvent molecule is H₂O.

As used herein, unless otherwise specified, the following terms used to describe moieties, whether comprising the entire definition of a variable portion of a structural representation of a compound or a substituent appended to a variable portion of a structural representation have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, the chemical term “acyl”, defined below, is equivalently described herein by the term itself, or by typographical representations “R′—(C═O)—” or “R′—C(O)—”, or by the structural representation:

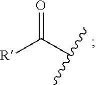

“acyl” means an R′—C(O)—, where R′ is linear, branched or cyclic alkyl; linear, branched or cyclic alkenyl, linear, branched or cyclic alkynyl, each of which moieties can be substituted; the substituent is bonded through the carbonyl carbon; non-limiting examples of suitable acyl groups include formyl, acetyl, propanoyl, 2-methylpropanoyl, butanoyl and cyclohexanoyl;

“alkenyl” means an aliphatic hydrocarbon moiety which is not aromatic but includes in its structure at least one constituent of the structure —(R′C═CR′₂) or —(R′C═CR′)-, where R′ is a defined substituent, for example —H or -alkyl; the alkenyl moiety can be incorporated into a linear hydrocarbon chain, or incorporated into a cyclic hydrocarbon chain (termed “cycloalkenyl”) and can comprise further, linear, branched, or cyclic substituents depending from the carbon atoms of the chain, preferably the chain comprises about 2 to about 15 carbon atoms; more preferably from about 2 to about 12 carbon atoms; and more preferably chains comprise from about 2 to about 6 carbon atoms; the term “substituted alkenyl”, unless specified otherwise by a recitation of specific substituents defining the term, means that the alkenyl group is substituted by one or more substituents which are independently for each occurrence: halo, alkyl, aryl, cycloalkyl, cyano, alkoxy and —S(alkyl);

“alkoxy” means a moiety of the structure: alkyl-O— (i.e., the bond to the substrate moiety is through the ether oxygen), wherein the alkyl portion of the moiety is as defined below for alkyl; non-limiting examples of suitable alkoxy groups include methoxy, ethoxy, n-propoxy, isopropoxy, n-butoxy and heptoxy;

“alkoxycarbonyl” means a moiety of the structure alkyl-O—C(O)—, equivalently represented as [alkyl-O—(C═O)—] and also as R—O(C═O)—, where “R” is a defined alkyl moiety, (i.e., the bond to the parent moiety is through the carbonyl carbon) wherein the alkyoxy portion of the moiety is as previously defined; non-limiting examples of suitable alkoxycarbonyl groups include methoxycarbonyl and ethoxycarbonyl;

“alkyl” (including the alkyl portions of other moieties, such as trifluoromethyl-alkyl- and alkoxy-) means an aliphatic hydrocarbon chain comprising from about 1 to about 20 carbon atoms (that is, “C₁₋₂₀ alkyl”), preferably 1 to about 10 carbon atoms (herein “C₁₋₁₀ alkyl”), unless the term is modified by an indication that a shorter chain is contemplated, for example, an alkyl moiety of up to 8 carbon atoms (designated herein “C₁₋₈ alkyl”); the term “alkyl”, unless specifically limited by another term, for example, “linear”, “branched”, or “cyclic”, includes alkyl moieties which are linear (a hydrocarbon chain with no aliphatic hydrocarbon “branches” appended to it); branched (a main hydrocarbon chain comprising up to the maximum specified number of carbon atoms with a lower-alkyl chain appended to one or more carbon atoms comprising, but not terminating, the main hydrocarbon chain); and cyclic (the main hydrocarbon chain forms an cyclic aliphatic moiety of from 3 carbon atoms, the minimum number necessary to provide a cyclic moiety, up to the maximum number of specified carbon atoms), accordingly when unmodified, the term “C_{1-X} alkyl” refers to linear, branched, or cyclic alkyl, and the “C_{1-X}” designation means: for a cyclic moiety a ring comprising at minimum 3 carbon atoms up to “X” carbon atoms; for a branched moiety, a main chain of at least 3 carbon atoms up to “X” carbon atoms with at least one linear or branched alkyl moiety bonded to a carbon atom which does not terminate the chain; and for a linear alkyl, a moiety comprising one carbon atom (i.e., -methyl), up to “X” carbon atoms; when the term “alkyl” is modified by “substituted” or “optionally substituted” it means an alkyl group having substituents in accordance with the relevant definitions appearing below; where use of the terms “substituted” or “optionally substituted” modify “alkyl” and substituent moieties are not specifically enumerated, the substituents bonded to the alkyl substrate are independently for each occurrence (in accordance with definitions appearing herein): C₁₋₂₀ alkyl; halogen; -alkoxy; —OH; —CN; alkylthio-; amino, —NH(alkyl), —NH(cycloalkyl), —N(alkyl)₂, —(C═O)-0H; —C(O)O-alkyl; —S(alkyl); or —S(O₂)-alkyl; or -aryl; cycloalkyl moieties may alternatively, or in addition, be substituted with one or more, “ring-system substituents” as that term is defined herein;

“lower alkyl” means a group comprising about 1 to about 6 carbon atoms in the chain (i.e. C₁₋₆); non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl, where the term “alkyl” is indicated with two hyphens (i.e., “-alkyl-” it indicates that the alkyl moiety is bonded in a manner that the alkyl moiety connects a substrate with another moiety, for example, “-alkyl-OH” indicates an alkyl moiety connecting a hydroxyl moiety to a substrate;

“alkylaryl” (or alkaryl) means an alkyl-aryl- group (i.e., the bond to the parent moiety is through the aryl group) wherein the alkyl group is unsubstituted or substituted as defined above, and the aryl group is unsubstituted or substituted as defined below; preferred alkylaryl moieties comprise a lower alkyl group; non-limiting examples of suitable alkylaryl groups include o-tolyl, p-tolyl and xylyl;

“alkylsulfinyl” means an alkyl-S(O)— moiety (i.e., the moiety is bonded to a substrate through the sulfur atom of the sulfinyl moiety); “alkylthio” means an alkyl-S— group (i.e., the moiety is bonded to a substrate through the sulfur atom of the moiety); “alkylsulfonyl” means an alkyl-S(O₂)— group (i.e., the moiety is bonded to a substrate through the sulfur atom of the sulfonyl moiety), suitable alkyl groups can be unsubstituted or substituted as previously defined; preferred groups are those in which the alkyl group is lower alkyl;

“alkynyl” means an aliphatic hydrocarbon group (chain) comprising at least one moiety of the structure:

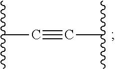 [SMILES: *C#C*]

or the structure:

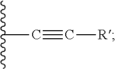

wherein R′ is a defined substituent, the alkynyl moiety can be incorporated into a linear or branched hydrocarbon chain, or incorporated into a cyclic hydrocarbon chain (non-aromatic, termed “cycloalkynyl”,); preferably hydrocarbon chains of an alkynyl moiety comprises about 2 to about 15 carbon atoms; more preferably alkynyl groups comprise about 2 to about 12 carbon atoms in the chain; and more preferably about 2 to about 4 carbon atoms in the chain;

“amino” means an —NR₂ group wherein R is selected independently for each occurrence from —H or alkyl, alkylamino means —NR′₂, wherein one R′ is -alkyl and the other is —H or -alkyl selected independently for each occurrence, non-limiting examples of alkylamino moieties are —NH—CH₃ (methylamino-) and —N(CH₃)₂ (dimethylamino);

“ammonium ion” means —N⁺R_{3′} wherein R is independently —H, alkyl, substituted alkyl, or the cationic portion of a dissociated acid capable of producing an ammonium ion from an amine; when not explicitly shown in representations herein the presence of an ammonium ion presumes that a charge-balancing anion is associated with the ammonium ion moiety, which anion is derived from the anionic portion of the acid used to provide said ammonium ion;

“aryl” (sometimes abbreviated “ar”) means an aromatic monocyclic or multicyclic ring system comprising about 6 to about 14 carbon atoms (denoted herein also as “C₆₋₁₄-aryl”), preferably about 6 to about 10 carbon atoms (“C₆₋₁₀-aryl”); the aryl group can be optionally substituted with one or more independently selected “ring system substituents” (defined below). Non-limiting examples of suitable aryl groups include phenyl

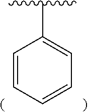

and naphthyl

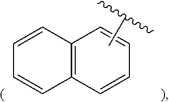

wherein bonding can be through any of the carbons in the aromatic ring, and wherein any ring carbon atoms not participating in a bond to the substrate may have bonded to it a substituent other than —H, independently selected in each instance from the list of “ring-system substituents” defined herein, or as defined in each instance where the term is used in conjunction with an enumerated list of substituents;

“aryloxy” means an aryl-O— group (i.e., the moiety is bonded to a substrate through the ether oxygen) wherein the aryl group is unsubstituted or substituted as defined above; non-limiting examples of suitable aryloxy groups include phenoxy and naphthoxy;

“aryloxycarbonyl” means an aryl-O—C(O)— group (i.e., the bond to a substrate is through the carbonyl carbon) wherein the aryl group is unsubstituted or substituted as previously defined; non-limiting examples of suitable aryloxycarbonyl groups include phenoxycarbonyl and naphthoxycarbonyl;

“arylsulfinyl” means an aryl-S(O)— group, “arylsulfonyl” means an aryl-S(O₂)— group, and “arylthio” means an aryl-S— group (i.e., the bond to the parent moiety is through the sulfur atom in each case) wherein aryl is unsubstituted or substituted as previously defined;

a “carboxylic acid” moiety means a substituent having the formula “—C(O)—OH”, wherein the moiety is bonded to a substrate is through the carbonyl carbon;

“cycloalkyl” defined above with the “alkyl” definition, means a non-aromatic mono- or multicyclic ring system comprising about 3 to about 20 carbon atoms which may be substituted as defined herein; the term includes multicyclic cycloalkyls, for example, 1-decalin, norbornyl, adamantyl and the like;

“halogen” means fluorine, chlorine, bromine, or iodine; preferred halogens are fluorine, chlorine and bromine, a substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group;

“heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination; preferred heteroaryl moieties comprise about 5 to about 6 ring atoms; the “heteroaryl” can be optionally substituted by one or more independently selected “ring system substituents” (defined below); the prefix aza, azo, oxa, oxo, thia or thio before the heteroaryl root name means that at least a nitrogen, oxygen or sulfur atom, respectively, is present as a ring atom, and in some embodiments 2 or more heteroatoms are present in a ring, for example, a pyrazole or a thiazole moiety; a nitrogen atom of a heteroaryl can be optionally oxidized to the corresponding N-oxide; examples of heteroaryl moieties include: pyridyl-,

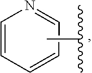

thiopenyl-

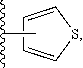

furanyl-,

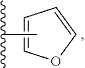

pyrazinyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl, furopyridine, for example:

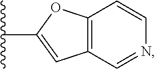 [SMILES: *c1cc2cnccc2o1]

and the like (unless otherwise noted, bonded to the substrate through any available atom that results in a stable bonding arrangement);

“heterocyclyl” (or heterocycloalkyl) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination; there are no adjacent oxygen and/or sulfur atoms present in the ring system; preferred heterocyclyl moieties contain about 5 to about 6 ring atoms; the prefix aza, oxa or thia before the heterocyclyl root name means that at least one nitrogen, oxygen or sulfur atom, respectively, is present as a ring atom; the heterocyclyl can be optionally substituted by one or more independently selected “ring system substituents” (defined below); the nitrogen or sulfur atom of the heterocyclyl can be optionally oxidized to the corresponding N-oxide, S-oxide or S,S-dioxide; non-limiting examples of suitable monocyclic heterocyclyl rings include piperidyl, pyrrolidinyl, piperazinyl, morpholinyl-

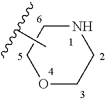

(where unless otherwise noted the moiety is bonded to the substrate through any of ring carbon atoms C2, C3, C5, or C6), thiomorpholinyl, thiazolidinyl, 1,3-dioxolanyl, 1,4-dioxanyl, tetrahydrofuranyl, tetrahydrothiophenyl, tetrahydrothiopyranyl, and the like;

The term “substituted” means that one or more of the enumerated substituents (or, where none are enumerated, the default substituents for the substrate that are specified in the definitions section) can occupy one or more of the bonding positions on the substrate typically occupied by “—H”, provided that such substitution does not exceed the normal valency rules for the atom in the bonding configuration present in the substrate, and that the substitution results in a stable compound, e.g., mutually reactive substituents are not present geminal or vicinal to each other, and wherein such a compound is sufficiently robust to survive isolation to a useful degree of purity from a reaction mixture; when the text indicates optional substitution of a moiety (e.g. “optionally substituted”) the term means “if present, one or more of the enumerated (or default substituents for the specified substrate) can be present on the substrate in a bonding position normally occupied by a hydrogen atom” in accordance with the definition of “substituted” presented herein;

“ring-system substituent” means a substituent attached to an aromatic or non-aromatic ring system that, for example, replaces a bonding position normally occupied by a hydrogen atom on the ring system; unless modified by exclusions or additions, the term “ring-system substituent” means one or more moieties independently selected from: alkyl, aryl, heteroaryl, aralkyl, alkylaryl, aralkenyl, heteroaralkyl, alkylheteroaryl, heteroaralkenyl, hydroxy (also termed “hydroxyl” when standing alone as a substituent moiety), hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, R⁶⁰R⁶⁵N—, R⁶⁰R⁶⁵N-alkyl-, R⁶⁰R⁶⁵NC(O)— and R⁶⁰R⁶⁵NSO₂—, wherein R⁶⁰ and R⁶⁵ are each independently: hydrogen, alkyl, aryl, and aralkyl (as defined herein);

“tetrahydropyranyl” moiety means a 6-member cyclic ether of the formula:

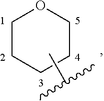

where, the bond line having an open end in the center of the structure and terminated at the other end with a wavy line indicates that the substituent is bonded to the substrate to which it is attached through any of carbon atoms 1 to 5, and wherein any of the bonding positions on carbons 1 to 5 normally occupied by a hydrogen atom, that is, the bonding positions on carbon atoms 1 to 5 which are not occupied by the bond to the substrate can optionally be occupied by specified or optional substituents;

“piperidinyl” means:

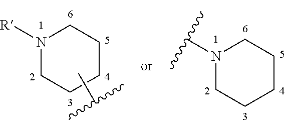

where, the open bond line terminated on one end with a wavy line indicates the ring atom through which the moiety is bonded to the substrate (i.e., any of carbon atoms 2 to 6 (left-hand structure) or the ring nitrogen atom (right-hand structure), and wherein any of the bonding positions on the nitrogen atom or on carbon atoms 2 to 6 not participating in a bond to the substrate and normally occupied by a hydrogen atom can be bonded to a specified or optional substituent, and wherein R′, if present, is either —H or another specified substituent;

“pyridinyl” means:

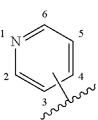

where, the bond-terminated-with-wavy-line indicates that the pyridinyl moiety is bonded to the substrate at any of carbon atoms 2 to 6, and wherein any of the bonding positions on carbons 2 to 6 normally occupied by a hydrogen atom, that is, any position on carbon 2 to 6 which is not the bond to the substrate, can optionally be occupied by a specified substituent;

“quinoline” means:

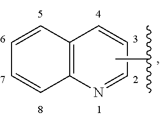

where, the bond-terminated-with-wavy-line indicates that the moiety is bonded to the substrate through any of carbon atoms 2 to 8, and wherein any of the bonding positions on carbon atoms 2 to 8 normally occupied by a hydrogen atom, that is, any bonding positions on carbon atoms 2 to 8 which are not bonded to the substrate, can optionally be occupied by one of a list of enumerated substituents;

for any of the foregoing ring-system moieties, bonding of the moiety through a specific ring carbon atom (or heteroatom) is sometimes described for convenience and “bonded through C—X to C—Y carbon atoms”, where “X” and “Y” are integers referring to the carbon atoms, for example, as numbered in the examples above;

“hydroxyl moiety” and “hydroxy” means an HO— group, “hydroxyalkyl” means a substituent of the formula: “HO-alkyl-”, wherein the alkyl group is bonded to the substrate and may be substituted or unsubstituted as defined above; preferred hydroxyalkyl moieties comprise a lower alkyl; Non-limiting examples of suitable hydroxyalkyl groups include hydroxymethyl and 2-hydroxyethyl; and

bonding sequence is indicated by hyphens where moieties are represented in text, for example -alkyl, indicates a single bond between a substrate and an alkyl moiety, -alkyl-X, indicates that an alkyl group bonds an “X” substituent to a substrate, and in structural representation, bonding sequence is indicated by a wavy line terminating a bond representation, for example:

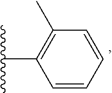 [SMILES: *c1ccccc1C]

indicates that the methylphenyl moiety is bonded to a substrate through a carbon atom ortho to the methyl substituent, while a bond representation terminated with a wavy line and drawn into a structure without any particular indication of a atom to which it is bonded indicates that the moiety may be bonded to a substrate via any of the atoms in the moiety which are available for bonding, for example:

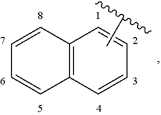

indicates that the naphthalene moiety may be bonded to the substrate through any of carbons 1 to 8.

Any carbon or heteroatom with unsatisfied valences in the text, schemes, examples, structural formulae, and any Tables herein is assumed to have a hydrogen atom or atoms of sufficient number to satisfy the valences.

One or more compounds of the invention may also exist as, or optionally be converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “pharmaceutical composition” as used herein encompasses both the bulk composition and individual dosage units comprised of more than one (e.g., two) pharmaceutically active agents such as, for example, a compound of the present invention and an additional agent as described herein, along with any pharmaceutically inactive excipients. As will be appreciated by the ordinarily skilled artisan, excipients are any constituent which adapts the composition to a particular route of administration or aids the processing of a composition into a dosage form without itself exerting an active pharmaceutical effect. The bulk composition and each individual dosage unit can contain fixed amounts of the afore-said “more than one pharmaceutically active agents”. The bulk composition is material that has not yet been formed into individual dosage units.

Illustrative dosage units include an oral dosage unit, for example, a table, capsule, liquid suitable for imbibing, pills and the like. Similarly, the herein-described methods of treating a patient by administering a pharmaceutical composition of the present invention is also intended to encompass the administration of the afore-said bulk composition and individual dosage units.

This invention also includes the compounds of this invention in isolated and purified form. Polymorphic forms of the compounds of formula A1, and of the salts, solvates and prodrugs of the compounds of formula A1, are intended to be included in the present invention. Certain compounds of the invention may exist in different isomeric (e.g., enantiomers, diastereoisomers, atropisomers) forms. The invention contemplates all such isomers both in pure form and in admixture, including racemic mixtures. Enol forms are also included.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Where diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

Where the compounds of Formula I form salts by known, ordinary methods, these salts are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable salts) are preferred. Salts of the compounds of the Formula I may be formed, for example, by reacting a compound of Formula I with an amount of acid or base, for example, an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al., Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l Union of Pure and Applied Chemistry, pp. 330-331. These disclosures are incorporated herein by reference.

Exemplary acid addition salts include, but are not limited to, acetates, including trifluoroacetate salts, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexyl-amine, choline, tromethamine, and salts with amino acids such as arginine, lysine and the like. Basic nitrogen-containing groups may be converted to an ammonium ion or quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid and base salts are intended to be pharmaceutically acceptable salts within the scope of the invention and all acid and base salts are considered equivalent to the free forms of the corresponding compounds for purposes of the invention.

Compounds of Formula I, and salts, solvates and prodrugs thereof, may exist in their tautomeric form (for example, ketone/enol tautomeric forms or imine-enamine tautomeric forms). All such tautomeric forms are contemplated herein as part of the present invention.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, and in sufficient purity to be characterized by standard analytical techniques described herein or well known to the skilled artisan.

A functional group in a compound termed “protected” means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

Occurrence of a variable (e.g., aryl, heterocycl, R³, etc.) more than once in any moiety or in any compound of Formula I, definition of a variable for each occurrence is independent of its definition at every other occurrence unless specified otherwise.

As used herein, the term “composition” is intended to encompass a product comprising the specified ingredients in the specified amounts, and any product which results, directly or indirectly, from combination of the specified ingredients in the specified amounts.

The present invention also embraces isotopically-labeled compounds of the present invention which are structurally identical to those recited herein, but for the fact that a statistically significant percentage of one or more atoms in that form of the compound are replaced by an atom having an atomic mass or mass number different from the atomic mass or mass number of the most abundant isotope usually found in nature, thus altering the naturally occurring abundance of that isotope present in a compound of the invention. Examples of isotopes that can be preferentially incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detection. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of Formula I can generally be prepared by following procedures analogous to those disclosed in the Schemes and/or in the Examples herein below, by substituting an appropriate isotopically labeled reagent for a non-isotopically labeled reagent.

As mentioned above, this invention provides pharmaceutical formulations, also termed herein, pharmaceutical compositions, for use in the inhibition of LRRK2 kinase activity which comprise an effective amount of at least one compound of Formula I:

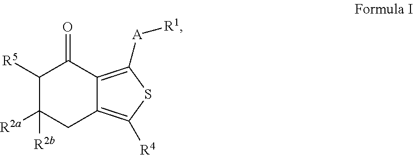

wherein:
“A, —R¹, —R^{2a}, —R^{2b}, —R⁴, and —R⁵ are defined herein and at least one pharmaceutically acceptable excipient, for example, a carrier, as described in detail herein. It will be appreciated that pharmaceutically formulations of the invention may comprise more than one compound of Formula I, for example, the combination of two or three compounds of Formula I, each present by adding to the formulation the desired compound in a pharmaceutically acceptably pure form, in the desired amount.

In some embodiments it is preferred to incorporate into a formulation of the invention at least one compound of Formula I which is a compound of Formula IA:

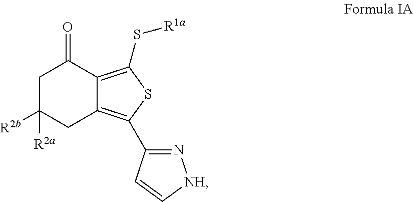

wherein —R^{1a}, —R^{2a}, and —R^{2b} are defined below in Table III.

TABLE III

| Cmpd. No./Nomenclature | —R^{1a} | —R^{2a}/—R^{2b} |
|---|---|---|
| IA-01/3-[(6-Hydroxyhexyl)sulfanyl]-6,6-dimethyl-1-(1H- | —(CH₂)₆—OH | —CH₃/—CH₃ |
| pyrazol-3-yl)-6,7-dihydro-2-benzothiophen-4(5H)-one | | |
| IA-02/3-(Cyclopentylsulfanyl)-6,6-dimethyl-1-(1H-pyrazol-3- [symbol] 6,7-dihydro-2-benzothiophen-4(5H)-one | 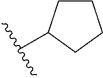 [SMILES: *C1CCCC1] | —CH₃/—CH₃ |
| IA-03/3-(Cyclopentylsulfanyl)-1-(1H-pyrazol-3-yl)-6,7-dihydr [symbol] | ″ | —H/—H |
| 2-benzothiophen-4(5H)-one | | |
| IA-04/6,6-Dimethyl-3-[(2-methylpropyl)sulfanyl]-1-(1H-pyraz [symbol] | —CH₂—CH(—CH₃)₂ | —CH₃/—CH₃ |
| 3-yl)-6,7-dihydro-2-benzothiophen-4(5H)-one | | |
| IA-05/3-[(3-Chloropropyl)sulfanyl]-6,6-dimethyl-1-(1H-pyraz [symbol] | —CH₂—CH₂—CH₂—Cl | —CH₃/—CH₃ |
| 3-yl)-6,7-dihydro-2-benzothiophen-4(5H)-one | | |
| IA-06/3-(Butylsulfanyl)-6,6-dimethyl-1-(1H-pyrazol-3-yl)-6,7- | —(CH₂)₃—CH₃ | —CH₃/—CH₃ |
| dihydro-2-benzothiophen-4(5H)-one | | |
| IA-07/3-[(Cyclopropylmethyl)sulfanyl]-6,6-dimethyl-1-(1H- | —CH₂-cyclopropyl | —CH₃/—CH₃ |
| pyrazol-3-yl)-6,7-dihydro-2-benzothiophen-4(5H)-one | | |
| IA-08/6,6-Dimethyl-3-(propan-2-ylsulfanyl)-1-(1H-pyrazol-3- | —CH(—CH₃)₂ | —CH₃/—CH₃ |
| yl)-6,7-dihydro-2-benzothiophen-4(5H)-one | | |
| IA-09/6,6-Dimethyl-3-(propylsulfanyl)-1-(1H-pyrazol-3-yl)-6, [symbol] | —(CH₂)₂—CH₃ | —CH₃/—CH₃ |
| dihydro-2-benzothiophen-4(5H)-one | | |
| IA-10/3-(Butan-2-ylsulfanyl)-6,6-dimethyl-1-(1H-pyrazol-3-yl [symbol] 6,7-dihydro-2-benzothiophen-4(5H)-one | 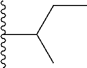 [SMILES: *C(C)CC] | —CH₃/—CH₃ |
| IA-11/N-(6-{[5,5-Dimethyl-7-oxo-3-(1H-pyrazol-3-yl)-4,5,6,7 [symbol] | —(CH₂)₆—NH—(C═O)—CH₃ | —CH₃/—CH₃ |
| tetrahydro-2-benzothiophen-1-yl]sulfanyl}hexyl)acetamide | | |
| IA-12/3-[(3-Hydroxypropyl)sulfanyl]-6,6-dimethyl-1-(1H- | —(CH₂)₃—OH | —CH₃/—CH₃ |
| pyrazol-3-yl)-6,7-dihydro-2-benzothiophen-4(5H)-one | | |
| IA-13/3-[(3-Azidopropyl)sulfanyl]-6,6-dimethyl-1-(1H-pyrazo [symbol] | —(CH₂)₃—N═N⁺═N⁻ | —CH₃/—CH₃ |
| 3-yl)-6,7-dihydro-2-benzothiophen-4(5H)-one | | |
| IA-14/6,6-Dimethyl-3-[(2-methylbutyl)sulfanyl]-1-(1H-pyrazo [symbol] 3-yl)-6,7-dihydro-2-benzothiophen-4(5H)-one | 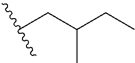 [SMILES: *CC(C)CC] | —CH₃/—CH₃ |
| IA-15/3-[(2-Hydroxypropyl)sulfanyl]-6,6-dimethyl-1-(1H- pyrazol-3-yl)-6,7-dihydro-2-benzothiophen-4(5H)-one | 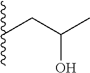 [SMILES: *CC(C)O] | —CH₃/—CH₃ |
| IA-16/6,6-Dimethyl-3-(methylsulfanyl)-1-(1H-pyrazol-3-yl)-6, [symbol] | —CH₃ | —CH₃/—CH₃ |
| dihydro-2-benzothiophen-4(5H)-one | | |
| IA-17/3-[(2-Hydroxyethyl)sulfanyl]-6,6-dimethyl-1-(1H-pyraz [symbol] | —(CH₂)₂—OH | —CH₃/—CH₃ |
| 3-yl)-6,7-dihydro-2-benzothiophen-4(5H)-one | | |
| IA-18/6,6-Dimethyl-1-(1H-pyrazol-3-yl)-3-[(2,2,2- | —CH₂—CF₃ | —CH₃/—CH₃ |
| trifluoroethyl)sulfanyl]-6,7-dihydro-2-benzothiophen-4(5H)-one | | |
| IA-19/3-(Ethylsulfanyl)-6,6-dimethyl-1-(1H-pyrazol-3-yl)-6,7- | —CH₂—CH₃ | —CH₃/—CH₃ |
| dihydro-2-benzothiophen-4(5H)-one | | |
| IA-20/3-[(2-Methoxyethyl)sulfanyl]-6,6-dimethyl-1-(1H-pyraz [symbol] | —(CH₂)₂—OCH₃ | —CH₃/—CH₃ |
| 3-yl)-6,7-dihydro-2-benzothiophen-4(5H)-one | | |
| IA-21/3-[(Furan-2-ylmethyl)sulfanyl]-6,6-dimethyl-1-(1H- pyrazol-3-yl)-6,7-dihydro-2-benzothiophen-4(5H)-one | 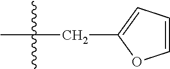 [SMILES: *Cc1ccco1] | —CH₃/—CH₃ |
| IA-22/3-[(6-Azidohexyl)sulfanyl]-6,6-dimethyl-1-(1H-pyrazol- | —(CH₂)₆—N═N⁺═N⁻ | —CH₃/—CH₃ |
| yl)-6,7-dihydro-2-benzothiophen-4(5H)-one | | |
| IA-23/3-{[2-(Dimethylamino)ethyl]sulfanyl}-6,6-dimethyl-1- | —(CH₂)₂—N—(CH₃)₂ | —CH₃/—CH₃ |
| (1H-pyrazol-3-yl)-6,7-dihydro-2-benzothiophen-4(5H)-one | | |
| IA-24/6,6-Dimethyl-3-[(2-phenylethyl)sulfanyl]-1-(1H-pyrazol [symbol] | —(CH₂)₂-phenyl | —CH₃/—CH₃ |
| 3-yl)-6,7-dihydro-2-benzothiophen-4(5H)-one | | |
| IA-25/3-[(2,3-Dihydroxypropyl)sulfanyl]-6,6-dimethyl-1-(1H- pyrazol-3-yl)-6,7-dihydro-2-benzothiophen-4(5H)-one | 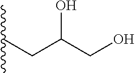 [SMILES: *CC(O)CO] | —CH₃/—CH₃ |
| IA-26/3-(Benzylsulfanyl)-6,6-dimethyl-1-(1H-pyrazol-3-yl)-6, [symbol] | —CH₂-phenyl | —CH₃/—CH₃ |
| dihydro-2-benzothiophen-4(5H)-one | | |
| IA-27/3-(Hexylsulfanyl)-6,6-dimethyl-1-(1H-pyrazol-3-yl)-6,7 [symbol] | —(CH₂)₅—CH₃ | —CH₃/—CH₃ |
| dihydro-2-benzothiophon-4(5H)-one | | |
| IA-28/6,6-Dimethyl-1-(1H-pyrazol-3-yl)-3-{[2- | —(CH₂)₂—Si(CH₃)₃ | —CH₃/—CH₃ |
| (trimethylsilyl)ethyl]sulfanyl}-6,7-dihydro-2-benzothiophen- | | |
| 4(5H)-one | | |
| IA-29/6,6-Dimethyl-3-[(5-methyl-1,3,4-thiadiazol-2-yl)sulfany [symbol] 1-(1H-pyrazol-3-yl)-6,7-dihydro-2-benzothiophen-4(5H)-one | 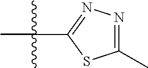 [SMILES: *c1nnc(C)s1] | —CH₃/—CH₃ |
| IA-30/3-(Methylsulfanyl)-1-(1H-pyrazol-3-yl)-6,7-dihydro-2- | —CH₃ | —H/—H |
| benzothiophen-4(5H)-one | | |
| IA-31/3-[(6-Aminohexyl)sulfanyl]-6,6-dimethyl-1-(1H-pyrazo [symbol] | —(CH₂)₆—NH₂ | —CH₃/—CH₃ |
| 3-yl)-6,7-dihydro-2-benzothiophen-4(5H)-one. | | |

[symbol]

indicates data missing or illegible when filed

In some embodiments it is preferred to incorporate into a formulation of the invention at least one of the compounds of Formula IB:

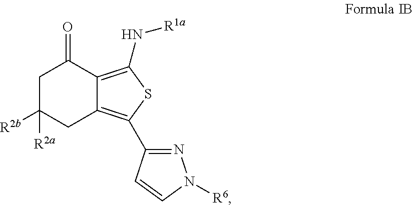

wherein —R^{1a}, —R^{2a}, and —R^{2b} are defined below in Table IV.

TABLE IV

| Cmpd. No./Nomenclature | —R^{1a} | R^{2a}/R^{2b} | R⁶ |
|---|---|---|---|
| IB-01/3-(Cyclopentylamino)-1-(1H-pyrazol-3 [symbol] yl)-6,7-dihydro-2-benzothiophen-4(5H)-one | 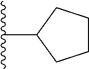 [SMILES: *C1CCCC1] | —H/—H | —H |
| IB-02/3-(Cyclopentylamino)-6,6-dimethyl-1- | ″ | —CH₃/—CH₃ | —H |
| (1H-pyrazol-3-yl)-6,7-dihydro-2-benzothiophe [symbol] | | | |
| 4(5H)-one | | | |
| IB-03/N-(4-cyanophenyl)-3-[3- (cyclopentylamino)-4-oxo-4,5,6,7-tetrahydro-2 [symbol] benzothiophen-1-yl]-1H-pyrazole-1- carboxamide | ″ | —H/—H | 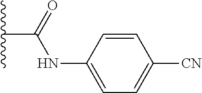 [SMILES: *C(=O)Nc1ccc(C#N)cc1] |
| IB-04/N-(4-Cyanophenyl)-3-[3- (cyclopentylamino)-6,6-dimethyl-4-oxo-4,5,6,7 [symbol] tetrahydro-2-benzothiophen-1-yl]-1H-pyrazole- 1-carboxamide | ″ | —CH₃/—CH₃ | 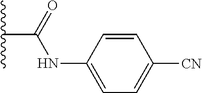 [SMILES: *C(=O)Nc1ccc(C#N)cc1] |
| IB-05/N-(4-Chlorophenyl)-3-[3- (cyclopentylamino)-6,6-dimethyl-4-oxo-4,5,6,7 [symbol] tetrahydro-2-benzothiophen-1-yl]-1H-pyrazole- 1-carboxamide | ″ | —CH₃/—CH₃ | 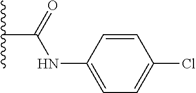 [SMILES: *C(=O)Nc1ccc(Cl)cc1] |
| IB-06/3-[3-(Cyclopentylamino)-6,6-dimethyl- 4-oxo-4,5,6,7-tetrahydro-2-benzothiophen-1-yl [symbol] N-(pyridin-4-yl)-1H-pyrazole-1-carboxamide | ″ | —CH₃/—CH₃ | 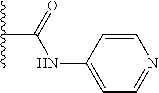 [SMILES: *C(=O)Nc1ccncc1] |
| IB-07/6,6-Dimethyl-3-(methylamino)-1-(1H- | —CH₃ | —CH₃/—CH₃ | —H |
| pyrazol-3-yl)-6,7-dihydro-2-benzothiophen- | | | |
| 4(5H)-one | | | |
| IB-08/3-(Ethylamino)-6,6-dimethyl-1-(1H- | —CH₂—CH₃ | —CH₃/—CH₃ | —H |
| pyrazol-3-yl)-6,7-dihydro-2-benzothiophen- | | | |
| 4(5H)-one | | | |
| IB-09/6,6-Dimethyl-3-(propylamino)-1-(1H- | —(CH₂)₂—CH₃ | —CH₃/—CH₃ | —H |
| pyrazol-3-yl)-6,7-dihydro-2-benzothiophen- | | | |
| 4(5H)-one | | | |
| IB-10/6,6-Dimethyl-3-(propan-2-ylamino)-1- | —CH(CH₃)₂ | —CH₃/—CH₃ | —H |
| (1H-pyrazol-3-yl)-6,7-dihydro-2-benzothiophe [symbol] | | | |
| 4(5H)-one | | | |
| IB-11/3-[(6-Hydroxyhexyl)amino]-6,6- | —(CH₂)₆—OH | —CH₃/—CH₃ | —H |
| dimethyl-1-(1H-pyrazol-3-yl)-6,7-dihydro-2- | | | |
| benzothiophen-4(5H)-one | | | |
| IB-12/3-(Cyclopropylamino)-6,6-dimethyl-1- (1H-pyrazol-3-yl)-6,7-dihydro-2-benzothiophe [symbol] 4(5H)-one | 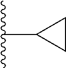 [SMILES: *C1CC1] | —CH₃/—CH₃ | —H |
| IB-13/3-[(trans-4-Hydroxycyclohexyl)amino] [symbol] 6,6-dimethyl-1-(1H-pyrazol-3-yl)-6,7-dihydro- 2-benzothiophen-4(5H)-one | 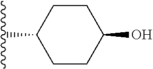 [SMILES: *[C@H]1CC[C@H](O)CC1] | —CH₃/—CH₃ | —H |
| IB-14/3-(Cyclohexylamino)-6,6-dimethyl-1- (1H-pyrazol-3-yl)-6,7-dihydro-2-benzothiophe [symbol] 4(5H)-one | 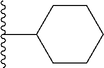 [SMILES: *C1CCCCC1] | —CH₃/—CH₃ | —H |
| IB-15/6,6-Dimethyl-3-{[2-(morpholin-4- yl)ethyl]amino}-1-(1H-pyrazol-3-yl)-6,7- dihydro-2-benzothiophen-4(5H)-one | 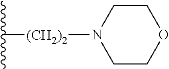 | —CH₃/—CH₃ | —H |
| IB-16/6,6-Dimethyl-1-(1H-pyrazol-3-yl)-3- [(pyridin-2-ylmethyl)amino]-6,7-dihydro-2- benzothiophen-4(5H)-one | 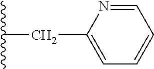 [SMILES: *Cc1ccccn1] | —CH₃/—CH₃ | —H |
| IB-17/6,6-Dimethyl-1-(1H-pyrazol-3-yl)-3- {[2-(pyridin-2-yl)ethyl]amino)-6,7-dihydro-2- benzothiophen-4(5H)-one | 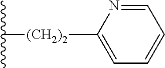 | —CH₃/—CH₃ | —H |
| IB-18/3-[(Furan-2-ylmethyl)amino]-6,6- dimethyl-1-(1H-pyrazol-3-yl)-6,7-dihydro-2- benzothiophen-4(5H)-one | 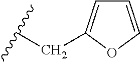 [SMILES: *Cc1ccco1] | —CH₃/—CH₃ | —H |
| IB-19/6,6-Dimethyl-3-{[3-(4-methylpiperazi [symbol] 1-yl)propyl]amino}-1-(1H-pyrazol-3-yl)-6,7- dihydro-2-benzothiophen-4(5H)-one | 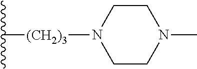 | —CH₃/—CH₃ | —H |

[symbol]

indicates data missing or illegible when filed

or one of the following compounds:

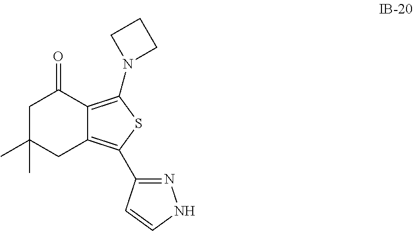 [SMILES: CC1(C)CC(=O)c2c(N3CCC3)sc(-c3cc[nH]n3)c2C1]

(3-(Azetidin-1-yl)-6,6-dimethyl-1-(1H-pyrazol-3-yl)-6,7-dihydro-2-benzothiophen-4(5H)-one);

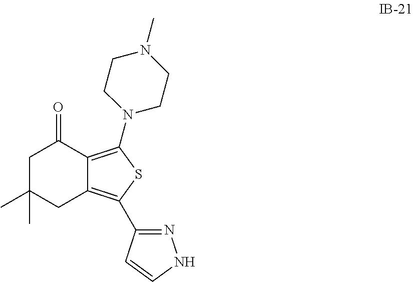 [SMILES: CN1CCN(c2sc(-c3cc[nH]n3)c3c2C(=O)CC(C)(C)C3)CC1]

(6,6-Dimethyl-3-(4-methylpiperazin-1-yl)-1-(1H-pyrazol-3-yl)-6,7-dihydro-2-benzothiophen-4(5H)-one); or

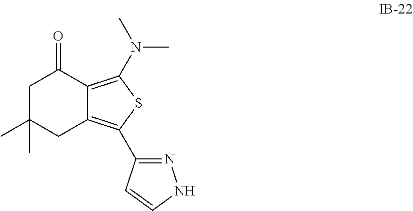 [SMILES: CN(C)c1sc(-c2cc[nH]n2)c2c1C(=O)CC(C)(C)C2]

(3-(Dimethylamino)-6,6-dimethyl-1-(1H-pyrazol-3-yl)-6,7-dihydro-2-benzothiophen-4(5H-one).

In some embodiments it is preferred to provide a formulation comprising at least one compound of Formula IC:

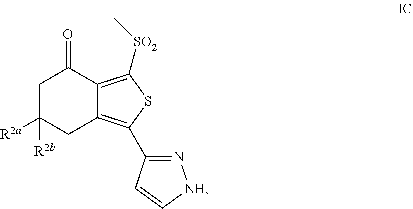

wherein both of —R^{2a} and —R^{2b} are methyl (Formula IC-01, 6,6-Dimethyl-3-(methylsulfonyl)-1-(1H-pyrazol-3-yl)-6,7-dihydro-2-benzothiophen-4(5H)-one), or both of —R^{2a} and —R^{2b} are —H (Formula IC-02, 3-(Methylsulfonyl)-1-(1H-pyrazol-3-yl)-6,7-dihydro-2-benzothiophen-4(5H)-one).

In some embodiments it is preferred to provide a formulation of the invention comprising at least one compound of Formula ID:

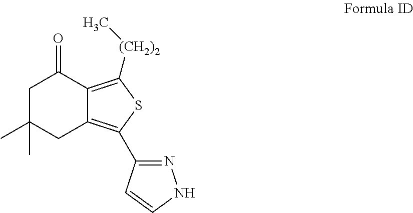

(6,6-Dimethyl-3-propyl-1-(1H-pyrazol-3-yl)-6,7-dihydro-2-benzothiophen-4(5H)-one).

In some embodiments it is preferred to provide a formulation of the invention comprising at least one compound of Formula IE:

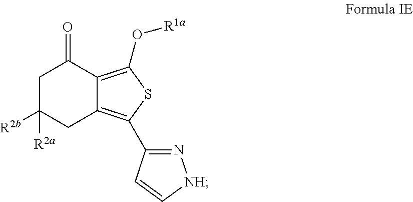

wherein —R^{1a}, —R^{2a}, and —R^{2b} are defined below in Table V.

TABLE V

| Cmpd. No./Nomenclature | —R^{1a} | —R^{2a}/—R^{2b} |
|---|---|---|
| IE-01/3-(Cyclopentyloxy)-6,6-dimethyl-1-(1H-pyrazol-3-yl)-6, [symbol] dihydro-2-benzothiophen-4(5H)-one | 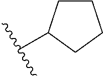 [SMILES: *C1CCCC1] | —CH₃/—CH₃ |
| IE-02/3-(Cyclobutyloxy)-6,6-dimethyl-1-(1H-pyrazol-3-yl)-6,7 [symbol] dihydro-2-benzothiophen-4(5H)-one | 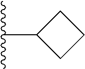 [SMILES: *C1CCC1] | —CH₃/—CH₃ |
| IE-03/6,6-Dimethyl-3-phenoxy-1-(1H-pyrazol-3-yl)-6,7-dihyd [symbol] 2-benzothiophen-4(5H)-one | 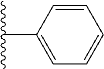 [SMILES: *c1ccccc1] | —CH₃/—CH₃ |
| IE-04/6,6-Dimethyl-3-(propan-2-yloxy)-1-(1H-pyrazol-3-yl)-6 [symbol] dihydro-2-benzothiophen-4(5H)-one | 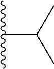 [SMILES: *C(C)C] | —CH₃/—CH₃ |

[symbol]

indicates data missing or illegible when filed

In some embodiments it is preferred to provide a formulation of the invention comprising a compound of Formula IF:

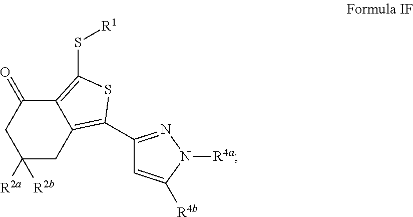

wherein substituents —R¹, —R^{2a}, —R^{2b}, —R^{4a} and —R^{4b} are defined in Tables V, VI, below.

In some embodiments it is preferred to provide a formulation of the invention comprising a compound of Formula IF wherein —R¹ is “-cyclopentyl”, —R^{4b} is “—H” and —R^{4a}, —R^{2a} and —R^{2b} are as defined in Table VI.

TABLE VI

| Cmpd. No./Nomenclature | —R^{2a}/—R^{2b} | —R^{4a} |
|---|---|---|
| IF-01/3-[3-(Cyclopentylsulfanyl)-6,6- | —CH₃/—CH₃ | —C═O—NH—CH₃ |
| dimethyl-4-oxo-4,5,6,7-tetrahydro-2- | | |
| benzothiophen-1-yl]-N-methyl-1H-pyrazole- | | |
| 1-carboxamide | | |
| IF-02/3-[3-(Cyclopentylsulfanyl)-6,6- | —CH₃/—CH₃ | —C═O—NH—CH(CH₃)₂ |
| dimethyl-4-oxo-4,5,6,7-tetrahydro-2- | | |
| benzothiophen-1-yl)-N-(propan-2-yl)-1H- | | |
| pyrazole-1-carboxamide | | |
| IF-03/N-tert-Butyl-3-[3-(cyclopentyl- | —CH₃/—CH₃ | —C═O—NH—C(CH₃)₃ |
| sulfanyl)-6,6-dimethyl-4-oxo-4,5,6,7- | | |
| tetrahydro-2-benzothiophen-1-yl]-1H- | | |
| pyrazole-1-carboxamide | | |
| IF-04/N-(Butan-2-yl)-3-[3-(cyclopentyl- sulfanyl)-6,6-dimethyl-4-oxo-4,5,6,7- tetrahydro-2-benzothiophen-1-yl]-1H- pyrazole-1-carboxamide | —CH₃/—CH₃ | 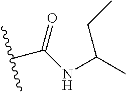 [SMILES: *C(=O)NC(C)CC] |
| IF-05/3-[3-(Cyclopentylsulfanyl)-6,6- dimethyl-4-oxo-4,5,6,7-tetrahydro-2- benzothiophen-1-yl]-N-[1- (trifluoroacetyl)piperidin-4-yl]-1H-pyrazole- 1-carboxamide | —CH₃/—CH₃ | 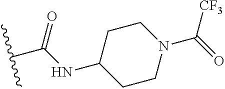 [SMILES: *C(=O)NC1CCN(C(=O)C(F)(F)F)CC1] |
| IF-06/3-[3-(Cyclopentylsulfanyl)-6,6- ditnethyl-4-oxo-4,5,6,7-tetrahydro-2- benzothiophen-1-yl]-N-(4-methoxyphenyl)- 1H-pyrazole-1-carboxamide | —CH₃/—CH₃ | 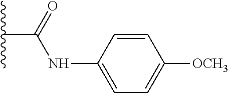 [SMILES: *C(=O)Nc1ccc(OC)cc1] |
| IF-07/3-[3-(Cyclopentylsulfanyl)-6,6- dimethyl-4-oxo-4,5,6,7-tetrahydro-2- benzothiophen-1-yl]-N-(2-methoxyphenyl)- 1H-pyrazole-1-carboxamide | —CH₃/—CH₃ | 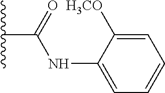 [SMILES: *C(=O)Nc1ccccc1OC] |
| IF-08/3-[3-(Cyclopentylsulfanyl)-6,6- dimethyl-4-oxo-4,5,6,7-tetrahydro-2- benzothiophen-1-yl]-N-[3- (methylsulfanyl)phenyl]-1H-pyrazole-1- carboxamide | —CH₃/—CH₃ | 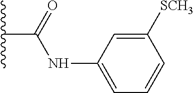 [SMILES: *C(=O)Nc1cccc(SC)c1] |
| IF-09/3-[3-(Cyclopentylsulfanyl)-6,6- dimethyl-4-oxo-4,5,6,7-tetrahydro-2- benzothiophen-1-yl]-N-(naphthalen-2-yl)- 1H-pyrazole-1-carboxamide | —CH₃/—CH₃ | 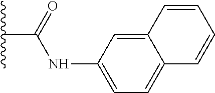 [SMILES: *C(=O)Nc1ccc2ccccc2c1] |
| IF-10/3-[3-(Cyclopentylsulfanyl)-6,6- dimethyl-4-oxo-4,5,6,7-tetrahydro-2- benzothiophen-1-yl]-N-[4- (trifluoromethyl)phenyl]-1H-pyrazole-1- carboxamide | —CH₃/—CH₃ | 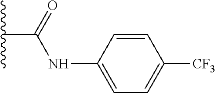 [SMILES: *C(=O)Nc1ccc(C(F)(F)F)cc1] |
| IF-11/N-benzyl-3-[3-(Cyclopentylsulfanyl)- 6,6-dimethyl-4-oxo-4,5,6,7-tetrahydro-2- benzothiophen-1-yl]-1H-pyrazole-1- carboxamide | —CH₃/—CH₃ | 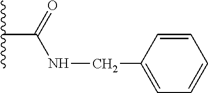 [SMILES: *C(=O)NCc1ccccc1] |
| IF-12/3-[3-(Cyclopentylsulfanyl)-6,6- dimethyl-4-oxo-4,5,6,7-tetrahydro-2- benzothiophen-1-yl]-N-(2-phenylethyl)-1H- pyrazole-1-carboxamide | —CH₃/—CH₃ | 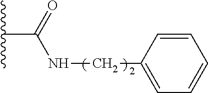 |
| IF-13/N-(4-Chlorophenyl)-3-[3- (cyclopentylsulfanyl)-6,6-dimethyl-4-oxo- 4,5,6,7-tetrahydro-2-benzothiophen-1-yl]- 1H-pyrazole-1-carboxamide | —CH₃/—CH₃ | 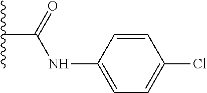 [SMILES: *C(=O)Nc1ccc(Cl)cc1] |
| IF-14/N-(4-chlorophenyl)-3-[3- (cyclopentylsulfanyl)-4-oxo-4,5,6,7- tetrahydro-2-benzothiophen-1-yl]-1H- pyrazole-1-carboxamide | —H/—H | 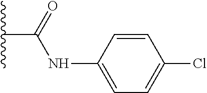 [SMILES: *C(=O)Nc1ccc(Cl)cc1] |
| IF-15/N-(4-Cyanophenyl)-3-[3- (cyclopentylsulfanyl)-6,6-dimethyl-4-oxo- 4,5,6,7-tetrahydro-2-benzothiophen-1-yl]- 1H-pyrazole-1-carboxamide | —CH₃/—CH₃ | 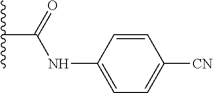 [SMILES: *C(=O)Nc1ccc(C#N)cc1] |
| IF-16/N-(3-Cyanophenyl)-3-[3- (cyclopentylsulfanyl)-6,6-dimethyl-4-oxo- 4,5,6,7-tetrahydro-2-benzothiophen-1-yl]- 1H-pyrazole-1-carboxamide | —CH₃/—CH₃ | 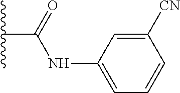 [SMILES: *C(=O)Nc1cccc(C#N)c1] |
| IF-17/N-(3-Chlorophenyl)-3-[3- (cyclopentylsulfanyl)-6,6-dimethyl-4-oxo- 4,5,6,7-tetrahydro-2-benzothiophen-1-yl]- 1H-pyrazole-1-carboxamide | —CH₃/—CH₃ | 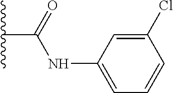 [SMILES: *C(=O)Nc1cccc(Cl)c1] |
| IF-18/1-(1-Butanoyl-1H-pyrazol-3-yl)-3- | —CH₃/—CH₃ | —(C═O)—(CH₂)₂—CH₃ |
| (cyclopentylsulfanyl)-6,6-dimethyl-6,7- | | |
| dihydro-2-benzothiophen-4(5H)-one | | |
| IF-19/3-(Cyclopentylsulfanyl)-6,6- | —CH₃/—CH₃ | —(C═O)—CH(CH₃)₂ |
| dimethyl-1-[1-(2-methylpropanoyl)-1H- | | |
| pyrazol-3-yl]-6,7-dihydro-2-benzothiophen- | | |
| 4(5H)-one | | |
| IF-20/4-({3-[3-(Cyclopentylsulfanyl)-6,6- dimethyl-4-oxo-4,5,6,7-tetrahydro-2- benzothiophen-1-yl]-1H-pyrazol-1- yl}carbonyl)benzonitrile | —CH₃/—CH₃ | 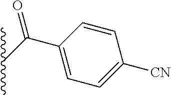 [SMILES: *C(=O)c1ccc(C#N)cc1] |
| IF-21/3-(Cyclopentylsulfanyl)-1-{1-[(4- methoxyphenyl)carbonyl]-1H-pyrazol-3-yl}- 6,6-dimethyl-6,7-dihydro-2-benzothiophen- 4(5H)-one | —CH₃/—CH₃ | 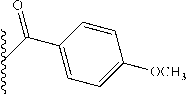 [SMILES: *C(=O)c1ccc(OC)cc1] |
| IF-22/3-(Cyclopentylsulfanyl)-1-{1-[(3,4- difluorophenyl)carbohyl]-1H-pyrazol-3-yl}- 6,6-dimethyl-6,7-dihydro-2-benzothiophen- 4(5H)-one | —CH₃/—CH₃ | 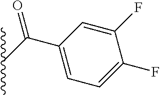 [SMILES: *C(=O)c1ccc(F)c(F)c1] |
| IF-23/3-(Cyclopentylsulfanyl)-1-{1-[(4- fluorophenyl)carbonyl)-1H-pyrazol-3-yl}- 6,6-dimethyl-6,7-dihydro-2-benzothiophen- 4(5H)-one | —CH₃/—CH₃ | 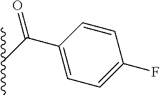 [SMILES: *C(=O)c1ccc(F)cc1] |
| IF-24/1-{1-[(4-Chlorophenyl)acetyl]-1H- pyrazol-3-yl}-3-(cyclopentylsulfanyl)-6,6- dimethyl-6,7-dihydro-2-benzothiophen- 4(5H)-one | —CH₃/—CH₃ | 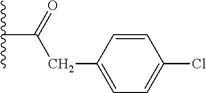 [SMILES: *C(=O)Cc1ccc(Cl)cc1] |
| IF-25/1-{1-[(6-Chloropyridin-3- yl)carbonyl]-1H-pyrazol-3-yl}-3- (cyclopentylsulfanyl)-6,6-dimethyl-6,7- dihydro-2-benzothiophen-4(5H)-one | —CH₃/—CH₃ | 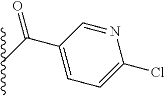 [SMILES: *C(=O)c1ccc(Cl)nc1] |
| IF-26/1-[1-(Cyclohexylcarbonyl)-1H- pyrazol-3-yl]-3-(cyclopentylsulfanyl)-6,6- dimethyl-6,7-dihydro-2-benzothiophen- 4(5H)-one | —CH₃/—CH₃ | 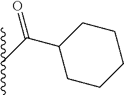 [SMILES: *C(=O)C1CCCCC1] |

In some embodiments it is preferred to provide a formulation of the invention comprising a compound of Formula IF wherein —R¹ is —CH₃, R^{4a} is —H and R^{4b}, —R^{2a} and —R^{2b} are as defined in Table VII.

TABLE VII

| Cmpd. No./Nomenclature | —R^{2a}/—R^{2b} | R^{4b} |
|---|---|---|
| IF-27/6,6-Dimethyl-1-(5-methyl-1H- | —CH₃/—CH₃ | —CH₃ |
| pyrazol-3-yl)-3-(methylsulfanyl)-6,7- | | |
| dihydro-2-benzothiophen-4(5H)-one | | |
| IF-28/1-(5-Cyclopropyl-1H-pyrazol- 3-yl)-6,6-dimethyl-3-(methylsulfanyl)- 6,7-dihydro-2-benzothiophen-4(5H)-one | —CH₃/—CH₃ | 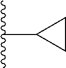 [SMILES: *C1CC1] |
| IF-29/1-(5-Ethyl-1H-pyrazol-3-yl)-6,6- | —CH₃/—CH₃ | —CH₂—CH₃ |
| dimethyl-3-(methylsulfanyl)-6,7- | | |
| dihydro-2-benzothiophen-4(5H)-one | | |

In some embodiments it is preferred to provide a formulation of the invention comprising a compound of Formula IF-30:

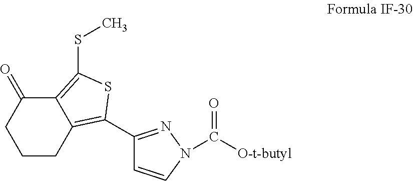

(tert-Butyl 3-[3-(methylsulfanyl)-4-oxo-4,5,6,7-tetrahydro-2-benzothiophen-1-yl]-1H-pyrazole-1-carboxylate).

In some embodiments it is preferred to provide a formulation of the invention comprising a compound of Formula IG:

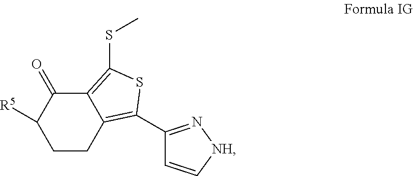

wherein, —R⁵ is -methyl, thus the compound of Formula IG-01 [5-Methyl-3-(methylsulfanyl)-1-(1H-pyrazol-3-yl)-6,7-dihydro-2-benzothiophen-4(5H)-one], or n-propyl, thus the compound of Formula IG-02 [3-(Methylsulfanyl)-5-propyl-1-(1H-pyrazol-3-yl)-6,7-dihydro-2-benzothiophen-4(5/1)-one].

In some embodiments the invention provides also novel compounds of Formula I, for example, with reference to Table III above, the compounds of Formulae: IA-01 to IA-03; IA-05; IA-07; IA-10; IA-011; IA-13; IA-14; IA-21 to IA-23; IA-28 to IA-31, with reference to Table IV, above, the compounds of Formulae: IB-01 to IB-06; and IB-11 to IB-19, with reference to Table V, above, the compounds of Formulae: IE-01 to IE-03; with reference to Tables VI and VII, above, the compounds of Formulae: IF-01 to IF-29, and the compounds of Formulae IB-20, IB-21, IB-22, IC-02, ID, IF-30, IG-01, and IG-02.

In some embodiments, the compounds of Formulae IA-01, IA-02, IA-31, IB-02, IF-10, IF-13, IF-14, and IF-15 are preferred novel compounds. In some embodiments, the compounds of Formulae IA-03 to IA-19, IA-30, IB-01, IB-02, IB08 to IB-14, IF-06 to IF09, IF-16, IG-01, and IG-02 are preferred novel compounds. In some embodiments, compounds of the following Formula are preferred novel compounds: IA-20 to IA-22, IB-03, IB-05, IB-15, IB-17 to IB-19, IE-01, IE-02, IF-05, IF-17, IF-20, IF-21, IF-28, and IF-29.

It will be appreciated that the foregoing novel compounds are claimed as well in pure form or in isolated form as defined herein. It will be appreciated also that these compounds can comprise multiple stereocenters, accordingly, stereoisomers and diastereomers in all possible combinations and racemates are included in the description of the compounds of the aforementioned Formulae. It will be appreciated as well that all isolated forms, for example, isolated pure stereoisomers, mixtures of diastereomers, and racemates as well as conventionally obtained amorphous, and crystalline forms and solvates, hydrates, and tautomers of the compounds available by known methods are included in the description of the novel compounds of Formula I described above, as well as salt forms available by known means derived from the novel compounds of Formula I described above.

Another embodiment of the invention is administration of a formulation of the invention which is a pharmaceutical composition comprising an effective amount of at least one compound of Formula I (e.g., 1, 2 or 3, or 1 or 2, or 1, and usually 1), or a pharmaceutically acceptable salt thereof, and at least one compound of Formula I or a salt thereof, in any isolated form, and at least one pharmaceutically acceptable excipient. Methods for the safe and effective administration of compounds of Formula I are known to those skilled in the art, for example, as described in the standard literature, for example, as described in the “Physicians' Desk Reference” (PDR), e.g., 1996 edition (Medical Economics Company, Montvale, N.J. 07645-1742, USA), the Physician's Desk Reference, 56^{th} Edition, 2002 (published by Medical Economics company, Inc. Montvale, N.J. 07645-1742), or the Physician's Desk Reference, 57^{th} Edition, 2003 (published by Thompson PDR, Montvale, N.J. 07645-1742); the disclosures of which is incorporated herein by reference thereto.

Examples of methods of administering a compound of Formula I include incorporating it into a pharmaceutical composition adapted for administration orally, via mucosal absorption, or for injection or intravenous delivery. Examples of such delivery methods include, for example, but are not limited to, a pharmaceutical composition comprising at least one compound of Formula I adapted for: (i) oral administration, e.g., a liquid, gel, powder, solid or semi-solid pharmaceutical composition which is loaded into a capsule or pressed into a tablet; (ii) a solution or suspension adapted for intramuscular administration (IM); (iii) a solution or suspension adapted for intravenous administration (IV), for example, as an IV solution or a concentrate to be injected into a saline IV bag; (iv) a lozenge form for administration through tissues of the oral cavity; (v) a solution, suspension or emulsion formulation for dispersion administration via the nasal mucosa; (vi) a suppository form for administration via the rectal or vaginal mucosa. The use of a pharmaceutical composition comprising more than one compound of Formula I, and comprising more than one type of pharmaceutically active compound is within the scope of this invention.

For preparing pharmaceutical compositions from the compounds described by this invention, generally pharmaceutically active compounds are combined with one or more pharmaceutically inactive excipients. These pharmaceutically inactive excipients impart to the composition properties which make it easier to handle or process, for example, lubricants or pressing aids in powdered medicaments intended to be tableted, or adapt the formulation to a desired route of administration, for example, excipients which provide a formulation for oral administration, for example, via absorption from the gastrointestinal tract, transdermal or transmucosal administration, for example, via adhesive skin “patch” or buccal administration, or injection, for example, intramuscular or intravenous, routes of administration. These excipients are collectively termed herein “a carrier”.

Pharmaceutical compositions can be solid, semi-solid or liquid. Solid form preparations can be adapted to a variety of modes of administration and include powders, dispersible granules, mini-tablets, beads, and the like for example, for tableting, encapsulation, or direct administration. Typically formulations may comprise up to about 95 percent active ingredient, although formulations with greater amounts may be prepared.

Liquid form preparations include solutions, suspensions and emulsions. Examples of liquid forms of medicament include, but are not limited to, water or water/surfactant mixtures, for example a water-propylene glycol solution, which can be employed in the preparation of formulations intended, for example, for parenteral injection, for example, as a solvent or as a suspending medium for the preparation of suspensions and emulsions where a medicament comprises constituents which are insoluble in water or water/surfactant mixtures. Liquid form preparations may also include solutions for intranasal administration which may also include, for example, viscosity modifiers to adapt the formulation to target application of the formulation to particular mucosa tissues accessible via nasal administration.

Aerosol preparations, for example, suitable for administration via inhalation or via nasal mucosa, may include solutions and solids in powder form, which may be in combination with a pharmaceutically acceptable propellant, for example, an inert compressed gas, e.g. nitrogen. Also included are solid form preparations which are intended to be converted, shortly before use, to a suspension, solution, or a solution, for example, for oral or parenteral administration. Examples of such solid forms include freeze dried formulations and liquid formulations adsorbed into a solid absorbent medium.

The compounds of the invention may also be deliverable transdermally or transmucosally, for example, from a liquid, suppository, cream, foam, gel, or rapidly dissolving solid form. It will be appreciated that transdermal compositions can take also the form of creams, lotions, aerosols and/or emulsions and can be provided in a unit dosage form which includes a transdermal patch of any know in the art, for example, a patch which incorporates either a matrix comprising the pharmaceutically active compound or a reservoir which comprises a solid or liquid form of the pharmaceutically active compound.

Examples of pharmaceutically acceptable carriers and methods of manufacture for various compositions mentioned above may be found in A. Gennaro (ed.), Remington: The Science and Practice of Pharmacy, 20^{th} Edition, (2000), Lippincott Williams & Wilkins, Baltimore, Md.

Preferably, the pharmaceutical preparation is in a unit dosage form. In such form, the preparations subdivided into suitably sized unit doses containing appropriate quantities of the active component, e.g., an effective amount to achieve the desired purpose.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in two to four divided doses.

In general, in what ever form administered, the amount of a pharmaceutical composition comprising at least one compound of the invention, for example, a compound of Formula I, that will be administered will be that amount providing a therapeutic serum level of the compound for a period of at least 2 hours, preferably at least four hours, and preferably longer. In general, as is known in the art, dosages of a pharmaceutical composition providing a therapeutically effective serum level of a compound of the invention, e.g., a compound of Formula I, are spaced in time to provide serum level meeting or exceeding the minimum therapeutically effective serum level on a continuous basis throughout the period during which treatment is administered.

As mentioned in the definitions above, administration of multiple pharmaceutically active compounds in connection with LRRK2 inhibiting compounds of the invention, or the administration of more than one compound of the invention in the provision of a treatment or management of a disease state, can comprise, administering a single pharmaceutical composition comprising all of the pharmaceutically active compounds or multiple compositions comprising one or more pharmaceutically active compounds. As mentioned in the “definitions” section, above, administration of more that one pharmaceutical composition can comprise simultaneous, contemporaneous, or sequential administration of said pharmaceutical compositions.

Pharmaceutical compositions of the invention may also include other compounds having pharmaceutical activity, that is, activity which treats, manages, mitigates, ameliorates, improves, eliminates, or cures a disease state or symptom associated with a disease state, for example, a composition comprising an effective amount of one or more compounds of Formula I and in addition an effective amount of another therapeutic agent, for example: L-DOPA; dopaminergic agonists such as quinpirole, ropinirole, pramipexole, pergolide and bromocriptine; MAO-B inhibitors such as rasagiline, deprenyl and selegiline; DOPA decarboxylase inhibitors such as carbidopa and benserazide; and COMT inhibitors such as tolcapone and entacapone; or potential therapies such as an adenosine Ata antagonists, metabotropic glutamate receptor 4 modulators, or growth factors such as brain derived neurotrophic factor (BDNF), and a pharmaceutically acceptable carrier. and optionally one or more pharmaceutically acceptable excipients.

Another embodiment of this invention is directed to a method of treating or managing at least one symptom associated with Parkinson's disease in a patient in need of such treatment, said method comprising administering to said patient an effective amount of at least one compound of Formula I.

It will be appreciated that any of the methods of treating Parkinson's disease described herein, unless stated otherwise, can optionally include the administration of an effective amount of one or more (e.g., 1, 2 or 3, or 1 or 2, or 1) agents effective in treating movement disorders associated with Parkinson's disease or side-effects arising from administering agents effective in treating Parkinson's disease. It will be appreciated also that when more than one pharmaceutically active compound is administered, pharmaceutically active compounds, including the administration of multiple compounds of this invention, can be administered together in the same formulation, or can be administered concurrently, contemporaneously, or sequentially in separate formulations.

Examples of disorders or disease states which may be managed, ameliorated, alleviated or treated by the methods of this invention include, but are not limited to: Parkinson's disease, Alzheimer's disease, Huntington's disease, dystonia, essential tremor, cognitive impairment and dementia, depression, anxiety, impulse control disorders, restless legs syndrome, excessive daytime sleepiness, insomnia, gastric disturbances or other autonomic nervous system dysfunction, or non-skin cancers associated with mutant LRRK2 function.

The compounds of Formula I, including the compounds of Formulae IA, IB, IC, ID, IE, IF, IG, IH, and IJ described above, inhibit Leucine-Rich Repeat Kinase-2 activity, thus, this invention further provides a method of inhibiting kinase activity in mammals, especially humans, by the administration of an effective amount (e.g., a therapeutically effective amount) of one or more (e.g., one) compounds of Formula I. Thus, one embodiment of this invention is directed to a method of inhibiting LRRK2 activity (i.e., inhibiting the enzymatic activity Leucine-Rich Repeat Kinase protein) in a patient in need of such treatment comprising administering to said patient an effective amount of at least one compound of Formula I. Another embodiment of this invention is directed to a method of inhibiting LRRK2 activity in a patient in need of such treatment comprising administering to said patient an effective amount of at least one compound of Formula I, preferably a compound of Formula IA, IB, IC, ID, IE, IF, or IG, as described above and defined in Tables III to VII, above.

Those skilled in the art will appreciate that treatment protocols utilizing at least one compound of Formula I can be varied according to the needs of the patient. Thus, compounds of Formula I used in the methods of this invention can be administered in variations of the protocols described above. For example, the compounds of this invention can be administered discontinuously rather than continuously during the treatment cycle.

Other embodiments of this invention are directed to any one of the embodiments above of managing, ameliorating, alleviating or treating disease states which include, but are not limited to: Parkinson's disease, Alzheimer's disease, Huntington's disease, dystonia, essential tremor, cognitive impairment and dementia, depression, anxiety, impulse control disorders, restless legs syndrome, excessive daytime sleepiness, insomnia, gastric disturbances and other autonomic nervous system dysfunction, and cancers associated with mutant LRRK2 function, wherein the compound of Formula I administered is a compound of any of IA, IB, IC, ID, IE, IF, or IG as described above and defined above in Tables III to VII.

### EXAMPLES

The compounds of the invention can be made according to the general processes described below by selecting the appropriate reagents.

The following abbreviations have the following meanings unless defined otherwise: ACN=Acetonitrile; AcOH=Acetic acid; DAST=(diethylamino)sulfur trifluoride; DCC=Dicyclohexylcarbodiimide; DCU=Dicyclohexylurea; DCM=Dichloromethane; DI=Deionized water; DIAD=Diisopropylazodicarboxylate; DIEA=Diisopropylethylamine; DMAP=4-Dimethylaminopyridine; DME=Dimethoxyethane; DMF=Dimethylformamide; DMFDMA=N,N-Dimethylformamide dimethylacetal; DMSO=Dimethyl sulfoxide; DTT=Dithiothreitol; EDCI=1-(3-dimethylamino-propyl)-3-ethylcarbodiimide hydrochloride; EtOAc=Ethyl acetate; EtOH=Ethanol; HATU=N,N,N′,N′-Tetramethyl-O-(7-Azabenzotriazol-1-yl)Uronium hexafluorophosphate; Hex=hexanes; HOBt=1-Hydroxylbenzotriazole; HPLC=High pressure liquid chromatography; LCMS=Liquid chromatography mass spectrometry; LDA=Lithium diisopropylamide; mCPBA=meta-Chloroperoxybenzoic acid; MeOH=Methanol; MTT=(3-[4,5-dimethyl-thiazol-2-yl]-2,5-diphenyltetrazolium bromide, Thiazolyl blue); NMR=Nuclear magnetic resonance; PFP=Pentafluorophenol; PMB=p-methoxybenzyl; Pyr=Pyridine; Rb=Round bottom flask; Rbt=Round bottom flask; RT=Room temperature; SEMCl=2-(Trimethylsily)ethoxy methyl chloride; TEA=Triethylamine; Tr=Triphenyl methane; Trt=Triphenyl methane; TrCl=Triphenyl methane chloride; THF=Tetrahydrofuran; TLC=Thin layer chromatography; TFA=Trifluoroacetic acid; and TMS=Trimethylsilyl.

Unless described to the contrary for individual compounds, compounds of the examples were analyzed using the following instrumentation: (1) UPLC MS, Waters Acquity-SQD, under the following conditions, (A) column: BEH C-18 reverse phase, 1.7 um, 1.0×50 mm; (B) UPLC: 0.25 ml/min, linear gradient 2% aqueous to 99% organic, where aqueous is acetonitrile:water:TFA [5:95:0.05% (v/v)] and organic is acetonitrile:TFA [100:0.04% (v/v)].; and (C) MS: SQ equipped with an electrospray ionization chamber. (2) High resolving power accurate mass measurements were acquired by use of a Bruker Daltonics 7T Fourier transform ion cyclotron resonance (FTICR) mass spectrometer. Samples were dissolved in acetonitrile:water:acetic acid [50:50:0.1% (v/v)], and ionized by use of electrospray ionization (ESI). External calibration was accomplished with oligomers of polypropylene glycol (PPG, average molecular weight 1000 Da.

With reference to the “General Scheme”, below, in general, compounds of the invention can be prepared by providing the dione compound of Formula A (Step 1) from condensation reaction of an appropriately substituted beta-unsaturated acid of Formula Aa and Meldrum's acid (malonic acid di-isopropionate, Formula Ab), with subsequent cyclization to yield the cyclohexadione intermediate of Formula A.

### General Scheme

### Step 1

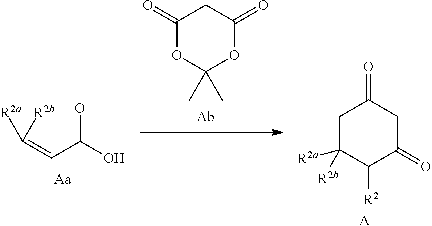

Following Step 1, the dione-intermediate of Formula A is further reacted with carbondisulfide and chloroacetone in the presence of cesium carbonate to produce a lactam/thiopene intermediate which is further reacted with an appropriate alkyl-iodide, or substituted-alkyl iodide (I—R″) to provide the intermediate of Formula B, as shown in Step 2.

### Step 2

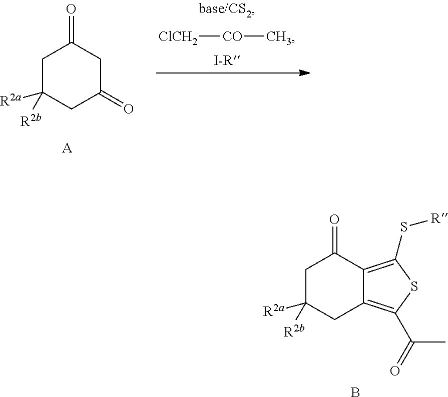

The lactam/thiopene intermediate of Formula B is further reacted in Step 3 to provide the desired substituents on the thiopene ring and may also be further reacted to further modify substituents present on the sulfanyl moiety (—R″) and the lactam ring nitrogen atom.

### Step 3

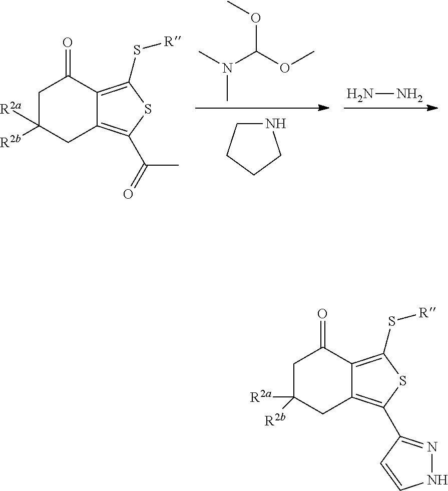

After Step 3, as shown below, the thiol moiety may be oxidized to yield the corresponding sulfonyl compound, which can be derivatized by nucleophilic attack to yield, for example, amine moieties, oxo-moieties, alkyl moieties and similar derivatives available by replacement of the sulfonyl moiety.

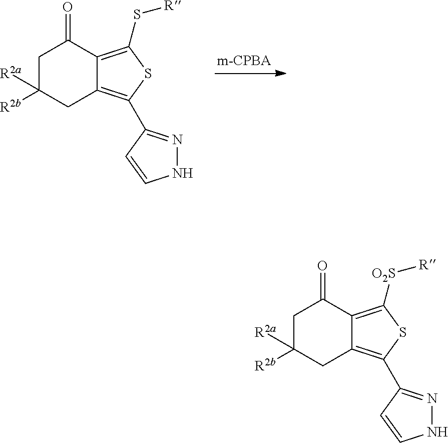

As shown in the Examples below, through appropriate selection of reagents and reaction conditions at various points shown in the general reaction scheme, additional modification can be carried out on various of the intermediate and product compounds described above to provide variously substituted compounds in accordance with the present invention.

Details follow of the synthesis of compounds of this invention made in accordance with this general procedure, which exemplify but do not limit the scope of the invention. Modifications of and departures from the general synthesis scheme, where appropriate, are noted in individual examples where such modifications or departures were made.

As shown in the Examples below, through appropriate selection of reagents and reaction conditions, additional modification can be carried out on the substituents bonded to the sulfur moiety pendent from the thiopene ring, the hexanone ring, and the pyrazole ring.

### Example 1

### 3-(Methylsulfanyl)-1-(1H-pyrazol-3-8/1)-6,7-dihydro-2-benzothiophen-4(5H)-one

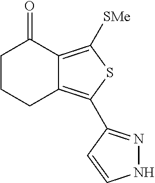 [SMILES: CSc1sc(-c2cc[nH]n2)c2c1C(=O)CCC2]

### Step 1: 1-Acetyl-3-(methylsulfanyl)-6,7-dihydro-2-benzothiophen-4(5H)-one

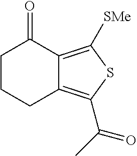

To a mixture of K₂CO₃ (74.0 g, 535 mmol) in DMF (160 mL) was added 1,3-cyclohexane dime (20.0 g, 178 mmol) and the mixture was stirred at RT for 10 min. CS₂ (16.1 mL, 268 mmol) was then added, the reaction mixture was stirred at RT for 10 min and then cooled to 0° C. A solution of chloroacetone (14.2 mL, 178 mmol) in DMF (178 mL) was then added and the reaction mixture was stirred at 0° C. for 1 h. A solution of MeI (12.3 mL, 196 mmol) in DMF (71 mL) was then added and the reaction mixture was allowed to warm to RT and stirred overnight. The mixture was poured into water (2 L) and vigorously stirred for 10 h. The resulting mixture was concentrated in vacuo to remove DMF, partitioned between dichloromethane and water and then filtered. The filtrate was washed four times with water, dried over Na₂SO₄, filtered and concentrated. The product was purified by silica gel chromatography (gradient elution, 0-30% EtOAc in hexanes) to afford an orange solid. The solid was suspended in EtOAc and filtered to yield the title compound as a pale yellow solid. LRMS (ESI) m/z 241.1 [(M+H)⁺; calcd for C₁₁H₁₃O₂S₂: 241].

### Step 2: 3-(Methylsulfanyl)-1-[(2E)-3-(pyrrolidin-1-yl)prop-2-enoyl]-6,7-dihydro-2-benzothiophen-4(5H)-one

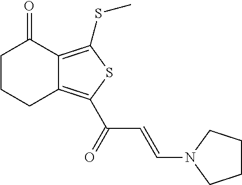 [SMILES: CSc1sc(C(=O)/C=C/N2CCCC2)c2c1C(=O)CCC2]

A mixture of 1-acetyl-3-(methylsulfanyl)-6,7-dihydro-2-benzothiophen-4(5H)-one (5.09 g, 21.2 mmol), dimethylformamide dimethylacetal (28.4 mL, 212 mmol) and pyrrolidine (8.76 mL, 106 mmol) was stirred vigorously at RT for 10 h. The reaction mixture was diluted with dichloromethane and washed three times with water. The organic layer was dried over Na₂SO₄, filtered, concentrated and suspended in EtOAc. The resulting slurry was filtered (Buchner funnel) and the collected solid was washed with EtOAc to yield the title compound, used with no further purification. Additional material was recovered via concentration of the filtrate and purification by silica gel chromatography (gradient elution, 0-5% MeOH in dichloromethane). LRMS (ESI) m/z 322.0 [(M+H)⁺; calcd for C₁₆H₂₀N₂S₂: 322].

### Step 3: 3-(Methylsulfanyl)-1-(1H-pyrazol-3-yl)-6,7-dihydro-2-benzothiophen-4(5H)-one

A mixture of 3-(methylsulfanyl)-1-[(2E)-3-(pyrrolidin-1-yl)prop-2-enoyl]-6,7-dihydro-2-benzothiophen-4(5H)-one (2.20 g, 6.84 mmol) and hydrazine (0.26 mL, 8.21 mmol) in dioxane (17 mL) was heated to 50° C. and stirred for 18 h. The reaction mixture was then charged with additional hydrazine (0.1 eq) and stirred for 18 h, at which time the reaction was shown to be complete by HPLC analysis. The reaction mixture was diluted with EtOAc, filtered and washed with EtOAc to give the title compound as a yellow solid. ¹H NMR (400 MHz, CDCl₃) δ 10.0 (bs, NH), 7.64 (d, J=2.4 Hz, 1H), 6.48 (d, J=2.4 Hz, 1H), 3.01 (t, J=6.2 Hz, 2H), 2.63 (s, 3H), 2.58 (t, J=6.4 Hz, 2H), 2.08 (q, J=6.4 Hz, 2H) Ppm. LRMS (ESI) m/z 265.0 [(M+H)⁺; calcd for C₁₂H₁₃N₂OS₂: 265].

### Example 2

### 3-(Methylsulfonyl)-1-(1H-pyrazol-3-yl)-6,7-dihydro-2-benzothiophen-4(5H)-one

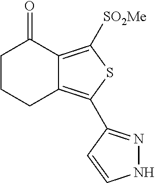 [SMILES: CS(=O)(=O)c1sc(-c2cc[nH]n2)c2c1C(=O)CCC2]

A mixture of 3-(methylsulfanyl)-1-(1H-pyrazol-3-yl)-6,7-dihydro-2-benzothiophen-4(5H)-one (Example 1) (1.00 g, 3.78 mmol) and mCPBA (1.40 g, 5.67 mmol) in dichloromethane (38 mL) was stirred at RT for 3 h. Additional small portions of mCPBA were added at 3 h intervals to drive the reaction to completion by HPLC analysis. The reaction mixture was diluted with dichloromethane and washed with three times with NaHCO₃. The organic extract was dried over Na₂SO₄, filtered and concentrated to afford a pale yellow solid, which was washed with Et₂O and dried to give the title compound. ¹H NMR (400 MHz, CDCl₃) δ 7.69 (d, J=2.6 Hz, 1H), 6.59 (d, J=2.5 Hz, 1H), 3.55 (s, 3H), 3.12 (t, J=6.2 Hz, 2H), 2.69 (t, J=6.6 Hz, 2H), 2.16 (q, J=6.4 Hz, 2H) ppm. LRMS (ESI) m/z 297.0 [(M+H)⁺; calcd for C₁₂H₁₃N₂O₃S₂: 297].

### Example 3

### 3-(Cyclopentylsulfanyl)-1-(1H-pyrazol-3-yl)-6,7-dihydro-2-benzothiophen-4(5H)-one

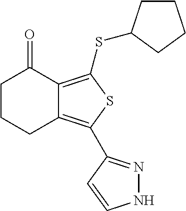 [SMILES: O=C1CCCc2c(-c3cc[nH]n3)sc(SC3CCCC3)c21]

To a solution of cyclopentanethiol (79 μL, 0.74 mmol) in THF (3.7 mL) at 0° C. was added NaH (29.7 mg, 0.74 mmol). The reaction mixture was warmed to RT and a slurry of 3-(methylsulfonyl)-1-(1H-pyrazol-3-yl)-6,7-dihydro-2-benzothiophen-4(5H)-one (Example 2) (110 mg, 0.37 mmol) in THF (3 mL) was added. The reaction was quenched with aqueous NH₄Cl and partitioned between water and dichloromethane. The aqueous layer was extracted three times with dichloromethane and the combined organics were dried over Na₂SO₄, filtered and concentrated. The crude material was purified by silica gel chromatography (gradient elution, 0-5% MeOH/dichloromethane) to afford the title compound as a beige solid. ¹H NMR (400 MHz, CDCl₃) δ 7.64 (d, J=2.2 Hz, 1H), 6.47 (d, J=1.9 Hz, 1H), 3.68 (m, 1H), 2.98 (t, J=6.2 Hz, 2H), 2.57 (t, J=6.4 Hz, 2H), 2.25 (m, 2H), 2.06 (q, J=6.3 Hz, 2H), 1.78 (m, 2H), 1.66 (m, 2H) ppm. LRMS (ESI) m/z 319.1 [(M+H)⁺; calcd for C₁₆H₁₉N₂OS₂: 319].

### Example 4

### 3-(Cyclopentylamino)-1-(1H-pyrazol-3-yl)-6,7-dihydro-2-benzothiophen-4(5H)-one

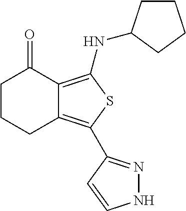 [SMILES: O=C1CCCc2c(-c3cc[nH]n3)sc(NC3CCCC3)c21]

A solution of 3-(methylsulfonyl)-1-(1H-pyrazol-3-yl)-6,7-dihydro-2-benzothiophen-4(5H)-one (Example 2) (259 mg, 0.87 mmol) and cyclopentylamine (149 mg, 1.75 mmol) in DMSO (1.7 mL) was heated in a microwave reactor at 150° C. for 45 min. The reaction mixture was cooled, diluted with dichloromethane and washed three times with water. The organic layer was dried over Na₂SO₄, filtered, concentrated and purified by silica gel chromatography (gradient elution, 5-60% EtOAc/hexanes) to afford the title compound. ¹H NMR (400 MHz, CDCl₃) δ 8.82 (d, J=7.1 Hz, 1H), 7.59 (q, J=1.9 Hz, 1H), 6.41 (bs, NH), 3.76 (m, 1H), 2.88 (t, J=6.2 Hz, 2H), 2.47 (t, J=6.4 Hz, 2H), 2.09 (m, 2H), 2.00 (q, J=6.3 Hz, 2H), 1.77 (m, 2H), 1.67 (m, 4H). LRMS (ESI) m/z 302.1 [(M+H)⁺; calcd for C₁₆H₃₀N₃OS: 302].

### Example 5

### 6,6-Dimethyl-3-(methylsulfanyl)-1-(1H-pyrazol-3-yl)-6,7-dihydro-2-benzothiophen-4(5H)-one

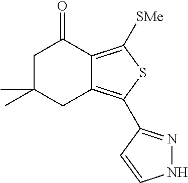 [SMILES: CSc1sc(-c2cc[nH]n2)c2c1C(=O)CC(C)(C)C2]

The title compound was prepared from dimedone using the procedure provided for Example 1. LRMS (ESI) m/z 293.1 [(M+H)⁺; calcd for C₁₄H₁₇N₂OS₂: 293].

### Example 6

### 6,6-Dimethyl-3-(methylsulfonyl)-1-(1H-pyrazol-3-yl)-6,7-dihydro-2-benzothiophen-4(5H)-one

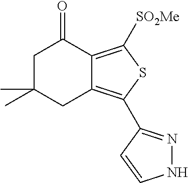 [SMILES: CC1(C)CC(=O)c2c(S(C)(=O)=O)sc(-c3cc[nH]n3)c2C1]

The title compound was prepared from 6,6-dimethyl-3-(methylsulfanyl)-1-(1H-pyrazol-3-yl)-6,7-dihydro-2-benzothiophen-4(5H)-one (Example 5) using the procedure provided for Example 2. ¹H NMR (400 MHz, CDCl₃) δ 10.2 (br s, NH), 7.69 (d, J=2.5 Hz, 1H), 6.59 (d, J=2.6 Hz, 1H), 3.55 (s, 3H), 2.99 (s, 2H), 2.56 (s, 2H), 1.12 (s, 6H) ppm. LRMS (ESI) m/z 325.1 [(M+H)⁺; calcd for C₁₄H₁₇N₂O₃S₂: 325].

### Example 7

### 3-(Cyclopentylsulfanyl)-6,6-dimethyl-1-(1H-pyrazol-3-yl)-6,7-dihydro-2-benzothiophen-4(5H)-one

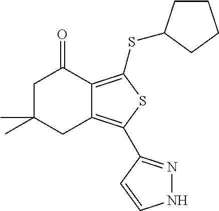 [SMILES: CC1(C)CC(=O)c2c(SC3CCCC3)sc(-c3cc[nH]n3)c2C1]

The title compound was prepared from 6,6-dimethyl-3-(methylsulfonyl)-1-(1H-pyrazol-3-yl)-6,7-dihydro-2-benzothiophen-4(5H)-one (Example 6) using the procedure provided for Example 3. ¹H NMR (400 MHz, CDCl₃) δ 10.0 (bs, NH), 7.64 (d, J=2.4 Hz, 1H), 6.49 (d, J=2.4 Hz, 1H), 3.71 (m, 1H), 2.84 (s, 2H), 2.42 (s, 2H), 2.29 (m, 2H), 1.81 (m, 4H), 1.67 (m, 2H), 1.07 (s, 6H) ppm. LRMS (ESI) m/z 347.1 [(M+H)⁺; calcd for C₁₈H₂₃N₂OS₂: 347].

By following the procedure outlined in Example 3, using 6,6-dimethyl-3-(methylsulfonyl)-1-(1H-pyrazol-3-yl)-6,7-dihydro-2-benzothiophen-4(5H)-one (Example 6) and the appropriate thiol, the following compounds were prepared (Examples 8-34).

| Ex. | Structure | Name | LRMS (M + H)⁺ |
|---|---|---|---|
| 8 | 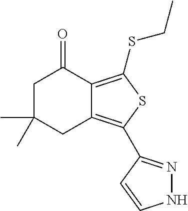 [SMILES: CCSc1sc(-c2cc[nH]n2)c2c1C(=O)CC(C)(C)C2] | 3-(Ethylsulfanyl)-6,6-dimethyl-1- (1H-pyrazol-3-yl)-6,7-dihydro-2- benzothiophen-4(5H)-one | 307.5 |
| 9 | 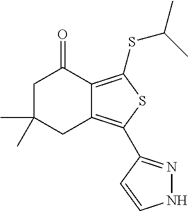 [SMILES: CC(C)Sc1sc(-c2cc[nH]n2)c2c1C(=O)CC(C)(C)C2] | 6,6-Dimethyl-3-(propan-2- ylsulfanyl)-1-(1H-pyrazol-3-yl)-6,7- dihydro-2-benzothiophen-4(5H)-one | 321.5 |
| 10 | 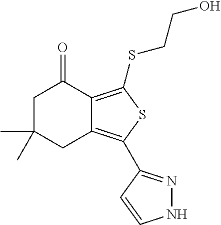 [SMILES: CC1(C)CC(=O)c2c(SCCO)sc(-c3cc[nH]n3)c2C1] | 3-[(2-Hydroxyethyl)sulfanyl]-6,6- dimethyl-1-(1H-pyrazol-3-yl)-6,7- dihydro-2-benzothiophen-4(5H)-one | 323.5 |
| 11 | 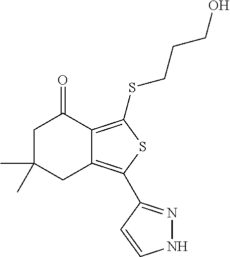 [SMILES: CC1(C)CC(=O)c2c(SCCCO)sc(-c3cc[nH]n3)c2C1] | 3-[(3-Hydroxypropyl)sulfanyl]-6,6- dimethyl-1-(1H-pyrazol-3-yl)-6,7- dihydro-2-benzothiophen-4(5H)-one | 337.5 |
| 12 | 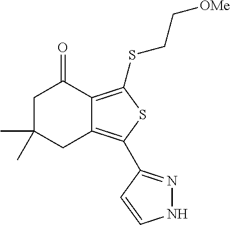 [SMILES: COCCSc1sc(-c2cc[nH]n2)c2c1C(=O)CC(C)(C)C2] | 3-[(2-Methoxyethyl)sulfanyl]-6,6- dimethyl-1-(1H-pyrrazol-3-yl)-6,7- dihydro-2-henzothiophen-4(5H)-one | 337.5 |
| 13 | 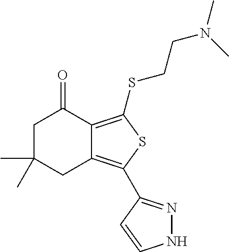 | 3-{[2- (Dimethylamino)ethyl]sulfanyl}-6,6- dimethyl-1-(1H-pyrazol-3-yl)-6,7- dihydro-2-benzothiophen-4(5H)-one | 350.5 |
| 14 | 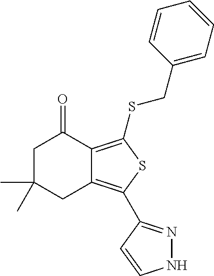 [SMILES: CC1(C)CC(=O)c2c(SCc3ccccc3)sc(-c3cc[nH]n3)c2C1] | 3-(Benzylsulfanyl)-6,6-dimethyl-1- (1H-pyrazol-3-yl)-6,7-dihydro-2- benzothiophen-4(5H)-one | 369.5 |
| 15 | 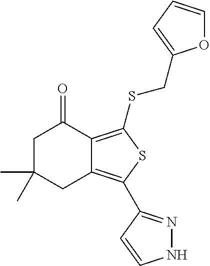 [SMILES: CC1(C)CC(=O)c2c(SCc3ccco3)sc(-c3cc[nH]n3)c2C1] | 3-[(Furan-2-ylmethyl)sulfanyl]-6,6- dimethyl-1-(1H-pyrazol-3-yl)-6,7- dihydro-2-benzothiophen-4(5H)-one | 359.5 |
| 16 | 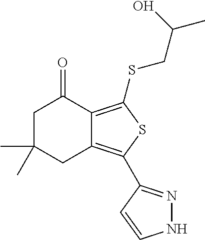 [SMILES: CC(O)CSc1sc(-c2cc[nH]n2)c2c1C(=O)CC(C)(C)C2] | 3-[(2-Hydroxypropyl)sulfanyl]-6,6- dimethyl-1-(1H-pyrazol-3-yl)-6,7- dihydro-2-benzothiophen-4(5H)-one | 337.5 |
| 17 | 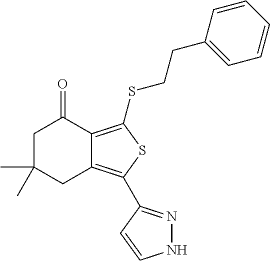 [SMILES: CC1(C)CC(=O)c2c(SCCc3ccccc3)sc(-c3cc[nH]n3)c2C1] | 6,6-Dimethyl-3-[(2- phenylethyl)sulfanyl]-1-(1H-pyrazol- 3-yl)-6,7-dihydro-2-benzothiophen- 4(5H)-one | 383.6 |
| 18 | 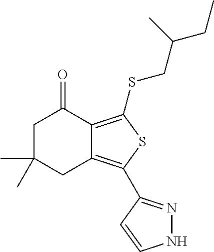 [SMILES: CCC(C)CSc1sc(-c2cc[nH]n2)c2c1C(=O)CC(C)(C)C2] | 6,6-Dimethyl-3-[(2- methylbutyl)sulfanyl]-1-(1H-pyrazol- 3-yl)-6,7-dihydro-2-benzothiophen- 4(5H)-one | 349.5 |
| 19 | 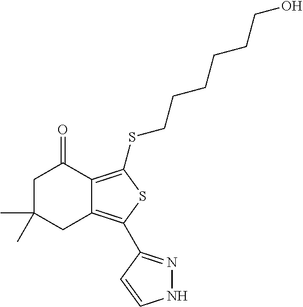 [SMILES: CC1(C)CC(=O)c2c(SCCCCCCO)sc(-c3cc[nH]n3)c2C1] | 3-[(6-Hydroxyhexyl)sulfanyl]-6,6- dimethyl-1-(1H-pyrazol-3-yl)-6,7- dihydro-2-benzothiophen-4(5H)-one | 379.6 |
| 20 | 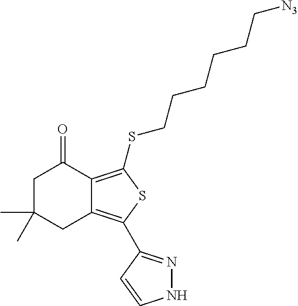 [SMILES: CC1(C)CC(=O)c2c(SCCCCCCN=[N+]=[N-])sc(-c3cc[nH]n3)c2C1] | 3-[(6-Azidohexyl)sulfanyl]-6,6- dimethyl-1-(1H-pyrazol-3-yl)-6,7- dihydro-2-benzothiophen-4(5H)-one | 404.6 |
| 21 | 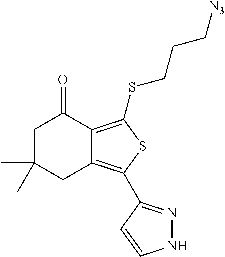 [SMILES: CC1(C)CC(=O)c2c(SCCCN=[N+]=[N-])sc(-c3cc[nH]n3)c2C1] | 3-[(3-Azidopropyl)sulfanyl]-6,6- dimethyl-1-(1H-pyrazol-3-yl)-6,7- dihydro-2-benzothiophen-4(5H)-one | 362.5 |
| 22 | 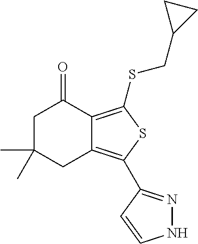 [SMILES: CC1(C)CC(=O)c2c(SCC3CC3)sc(-c3cc[nH]n3)c2C1] | 3-[(Cyclopropylmethyl)sulfanyl]-6,6- dimethyl-1-(1H-pyrazol-3-yl)-6,7- dihydro-2-benzothiophen-4(5H)-one | 333.5 |
| 23 | 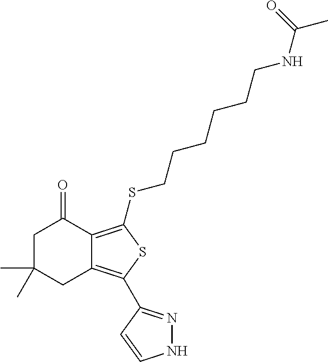 [SMILES: CC(=O)NCCCCCCSc1sc(-c2cc[nH]n2)c2c1C(=O)CC(C)(C)C2] | N-(6-{[5,5-Dimethyl-7-oxo-3-(1H- pyrazol-3-yl)-4,5,6,7-tetrahydro-2- benzothiophen-1- yl]sulfanyl}hexyl)acetamide | 420.6 |
| 24 | 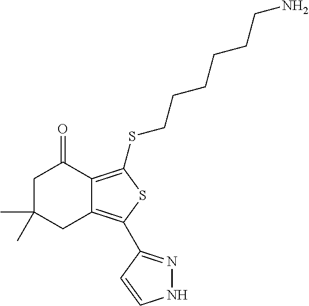 [SMILES: CC1(C)CC(=O)c2c(SCCCCCCN)sc(-c3cc[nH]n3)c2C1] | 3-[(6-Aminohexyl)sulfanyl]-6,6- dimethyl-1-(1H-pyrazol-3-yl)-6,7- dihydro-2-benzothiophen-4(5H)-one | 378.6 |
| 25 | 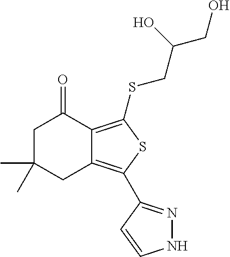 [SMILES: CC1(C)CC(=O)c2c(SCC(O)CO)sc(-c3cc[nH]n3)c2C1] | 3-[(2,3-Dihydroxypropyl)sulfanyl]- 6,6-dimethyl-1-(1H-pyrazol-3-yl)- 6,7-dihydro-2-benzothiophen-4(5H)- one | 353.5 |
| 26 | 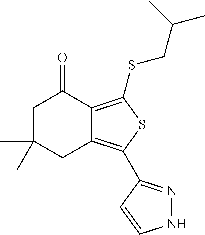 [SMILES: CC(C)CSc1sc(-c2cc[nH]n2)c2c1C(=O)CC(C)(C)C2] | 6,6-Dimethyl-3-[(2- methylpropyl)sulfanyl]-1-(1H- pyrazol-3-yl)-6,7-dihydro-2- benzothiophen-4(5H)-one | 335.5 |
| 27 | 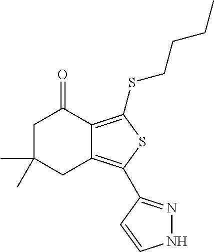 [SMILES: CCCCSc1sc(-c2cc[nH]n2)c2c1C(=O)CC(C)(C)C2] | 3-(Butylsulfanyl)-6,6-dimethyl-1- (1H-pyrazol-3-yl)-6,7-dihydro-2- benzothiophen-4(5H)-one | 335.5 |
| 28 | 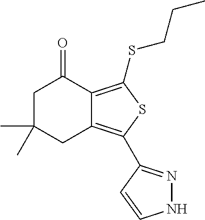 [SMILES: CCCSc1sc(-c2cc[nH]n2)c2c1C(=O)CC(C)(C)C2] | 6,6-Dimethyl-3-(propylsulfanyl)-1- (1H-pyrazol-3-yl)-6,7-dihydro-2- benzothiophen-4(5H)-one | 321.5 |
| 29 | 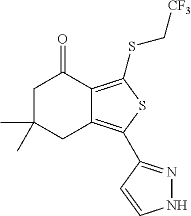 [SMILES: CC1(C)CC(=O)c2c(SCC(F)(F)F)sc(-c3cc[nH]n3)c2C1] | 6,6-Dimethyl-1-(1H-pyrazol-3-yl)-3- [(2,2,2-trifluoroethyl)sulfanyl]-6,7- dihydro-2-benzothiophen-4(5H)-one | 361.4 |
| 30 | 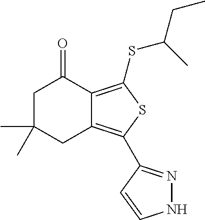 [SMILES: CCC(C)Sc1sc(-c2cc[nH]n2)c2c1C(=O)CC(C)(C)C2] | 3-(Butan-2-ylsulfanyl)-6,6-dimethyl- 1-(1H-pyrazol-3-yl)-6,7-dihydro-2- benzothiophen-4(5H)-one | 335.5 |
| 31 | 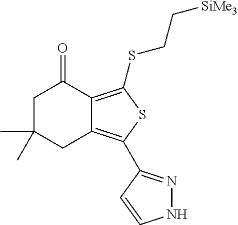 [SMILES: CC1(C)CC(=O)c2c(SCC[Si](C)(C)C)sc(-c3cc[nH]n3)c2C1] | 6,6-Dimethyl-1-(1H-pyrazol-3-yl)-3- {[2-(trimethylsilyl)ethyl]sulfanyl}- 6,7-dihydro-2-benzothiophen-4(5H)- one | 379.6 |
| 32 | 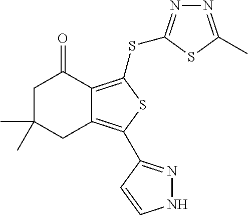 | 6,6-Dimethyl-3-[(5-methyl-1,3,4- thiadiazol-2-yl)sulfanyl]-1-(1H- pyrazol-3-yl)-6,7-dihydro-2- benzothiophen-4(5H)-one | 377.5 |
| 33 | 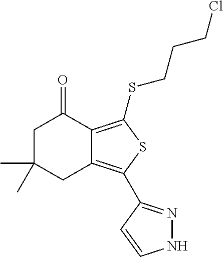 [SMILES: CC1(C)CC(=O)c2c(SCCCCl)sc(-c3cc[nH]n3)c2C1] | 3-[(3-Chloropropyl)sulfanyl]-6,6- dimethyl-1-(1H-pyrazol-3-yl)-6,7- dihydro-2-benzothiophen-4(5H)-one | 355.9 |
| 34 | 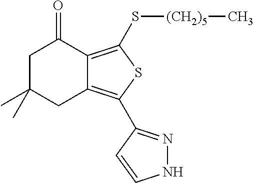 | 3-(Hexylsulfanyl)-6,6-dimethyl-1- (1H-pyrazol-3-yl)-6,7-dihydro-2- benzothiophen-4(5H)-one | 363.6 |

By following the procedure outlined in Example 4, using 6,6-dimethyl-3-(methylsulfonyl)-1-(1H-pyrazol-3-yl)-6,7-dihydro-2-benzothiophen-4(5H)-one (Example 6) and the appropriate amine, the following compounds can be prepared (Examples 35-51).

| Ex. | Structure | Name | LRMS (M + H)⁺ |
|---|---|---|---|
| 35 | 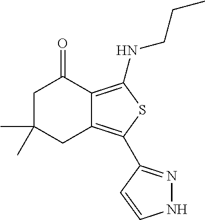 [SMILES: CCCNc1sc(-c2cc[nH]n2)c2c1C(=O)CC(C)(C)C2] | 6,6-Dimethyl-3-(propylamino)-1- (1H-pyrazol-3-yl)-6,7-dihydro-2- benzothiophen-4(5H)-one | 304.4 |
| 36 | 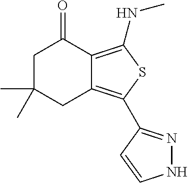 [SMILES: CNc1sc(-c2cc[nH]n2)c2c1C(=O)CC(C)(C)C2] | 6,6-Dimethyl-3-(methylamino)-1- (1H-pyrazol-3-yl)-6,7-dihydro-2- benzothiophen-4(5H)-one | 276.4 |
| 37 | 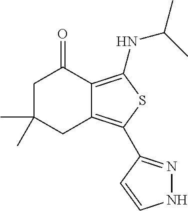 [SMILES: CC(C)Nc1sc(-c2cc[nH]n2)c2c1C(=O)CC(C)(C)C2] | 6,6-Dimethyl-3-(propan-2-ylamino)- 1-(1H-pyrazol-3-yl)-6,7-dihydro-2- benzothiophen-4(5H)-one | 304.4 |
| 38 | 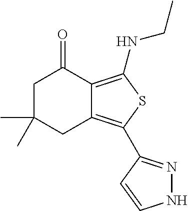 [SMILES: CCNc1sc(-c2cc[nH]n2)c2c1C(=O)CC(C)(C)C2] | 3-(Ethylamino)-6,6-dimethyl-1-(1H- pyrazol-3-yl)-6,7-dihydro-2- benzothiophen-4(5H)-one | 290.4 |
| 39 | 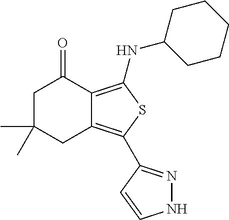 [SMILES: CC1(C)CC(=O)c2c(NC3CCCCC3)sc(-c3cc[nH]n3)c2C1] | 3-(Cyclohexylaminn)-6,6-dimethyl-1- (1H-pyrazol-3-yl)-6,7-dihydro-2- benzothiophen-4(5H)-one | 344.5 |
| 40 | 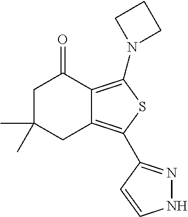 [SMILES: CC1(C)CC(=O)c2c(N3CCC3)sc(-c3cc[nH]n3)c2C1] | 3-(Azetidin-1-yl)-6,6-dimethyl-1- (1H-pyrazol-3-yl)-6,7-dihydro-2- benzothiophen-4(5H)-one | 302.4 |
| 41 | 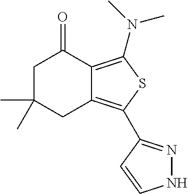 [SMILES: CN(C)c1sc(-c2cc[nH]n2)c2c1C(=O)CC(C)(C)C2] | 3-(Dimethylamino)-6,6-dimethyl-1- (1H-pyrazol-3-yl)-6,7-dihydro-2- benzothiophen-4(5H)-one | 290.4 |
| 42 | 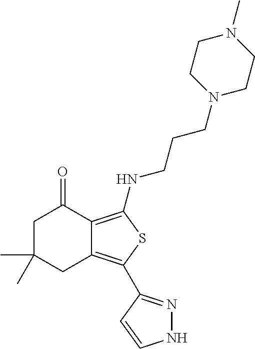 [SMILES: CN1CCN(CCCNc2sc(-c3cc[nH]n3)c3c2C(=O)CC(C)(C)C3)CC1] | 6,6-Dimethyl-3-{[3-(4- methylpiperazin-1-yl)propyl]amino}- 1-(1H-pyrazol-3-yl)-6,7-dihydro-2- benzothiophen-4(5H)-one | 402.6 |
| 43 | 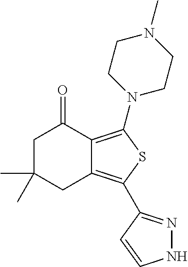 [SMILES: CN1CCN(c2sc(-c3cc[nH]n3)c3c2C(=O)CC(C)(C)C3)CC1] | 6,6-Dimethyl-3-(4-methylpiperazin- 1-yl)-1-(1H-pyrazol-3-yl)-6,7- dihydro-2-benzothiophen-4(5H)-one | 345.5 |
| 44 | 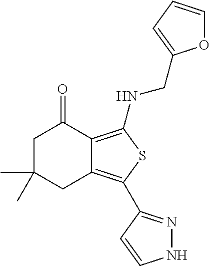 [SMILES: CC1(C)CC(=O)c2c(NCc3ccco3)sc(-c3cc[nH]n3)c2C1] | 3-[(Furan-2-ylmethyl)amino]-6,6- dimethyl-1-(1H-pyrazol-3-yl)-6,7- dihydro-2-benzothiophen-4(5H)-one | 342.4 |
| 45 | 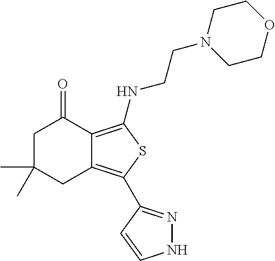 [SMILES: CC1(C)CC(=O)c2c(NCCN3CCOCC3)sc(-c3cc[nH]n3)c2C1] | 6,6-Dimethyl-3-{[2-(morpholin-4- yl)ethyl]amino}-1-(1H-pyrazol-3-yl)- 6,7-dihydro-2-benzothiophen-4(5H)- one | 375.5 |
| 46 | 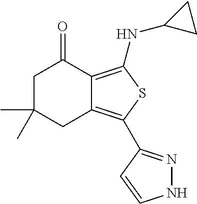 [SMILES: CC1(C)CC(=O)c2c(NC3CC3)sc(-c3cc[nH]n3)c2C1] | 3-(Cyclopropylamino)-6,6-dimethyl- 1-(1H-pyrazol-3-yl)-6,7-dihydro-2- benzothiophen-4(5H)-one | 302.4 |
| 47 | 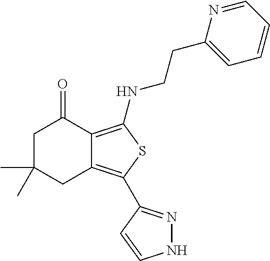 [SMILES: CC1(C)CC(=O)c2c(NCCc3ccccn3)sc(-c3cc[nH]n3)c2C1] | 6,6-Dimethyl-1-(1H-pyrazol-3-yl)-3- {[2-(pyridin-2-yl)ethyl]amino}-6,7- dihydro-2-benzothiophen-4(5H)-one | 367.5 |
| 48 | 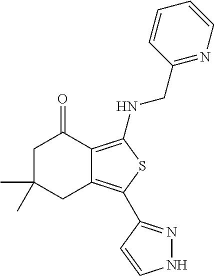 [SMILES: CC1(C)CC(=O)c2c(NCc3ccccn3)sc(-c3cc[nH]n3)c2C1] | 6,6-Dimethyl-1-(1H-pyrazol-3-yl)-3- [(pyridin-2-ylmethyl)amino]-6,7- dihydro-2-benzothiophen-4(5H)-one | 353.5 |
| 49 | 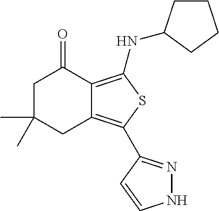 [SMILES: CC1(C)CC(=O)c2c(NC3CCCC3)sc(-c3cc[nH]n3)c2C1] | 3-(Cyclopentylamino)-6,6-dimethyl- 1-(1H-pyrazol-3-yl)-6,7-dihydro-2- benzothiophen-4(5H)-one | 330.5 |
| 50 | 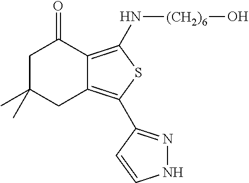 | 3-[(6-Hydroxyhexyl)amino]-6,6- dimethyl-1-(1H-pyrazol-3-yl)-6,7- dihydro-2-benzothiophen-4(5H)-one | 362.5 |
| 51 | 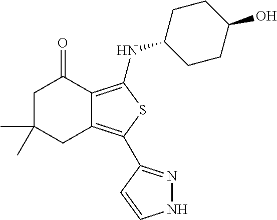 [SMILES: CC1(C)CC(=O)c2c(N[C@H]3CC[C@H](O)CC3)sc(-c3cc[nH]n3)c2C1] | 3-[(trans-4- Hydroxycyclohexyl)amino]-6,6- dimethyl-1-(1H-pyrazol-3-yl)-6,7- dihydro-2-benzothiophen-4(5H)-one | 360.5 |

### Example 52

### 3-(Cyclopentyloxy)-6,6-dimethyl-1-(1H-pyrazol-3-yl)-6,7-dihydro-2-benzothiophen-4(5H)-one

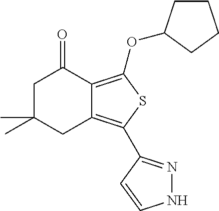 [SMILES: CC1(C)CC(=O)c2c(OC3CCCC3)sc(-c3cc[nH]n3)c2C1]

To a solution of cyclopentanol (22 μL, 0.25 mmol) in DMSO (0.25 mL) was added NaH (10 mg, 0.25 mmol). The reaction mixture was stirred for 10 min and 6,6-dimethyl-3-(methylsulfonyl)-1-(1H-pyrazol-3-yl)-6,7-dihydro-2-benzothiophen-4(5H)-one (Example 6) (40 mg, 0.12 mmol) was added. The reaction mixture was heated to 60° C., stirred for 30 min and cooled to RT. The mixture was purified by preparative reverse-phase HPLC (gradient elution, 15-90% MeCN/water; 0.05% TEA). The fractions containing the desired product were concentrated, partitioned between aqueous NaHCO₃ and dichloromethane. The organic layer was dried over Na₂SO₄, filtered and concentrated to give the title compound. ¹H NMR (400 MHz, CDCl₃) δ 7.62 (d, J=2.1 Hz, 1H), 6.46 (br s, 1H), 4.84 (q, J=2.8 Hz, 1H), 2.80 (s, 2H), 2.35 (s, 2H), 1.87-2.07 (m, 8H), 1.06 (s, 6H) ppm. LRMS (ESI) m/z 331.1 [(M+H)⁺; calcd for C₁₈H₂₃N₂O₂S: 331].

By following the procedure outlined in Example 52, using the appropriate alcohol, the following compounds were prepared (Examples 53-55).

| Ex. | Structure | Name | LRMS (M + H)⁺ |
|---|---|---|---|
| 53 | 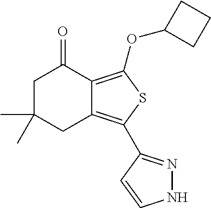 [SMILES: CC1(C)CC(=O)c2c(OC3CCC3)sc(-c3cc[nH]n3)c2C1] | 3-(Cyclobutylexy)-6,6-dimethyl-1- (1H-pyrazol-3-yl)-6,7-dihydro-2- benzothiophen-4(5H)-one | 317.4 |
| 54 | 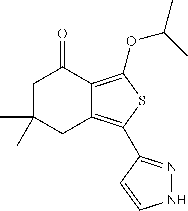 [SMILES: CC(C)Oc1sc(-c2cc[nH]n2)c2c1C(=O)CC(C)(C)C2] | 6,6-Dimethyl-3-(propan-2-yloxy)-1- (1H-pyrazol-3-yl)-6,7-dihydro-2- benzothiophen-4(5H)-one | 305.4 |
| 55 | 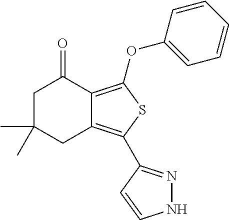 [SMILES: CC1(C)CC(=O)c2c(Oc3ccccc3)sc(-c3cc[nH]n3)c2C1] | 6,6-Dimethyl-3-phenoxy-1-(1H- pyrazol-3-yl)-6,7-dihydro-2- benzothiophen-4(5H)-one | 339.4 |

### Example 56

### 6,6-Dimethyl-3-propyl-1-(1H-pyrazol-3-yl)-6,7-dihydro-2-benzothiophen-4(5H)-one

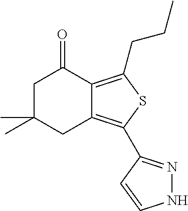 [SMILES: CCCc1sc(-c2cc[nH]n2)c2c1C(=O)CC(C)(C)C2]

To a solution of propylmagnesium chloride (154 μL, 0.31 mmol, 2 M in THF) in THF (0.7 mL) at −78° C. was added CuBr-dimethyl sulfide complex (1.58 mg, 7.7 μmol) and trimethylchlorosilane (33.5 mg, 0.31 mmol). To this solution was added 6,6-dimethyl-3-(methylsulfonyl)-1-(1H-pyrazol-3-yl)-6,7-dihydro-2-benzothiophen-4(5H)-one (Example 6) (25 mg, 0.08 mmol) in THF (0.3 mL). The reaction mixture was warmed to −50° C. and stirred for 1 h, then quenched with aqueous NH₄Cl and extracted with ether. The organic layer was dried over Na₂SO₄, filtered, concentrated and purified by silica gel chromatography (gradient elution, 0-30% EtOAc in hexane) to give the title compound. ¹H NMR (400 MHz, CDCl₃) δ 7.63 (s, 1H), 6.48 (s, 1H), 3.21 (t, J=7.5 Hz, 2H), 2.83 (s, 2H), 2.38 (s, 2H), 1.70 (hextet, J=7.5 Hz, 2H), 1.03 (s, 6H), 0.99 (t, J=7.5 Hz, 3H). LRMS (ESI) m/z 289.1 [(M+H)⁺; calcd for C₁₆H₂₁N₂OS: 2891

### Example 57

### 3,3-(Cyclopentylsulfanyl)-6,6-dimethyl-4-oxo-4,5,6,7-tetrahydro-2-benzothiophen-1-yl]-N-(propan-2-yl)-1H-pyrazole-1-carboxamide

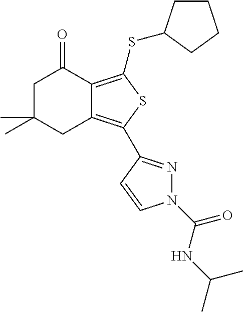 [SMILES: CC(C)NC(=O)n1ccc(-c2sc(SC3CCCC3)c3c2CC(C)(C)CC3=O)n1]

To a solution of 3-(cyclopentylsulfanyl)-6,6-dimethyl-1-(1H-pyrazol-3-yl)-6,7-dihydro-2-benzothiophen-4(5H)-one (Example 7) (40 mg, 0.12 mmol) in dichloromethane (1.15 mL) at RT was added isopropyl isocyanate (10.3 mg, 0.12 mmol). The reaction mixture was stirred at RT for 15 h, concentrated and purified by silica gel chromatography (gradient elution, 0.40% EtOAc in hexane) to give the title compound. ¹H NMR (400 MHz, CDCl₃) δ 8.27 (d, J=2.7 Hz, 1H), 6.91 (d, J=8.1 Hz, 1H), 6.54 (d, J=2.7 Hz, 1H), 4.21-4.10 (m, 1H), 3.77-3.69 (m, 1H), 2.83 (s, 2H), 2.43 (s, 2H), 2.35-2.25 (m, 2H), 1.88-1.75 (m, 4H), 1.74-1.63 (m, 2H), 1.33 (d, J=6.6 Hz, 6H), 1.07 (s, 6H) ppm. LRMS (ESI) m/z 432.1 [(M+H)⁺; calcd for C₂₂H₃₀N₃O₂S₂: 432].

By following the procedure outlined in Example 57, using the appropriate isocyanate, the following compounds could be prepared (Examples 58-72).

| Ex. | Structure | Name | LRMS (M + H)⁺ |
|---|---|---|---|
| 58 | 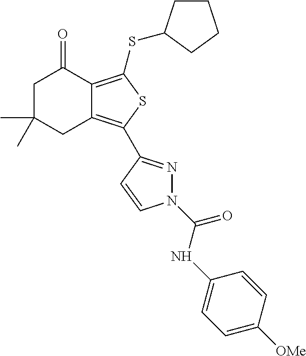 [SMILES: COc1ccc(NC(=O)n2ccc(-c3sc(SC4CCCC4)c4c3CC(C)(C)CC4=O)n2)cc1] | 3-[3-(Cyclopentylsulfanyl)-6,6- dimethyl-4-oxo-4,5,6,7-tetrahydro- 2-benzothiophen-1-yl]-N-(4- methoxyphenyl)-1H-pyrazole-1- carboxamide | 496.1 |
| 59 | 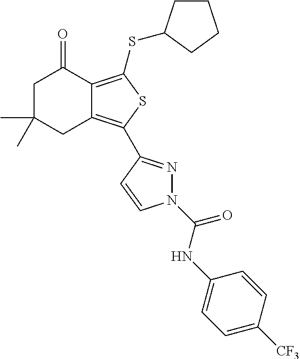 [SMILES: CC1(C)CC(=O)c2c(SC3CCCC3)sc(-c3ccn(C(=O)Nc4ccc(C(F)(F)F)cc4)n3)c2C1] | 3-[3-(Cyclopentylsulfanyl)-6,6- dimethyl-4-oxo-4,5,6,7-tetrahydro- 2-benzothiophen-1-yl]-N-[4- (trifluoromethyl)phenyl]-1H- pyrazole-1-carboxamide | 534.2 |
| 60 | 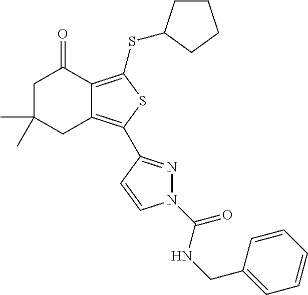 [SMILES: CC1(C)CC(=O)c2c(SC3CCCC3)sc(-c3ccn(C(=O)NCc4ccccc4)n3)c2C1] | N-benzyl-3-[3- (Cyclopentylsulfanyl)-6,6-dimethyl- 4-oxo-4,5,6,7-tetrahydro-2- benzothiophen-1-yl]-1H-pyrazole-1- carboxamide | 480.1 |
| 61 | 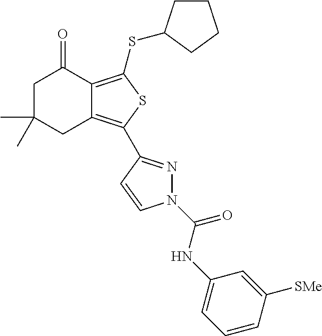 [SMILES: CSc1cccc(NC(=O)n2ccc(-c3sc(SC4CCCC4)c4c3CC(C)(C)CC4=O)n2)c1] | 3-[3-(Cyclopentylsulfanyl)-6,6- dimethyl-4-oxo-4,5,6,7-tetrahydro- 2-benzothiophen-1-yl]-N-[3- (methylsulfanyl)phenyl]-1H- pyrazole-1-carboxamide | 512.1 |
| 62 | 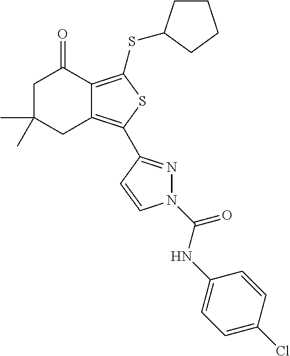 [SMILES: CC1(C)CC(=O)c2c(SC3CCCC3)sc(-c3ccn(C(=O)Nc4ccc(Cl)cc4)n3)c2C1] | N-(4-Chlorophenyl)-3-[3- (cyclopentylsulfanyl)-6,6-dimethyl- 4-oxo-4,5,6,7-tetrahydro-2- benzothiophen-1-yl]-1H-pyrazole-1- carboxamide | 501.1 |
| 63 | 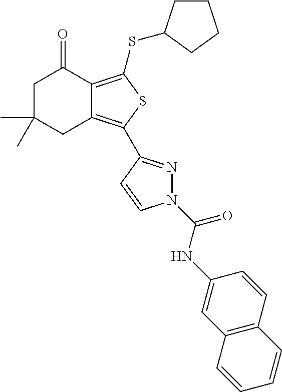 [SMILES: CC1(C)CC(=O)c2c(SC3CCCC3)sc(-c3ccn(C(=O)Nc4ccc5ccccc5c4)n3)c2C1] | 3-[3-(Cyclopentylsulfanyl)-6,6- dimethyl-4-oxo-4,5,6,7-tetrahydro- 2-benzothiophen-1-yl]-N- (naphthalen-2-yl)-1H-pyrazole-1- carboxamide | 516.1 |
| 64 | 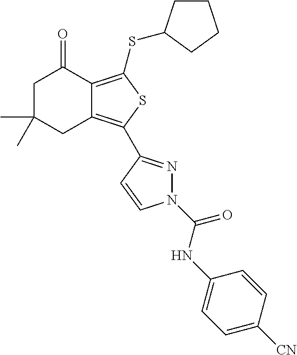 [SMILES: CC1(C)CC(=O)c2c(SC3CCCC3)sc(-c3ccn(C(=O)Nc4ccc(C#N)cc4)n3)c2C1] | N-(4-Cyanophenyl)-3-[3- (cyclopentylsulfanyl)-6,6-dimethyl- 4-oxo-4,5,6,7-tetrahydro-2- benzothiophen-1-yl]-1H-pyrazole-1- carboxamide | 491.1 |
| 65 | 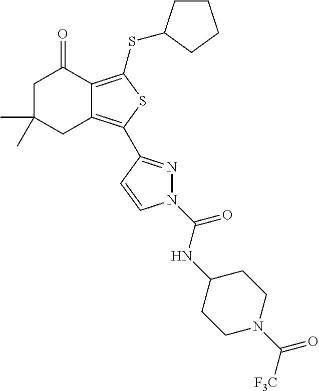 [SMILES: CC1(C)CC(=O)c2c(SC3CCCC3)sc(-c3ccn(C(=O)NC4CCN(C(=O)C(F)(F)F)CC4)n3)c2C1] | 3-[3-(Cyclopentylsulfanyl)-6,6- dimethyl-4-oxo-4,5,6,7-tetrahydro- 2-benzothiophen-1-yl]-N-[1- (trifluoroacetyl)piperidin-4-yl]-1H- pyrazole-1-carboxamide | 569.2 |
| 66 | 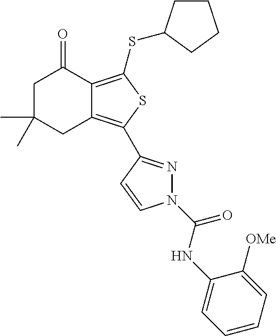 [SMILES: COc1ccccc1NC(=O)n1ccc(-c2sc(SC3CCCC3)c3c2CC(C)(C)CC3=O)n1] | 3-[3-(Cyclopentylsulfanyl)-6,6- dimethyl-4-oxo-4,5,6,7-tetrahydro- 2-benzothiophen-1-yl]-N-(2- methoxyphenyl)-1H-pyrazole-1- carboxamide | 496.1 |
| 67 | 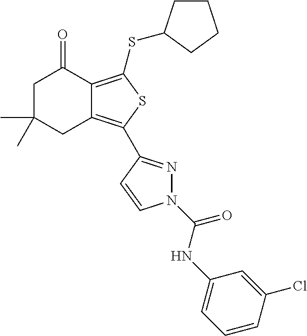 [SMILES: CC1(C)CC(=O)c2c(SC3CCCC3)sc(-c3ccn(C(=O)Nc4cccc(Cl)c4)n3)c2C1] | N-(3-Chlorophenyl)-3-[3- (cyclopentylsulfanyl)-6,6-dimethyl- 4-oxo-4,5,6,7-tetrahydro-2- benzothiophen-1-yl]-1H-pyrazole-1- carboxamide | 501.1 |
| 68 | 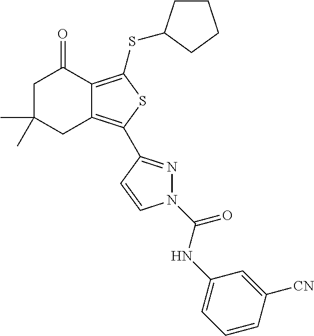 [SMILES: CC1(C)CC(=O)c2c(SC3CCCC3)sc(-c3ccn(C(=O)Nc4cccc(C#N)c4)n3)c2C1] | N-(3-Cyanophenyl)-3-[3- (cyclopentylsulfanyl)-6,6-dimethyl- 4-oxo-4,5,6,7-tetrahydro-2- benzothiophen-1-yl]-1H-pyrazole-1- carboxamide | 491.1 |
| 69 | 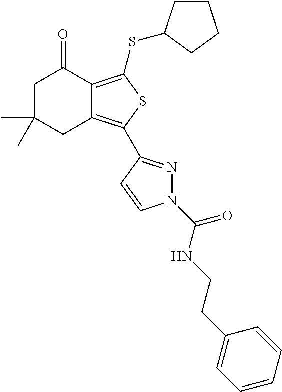 [SMILES: CC1(C)CC(=O)c2c(SC3CCCC3)sc(-c3ccn(C(=O)NCCc4ccccc4)n3)c2C1] | 3-[3-(Cyclopentylsulfanyl)-6,6- dimethyl-4-oxo-4,5,6,7-tetrahydro- 2-benzothiophen-1-yl]-N-(2- phenylethyl)-1H-pyrazole-1- carboxamide | 494.1 |
| 70 | 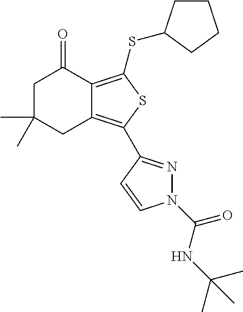 [SMILES: CC1(C)CC(=O)c2c(SC3CCCC3)sc(-c3ccn(C(=O)NC(C)(C)C)n3)c2C1] | N-tert-Butyl-3-[3- (cyclopentylsulfanyl)-6,6-dimethyl- 4-oxo-4,5,6,7-tetrahydro-2- benzothiophen-1-yl]-1H-pyrazole-1- carboxamide | 446.1 |
| 71 | 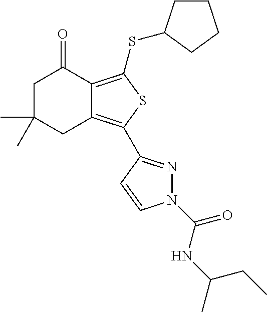 [SMILES: CCC(C)NC(=O)n1ccc(-c2sc(SC3CCCC3)c3c2CC(C)(C)CC3=O)n1] | N-(Butan-2-yl)-3-[3- (cyclopentylsulfanyl)-6,6-dimethyl- 4-oxo-4,5,6,7-tetrahydro-2- benzothiophen-1-yl]-1H-pyrazole-1- carboxamide | 446.1 |
| 72 | 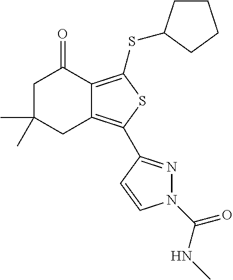 [SMILES: CNC(=O)n1ccc(-c2sc(SC3CCCC3)c3c2CC(C)(C)CC3=O)n1] | 3-[3-(Cyclopentylsulfanyl)-6,6- dimethyl-4-oxo-4,5,6,7-tetrahydro- 2-benzothiophen-1-yl]-N-methyl- 1H-pyrazole-1-carboxamide | 404.1 |

By following the procedure outlined in Example 57, using 3-(Cyclopentylamino)-6,6-dimethyl-1-(1H-pyrazol-3-yl)-6,7-dihydro-2-benzothiophen-4(5H)-one (Example 49) and the appropriate isocyanate, the following compounds could be prepared (Examples 73-75).

| Ex. | Structure | Name | LRMS (M + H)⁺ |
|---|---|---|---|
| 73 | 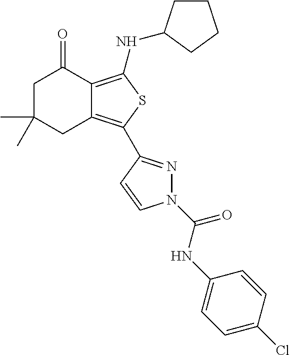 [SMILES: CC1(C)CC(=O)c2c(NC3CCCC3)sc(-c3ccn(C(=O)Nc4ccc(Cl)cc4)n3)c2C1] | N-(4-Chlorophenyl)-3-[3- (cyclopentylamino)-6,6-dimethyl-4- oxo-4,5,6,7-tetrahydro-2- benzothiophen-1-yl]-1H-pyrazole-1- carboxamide | 484.1 |
| 74 | 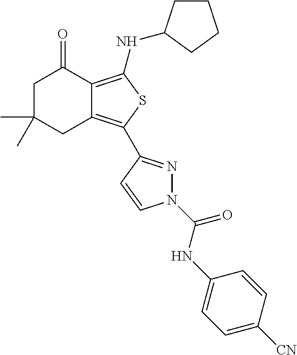 [SMILES: CC1(C)CC(=O)c2c(NC3CCCC3)sc(-c3ccn(C(=O)Nc4ccc(C#N)cc4)n3)c2C1] | N-(4-Cyanophenyl)-3-[3- (cyclopentylamino)-6,6-dimethyl-4- oxo-4,5,6,7-tetrahydro-2- benzothiophen-1-yl]-1H-pyrazole-1- carboxamide | 474.1 |
| 75 | 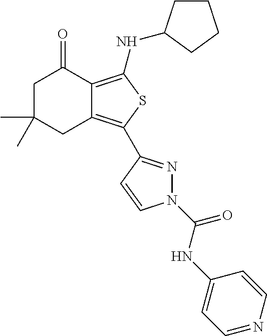 [SMILES: CC1(C)CC(=O)c2c(NC3CCCC3)sc(-c3ccn(C(=O)Nc4ccncc4)n3)c2C1] | 3-[3-(Cyclopentylamino)-6,6- dimethyl-4-oxo-4,5,6,7-tetrahydro- 2-benzothiophen-1-yl]-N-(pyridin-4- yl)-1H-pyrazole-1-carboxamide | 450.1 |

### Example 76

### 1-(1-Butanoyl-1H-pyrazol-3-yl)-3-(cyclopentylsulfanyl)-6,6-dimethyl-6,7-dihydro-2-benzothiophen-4(5H)-one

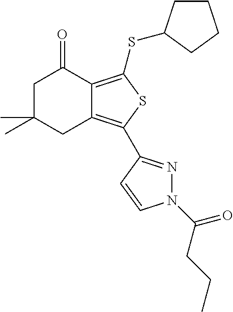 [SMILES: CCCC(=O)n1ccc(-c2sc(SC3CCCC3)c3c2CC(C)(C)CC3=O)n1]

To a solution of 3-(cyclopentylsulfanyl)-6,6-dimethyl-1-(1H-pyrazol-3-yl)-6,7-dihydro-2-benzothiophen-4(5H)-one (Example 7) (40 mg, 0.12 mmol) in dichloromethane (0.3 mL) at RT was added triethylamine (32 μL, 0.23 mmol) and butyryl chloride (12.3 mg, 0.12 mmol). The reaction mixture was stirred at RT for 15 h, diluted with dichloromethane, washed with water and brine and concentrated. The crude product was purified by silica gel chromatography (gradient elution, 0-60% EtOAc in hexane) to give the title compound. ¹H NMR (400 MHz, CDCl₃): δ 8.30 (d, J=2.9 Hz, 1H), 6.60 (d, J=2.9 Hz, 1H), 3.77-3.69 (m, 1H), 3.15 (t, J=7.3 Hz, 3H), 2.88 (s, 2H), 2.46 (s, 2H), 2.30 (m, 2H), 1.91-4.74 (m, 6H), 1.73-1.63 (m, 2H), 1.08 (m, 9H) ppm. LRMS (ESI) m/z 417.0 [(M+H)⁺; calcd for C₂₂H₂₉N₂O₂S₂: 417].

By following the procedure outlined in Example 76, using the appropriate acid chloride, the following compounds could be prepared (Examples 77-84).

| Ex. | Structure | Name | LRMS (M + H)⁺ |
|---|---|---|---|
| 77 | 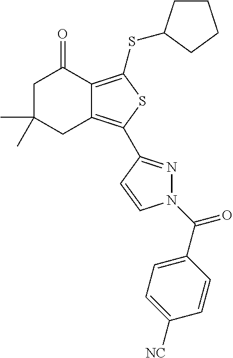 [SMILES: CC1(C)CC(=O)c2c(SC3CCCC3)sc(-c3ccn(C(=O)c4ccc(C#N)cc4)n3)c2C1] | 4-({3-[3-(Cyclopentylsulfanyl)-6,6- dimethyl-4-oxo-4,5,6,7-tetrahydro- 2-benzothiophen-1-yl]-1H-pyrazol- 1-yl}carbonyl)benzonitrile | 476.1 |
| 78 | 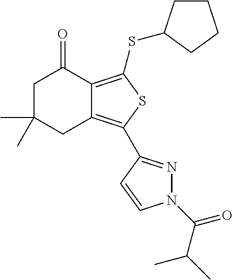 [SMILES: CC(C)C(=O)n1ccc(-c2sc(SC3CCCC3)c3c2CC(C)(C)CC3=O)n1] | 3-(Cyclopentylsulfanyl)-6,6- dimethyl-1-[1-(2- methylpropanoyl)-1H-pyrazol-3- yl]-6,7-dihydro-2-benzothiophen- 4(5H)-one | 417.1 |
| 79 | 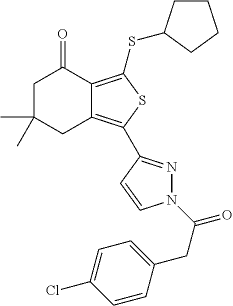 [SMILES: CC1(C)CC(=O)c2c(SC3CCCC3)sc(-c3ccn(C(=O)Cc4ccc(Cl)cc4)n3)c2C1] | 1-{1-[(4-Chlorophenyl)acetyl]-1H- pyrazol-3-yl}-3- (cyclopentylsulfanyl)-6,6-dimethyl- 6,7-dihydro-2-benzothiophen- 4(5H)-one | 500.1 |
| 80 | 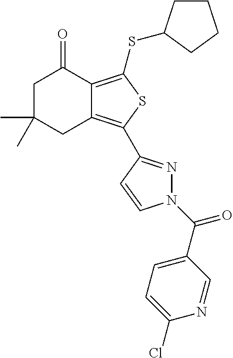 [SMILES: CC1(C)CC(=O)c2c(SC3CCCC3)sc(-c3ccn(C(=O)c4ccc(Cl)nc4)n3)c2C1] | 1-{1-[(6-Chloropyridin-3- yl)carbonyl]-1H-pyrazol-3-yl}-3- (cyclopentylsulfanyl)-6,6-dimethyl- 6,7-dihydro-2-benzothiophen- 4(5H)-one | 487.1 |
| 81 | 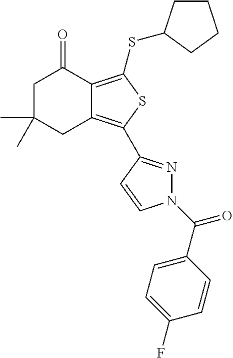 [SMILES: CC1(C)CC(=O)c2c(SC3CCCC3)sc(-c3ccn(C(=O)c4ccc(F)cc4)n3)c2C1] | 3-(Cyclopentylsulfanyl)-1-{1-[(4- fluorophenyl)carbonyl]-1H- pyrazol-3-yl}-6,6-dimethyl-6,7- dihydro-2-benzothiophen-4(5H)- one | 469.1 |
| 82 | 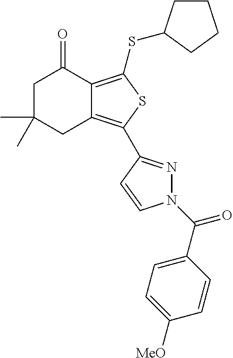 [SMILES: COc1ccc(C(=O)n2ccc(-c3sc(SC4CCCC4)c4c3CC(C)(C)CC4=O)n2)cc1] | 3-(Cyclopentylsulfanyl)-1-{1-[(4- methoxyphenyl)carbonyl]-1H- pyrazol-3-yl}-6,6-dimethyl-6,7- dihydro-2-benzothiophen-4(5H)- one | 481.1 |
| 83 | 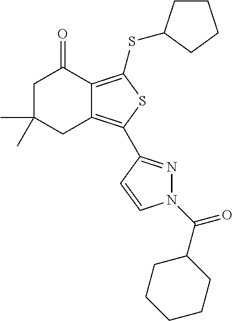 [SMILES: CC1(C)CC(=O)c2c(SC3CCCC3)sc(-c3ccn(C(=O)C4CCCCC4)n3)c2C1] | 1-[1-(Cyclohexylcarbonyl)-1H- pyrazol-3-yl]-3- (cyclopentylsulfanyl)-6,6-dimethyl- 6,7-dihydro-2-benzothiophen- 4(5H)-one | 457.1 |
| 84 | 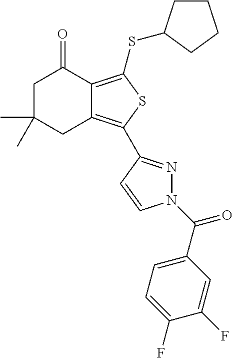 [SMILES: CC1(C)CC(=O)c2c(SC3CCCC3)sc(-c3ccn(C(=O)c4ccc(F)c(F)c4)n3)c2C1] | 3-(Cyclopentylsulfanyl)-1-{1-[(3,4- difluorophenyl)carbonyl]-1H- pyrazol-3-yl}-6,6-dimethyl-6,7- dihydro-2-benzothiophen-4(5H)- one | 487.1 |

### Example 85

### tert-Butyl 3-[3-(methylsulfanyl)-4-oxo-4,5,6,7-tetrahydro-2-benzothiophen-1-yl]-1H-pyrazole-1-carboxylate

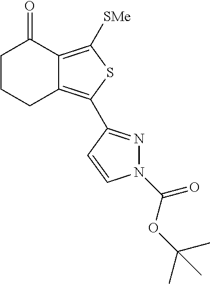 [SMILES: CSc1sc(-c2ccn(C(=O)OC(C)(C)C)n2)c2c1C(=O)CCC2]

To a mixture of 3-(methylsulfanyl)-1-(1H-pyrazol-3-yl)-6,7-dihydro-2-benzothiophen-4(5H)-one (Example 1) (263 mg, 1.00 mmol) and di-tert-butyl dicarbonate (261 mg, 1.20 mmol) in dichloromethane (5 mL) was added triethylamine (278 μL, 1.20 mmol). The reaction mixture was heated to 50° C. and stirred for 14 h. The reaction mixture was then diluted with dichloromethane, washed three times with water, dried over Na₂SO₄, filtered and concentrated. The crude product was purified by silica gel chromatography (gradient elution, 0-30% EtOAc in hexane) to give the title compound. ¹H NMR (400 MHz, CDCl₃) δ 8.09 (d, J=2.8 Hz, 1H), 6.54 (d, J=2.8 Hz, 1H), 3.05 (t, J=6.2 Hz, 2H), 2.63 (s, 3H), 2.57 (t, J=6.4 Hz, 2H), 1.67 (s, 9H) ppm. LRMS (ESI) m/z 365.1 [(M+H)⁺; calcd for C₁₇H₂₁N₂O₃S₂: 365].

### Example 86

### N-(4-chlorophenyl)-3-[3-(cyclopentylsulfanyl)-4-oxo-4,5,6,7-tetrahydro-2-benzothiophen-1-yl]-1H-pyrazole-1-carboxamide

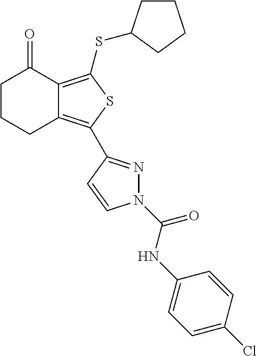 [SMILES: O=C1CCCc2c(-c3ccn(C(=O)Nc4ccc(Cl)cc4)n3)sc(SC3CCCC3)c21]

The title compound was prepared by following the procedure outlined in Example 57, using 3-(cyclopentylsulfanyl)-1-(1H-pyrazol-3-yl)-6,7-dihydro-2-benzothiophen-4(5H)-one (Example 3) and 4-chlorophenyl isocyanate. ¹H NMR (400 MHz, CDCl₃): δ 9.00 (s, 1H), 8.33 (d, J=2.8 Hz, 1H), 7.60 (d, J=9.1 Hz 2H), 7.37 (d, J=9.1 Hz, 2H), 6.62 (d, J=2.8 Hz, 1H), 3.76-3.68 (m, 1H), 3.03 (t, J=6.1 Hz, 2H), 2.58 (t, J=6.1 Hz, 2H), 2.37-2.26 (m, 2H), 2.10 (quintet, J=6.1 2H), 1.90-1.76 (m, 4H), 1.75-1.66 (m, 2H). LRMS (ESI) m/z 473.8 [(M+H)⁺; calcd for C₂₃H₂₃N₂O₃S₂Cl: 473].

### Example 87

### N-(4-cyanophenyl)-3-[3-(cyclopentylamino)-4-oxo-4,5,6,7-tetrahydro-2-benzothiophen-1-yl]-1H-pyrazole-1-carboxamide

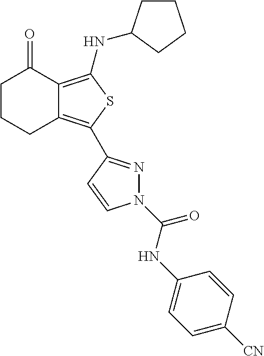 [SMILES: N#Cc1ccc(NC(=O)n2ccc(-c3sc(NC4CCCC4)c4c3CCCC4=O)n2)cc1]

The title compound was prepared by following the procedure outlined in Example 57, using 3-(cyclopentylamino)-1-(1H-pyrazol-3-yl)-6,7-dihydro-2-benzothiophen-4(5H)-one (Example 4) and 4-cyanophenyl isocyanate. NMR (400 MHz, CDCl₃) δ 9.20 (s, NH), 8.96 (d, J=7.1 Hz, NH), 8.28 (d, J=2.8 Hz, 1H), 7.79 (d, J=8.8 Hz, 2H), 7.69 (d, J=8.8 Hz, 2H), 6.60 (d, J=2.8 Hz, 1H), 3.81 (m, 1H), 2.94 (t, J=6.2 Hz, 2H), 2.49 (t, J=6.3 Hz, 2H), 2.02-2.16 (m, 4H), 1.69-1.82 (m, 6H) ppm. LRMS (ESI) m/z 446.1 [(M+H)⁺; calcd for C₂₄H₂₄N₅O₂S: 446].

### Example 88

### 6,6-Dimethyl-1-(5-methyl-1H-pyrazol-3-yl)-3-(methylsulfanyl)-6,7-dihydro-2-benzothiophen-4(5H)-one

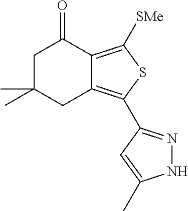 [SMILES: CSc1sc(-c2cc(C)[nH]n2)c2c1C(=O)CC(C)(C)C2]

### Step 1: 1-(But-2-ynoyl)-6,6-dimethyl-3-(methylsulfanyl)-6,7-dihydro-2-benzothiophen-4(5H)-one

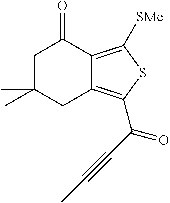 [SMILES: CC#CC(=O)c1sc(SC)c2c1CC(C)(C)CC2=O]

To a solution of 6,6-dimethyl-3-(methylsulfanyl)-4-oxo-4,5,6,7-tetrahydro-2-benzothiophene-1-carbaldehyde (100 mg, 0.39 mmol) [Prim, D.; Kirsch G. Synth. Commun. (1995), 25, 2449] in THF (2 mL) at −78° C. was added propynyllithiurn (22 mg, 0.43 mmol) in THF (2 mL). The reaction mixture was stirred, allowed to warm to RT over 1 h and then quenched with aqueous NH₄Cl. The mixture was extracted with dichloromethane and the organic layer was dried over Na₂SO₄, filtered and concentrated to give the title compound. LRMS (ESI) m/z 295.1 [(M+H)⁺; calcd for C₁₅H₁₉O₂S₂: 295].

### Step 2: 6,6-Dimethyl-1-(5-methyl-1H-pyrazol-3-yl)-3-(methylsulfanyl)-6,7-dihydro-2-benzothiophen-4(5H)-one

To a mixture of Dess-Martin periodinane (2.1 mL, 1.02 mmol, 15% in dichloromethane) was added pyridine (410 μL, 5.1 mmol) and the mixture was stirred for 5 min. A solution of the above product (100 mg, 0.34 mmol) in dichloromethane (1.1 mL) was then added and the mixture was stirred for 4 h. The reaction mixture was quenched with aqueous Na₂S₂O₃ and aqueous NaHCO₃, stirred 10 min and extracted with ether. The organic layer was dried over Na₂SO₄, filtered and concentrated and dissolved in ethanol (1 mL). Hydrazine (12 μL, 0.37 mmol) was then added and the mixture was heated to 80° C. and stirred for 4 h. The mixture was diluted with dichloromethane, washed with water and brine, dried over Na₂SO₄, filtered and concentrated. The title compound could be separated from uncyclized material via reverse-phase HPLC purification. ¹H NMR (400 MHz, CD₃OD) δ 6.27 (s, 1H), 2.83 (s, 2H), 2.63 (s, 3H), 2.39 (s, 2H), 2.35 (s, 3H) 2.03 (s, 6H) ppm. LRMS (ESI) m/z 307.2 [(M+H)⁺; calcd for C₁₅H₁₉N₂OS₂: 307].

By following the procedure outlined in Example 88, using the appropriate alkynyl lithium, the following compounds could be prepared (Examples 89-90).

| | | | LRMS |
|---|---|---|---|
| Ex. | Structure | Name | (M + H)⁺ |
| 89 | 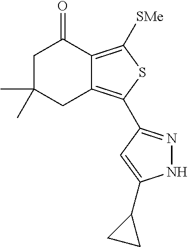 [SMILES: CSc1sc(-c2cc(C3CC3)[nH]n2)c2c1C(=O)CC(C)(C)C2] | 1-(5-Cyclopropyl- 1H-pyrazol-3-yl)- 6,6-dimethyl-3- (methylsulfanyl)- 6,7-dihydro-2- benzothiophen- 4(5H)-one | 333.1 |
| 90 | 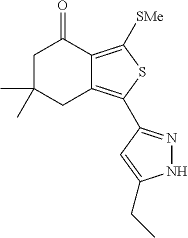 [SMILES: CCc1cc(-c2sc(SC)c3c2CC(C)(C)CC3=O)n[nH]1] | 1-(5-Ethyl-1H- pyrazol-3-yl)-6,6- dimethyl-3- (methylsulfanyl)- 6,7-dihydro-2- benzothiophen- 4(5H)-one | 321.1 |

### Example 91

### 5-Methyl-3-(methylsulfanyl)-1-(1H-pyrazol-3-yl)-6,7-dihydro-2-benzothiophen-4(5H)-one

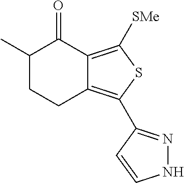 [SMILES: CSc1sc(-c2cc[nH]n2)c2c1C(=O)C(C)CC2]

### Step 1: 3-(Methylsulfanyl)-1-(1-{[2-(trimethylsilyl)ethoxy]methyl}-1H-pyrazol-3-yl)-6,7-dihydro-2-benzothiophen-4(5H)-one

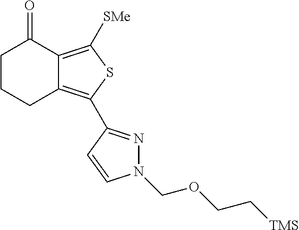 [SMILES: CSc1sc(-c2ccn(COCC[Si](C)(C)C)n2)c2c1C(=O)CCC2]

To a solution of 3-(methylsulfanyl)-1-(1H-pyrazol-3-yl)-6,7-dihydro-2-benzothiophen-4(5H)-one (Example 1) (2.64 g, 10.0 mmol) in THF (50 mL) at 0° C. was added sodium hydride (400 mg, 10.0 mmol, 60% dispersion). The reaction mixture was stirred for 30 min, [2-(chloromethoxy)ethyl](trimethyl)silane (1.67 g, 10.0 mmol) was added and the reaction mixture was stirred for 2 h. The reaction mixture was diluted with dichloromethane, washed with water and the organic layer was dried over Na₂SO₄, filtered and concentrated. The crude product was purified by silica gel chromatography (gradient elution, 0-5% methanol in dichloromethane) to provide the title compound as a mixture of N-1 protected and N-2 protected isomers. LRMS (ESI) m/z 395.0 [(M+H)⁺; calcd for C₁₈H₂₇N₂O₂S₂Si: 395].

### Step 2: 5-Methyl-3-(methylsulfanyl)-1-(1-{[2-(trimethylsilyl)ethoxy]methyl}-1H-pyrazol-3-yl)-6,7-dihydro-2-benzothiophen-4(5H)-one

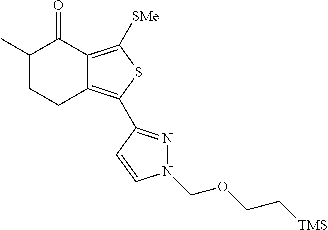 [SMILES: CSc1sc(-c2ccn(COCC[Si](C)(C)C)n2)c2c1C(=O)C(C)CC2]

To a solution of the above product (58 mg, 0.15 mmol) in THF (0.74 mL) at −78° C. was added lithium diisopropylamide (90 μL, 0.17 mmol, 1.8 M in THF)) dropwise, and the reaction mixture was stirred for 30 min. Methyl iodide (10 μL, 0.17 mmol) was added, the reaction mixture was warmed to RT, then heated to 50° C. and stirred for 45 h. The reaction mixture was diluted with dichloromethane, washed with water and the organic layer was dried over Na₂SO₄, filtered and concentrated. The crude product was purified by silica gel chromatography (gradient elution, 0-25% EtOAc in hexane) to provide the title compound as a mixture of N-1 protected and N-2 protected isomers. LRMS (ESI) m/z 409.0 [(M+H)⁺; calcd for C₁₉H₂₉N₂O₂S₂Si: 409].

### Step 3: 5-Methyl-3-(methylsulfanyl)-1-(1H-pyrazol-3-yl)-6,7-dihydro-2-benzothiophen-4(5H)-one

To a solution of the above product (16 mg, 0.04 mmol) in ethanol (1.9 mL) was added 3 N HCl (0.64 mL, 1.90 mmol). The reaction mixture was heated to 80° C. and stirred for 18 h. The reaction mixture was neutralized with 2.5 N NaOH and extracted four times with dichloromethane. The combined organics were dried over Na₂SO₄, filtered and concentrated to provide the title compound. ¹H NMR (400 MHz, CDCl₃) δ 7.64 (d, J=2.0 Hz, 1H), 6.49 (bs, 1H), 3.13-3.20 (dt, J=16.5, 4.5, 4.5 Hz, 1H), 2.87-2.95 (ddd, J=16.2, 11.4, 4.8 Hz, 1H), 2.62 (s, 3H), 2.53-2.59 (m, 1H), 2.13-2.19 (m, 1H), 1.79-1.89 (m, 1H), 1.26 (d, J=7.2 Hz, 3H) ppm. LRMS (ESI) m/z 279.1 [(M+H)⁺; calcd for C₁₃H₁₅N₂OS₂: 279].

### Example 92

### 3-(Methylsulfanyl)-5-propyl-1-(1H-pyrazol-3-yl)-6,7-dihydro-2-benzothiophen-4(5H)-one

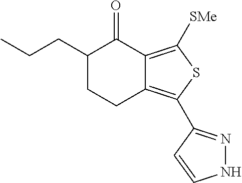 [SMILES: CCCC1CCc2c(-c3cc[nH]n3)sc(SC)c2C1=O]

The title compound was prepared according to the procedure given for Example 91 using 1-iodopropane. ¹H NMR (400 MHz, CDCl₃) δ 7.64 (d, J=2.3 Hz, 1H), 6.49 (bs, 1H), 3.11-3.16 (m, 1H), 2.87-2.94 (m, 1H), 2.61 (s, 3H), 2.42-2.47 (m, 1H), 2.13-2.21 (m, 1H), 1.83-1.95 (m, 2H), 1.37-1.57 (m, 6H), 0.95 (t, J=7.1 Hz, 3H) ppm. LRMS (ESI) m/z 307.1 [(M+H)⁺; calcd for C₁₅H₁₉N₂OS₂: 307].

### Assays

Activity of compounds against LRRK2 G2019S mutant kinase function was tested in a LANTHAScreen® assay as follows: Compounds were diluted in 100% DMSO to the desired test concentrations in white, low-volume 384-well plates (PerkinElmer ProxiPlate® PN: 6008280). TR-FRET kinase reactions (10 μL) were performed with 75 nM Fluorescein-ERM (LRRKtide, Invitrogen, PN:PV4901) substrate in a final assay buffer containing 100 μM ATP and 2 mM DTT diluted in commercially available Kinase Buffer S (Invitrogen PN: PV5213). The final solvent concentration in the assay was 1% DMSO. Reactions were initiated with the addition of 10 ng/well of LRRK2 02019S, GST-tagged protein (Invitrogen PN: PV4882). After incubation of the foil sealed reaction plates at room temperature with low speed mixing for 1 hour, the kinase reaction was stopped and the amount of substrate phosphorylation was detected by addition of 10 μL of 10 mM EDTA and 2 nM Tb-anti-pERM (pLRRKtide antibody, Invitrogen, PN: PV4898) antibody in TR-FRET dilution buffer (Invitrogen PN: PV3574). After a 30 minute incubation at room temperature with low speed mixing, foil seals were removed and measurements were obtained on a PerkinElmer EnVision with excitation 340 nm, emission 520 nm (channel 1): 495 nm (channel 2) settings. Generation of raw data and workup of data to derive the IC₅₀ (potency) and maximal inhibition at concentrations tested were performed using Merck's Assay Data Analyzer (ADA) software.

The IC₅₀ (potency) was evaluated for the example compounds presented above. IC₅₀ values determined in accordance with the above-described assay are reported in the following lists:

Examples 1 to 25 (IC₅₀ value in nM):
Example 1 (190); Example 2 (3500); Example 3 (430); Example 4 (160); Example 5 (230); Example 6 (2800); Example 7 (93); Example 8 (180); Example 9 (140); Example 10 (240); Example 11 (200); Example 12 (290); Example 13 (520); Example 14 (860); Example 15 (450); Example 16 (220); Example 17 (450); Example 18 (220); Example 19 (60); Example 20 (490); Example 21 (200); Example 22 (130); Example 23 (180); Example 24 (70); Example 25 (740).

Examples 26 to 50 (IC₅₀ Value in nM):
Example 26 (120); Example 27 (140); Example 28 (160); Example 29 (250); Example 30 (180); Example 31 (1900); Example 32 (4000); Example 33 (120); Example 34 (1300); Example 35 (150); Example 36 (760); Example 37 (180); Example 38 (290); Example 39 (240); Example 40 (580); Example 41 (2000); Example 42 (420); Example 43 (4000); Example 44 (380); Example 45 (250); Example 46 (210); Example 47 (290); Example 48 (620); Example 49 (97); Example 50 (180).

Examples 51 to 75 (IC₅₀ Value in nM):
Example 51 (220); Example 52 (300); Example 53 (430); Example 54 (790); Example 55 (4200); Example 56 (2200); Example 57 (1400); Example 58 (110); Example 59 (93); Example 60 (520); Example 61 (110); Example 62 (86); Example 63 (110); Example 64 (85); Example 65 (330); Example 66 (110); Example 67 (330); Example 68 (130); Example 69 (4200); Example 70 (1300); Example 71 (1700); Example 72 (1700); Example 73 (280); Example 74 (190); Example 75 (720).

Examples 76 to 92 (IC₅₀ Value in nM):
Example 76 (980); Example 77 (260); Example 78 (1500); Example 79 (600); Example 80 (570); Example 81 (970); Example 82 (3400); Example 83 (4000); Example 84 (400); Example 85 (1100); Example 86 (66); Example 87 (330); Example 88 (1400); Example 89 (470); Example 90 (450); Example 91 (150); Example 92 (310)

Kinome selectivity profiling was performed using two example compounds according to the manufacturer's instructions using Caliper LifeSciences' ProfilerPro Kinase Selectivity Assay Kits. Compounds prepared in Examples 1 and 7 were tested at 10 uM and data were recorded using the Caliper LabChip® EZ Reader™. Each compounds' percent inhibition of the kinase activity measured in this manner is reported in the following lists.

Compound of Example 1, Kinome Selectivity:
ABL, 12.7; ABL H396P, 10.3; ABL Q252H, 16.9; ABL T315I, 9.9; ABL1 E255K, 2.8; ABL1 G250E, 5.0; ABL1 Y253F, 2.5, AKT1, −3.2; AKT2, −6.8; AKT3, −2.2; ALK, 23.8, AMPKA1, 16.4; AMPKA2/B1/G1, −2.7; ARG −6.0; AURA, 44.0; AURB, 57.7; AURC, 27.9; AXL, 13.5; BLK, 6.6; BMX, −10.9; BRSK1, −0.2; BRSK2, −1.7; BTK, 3.1; CAMK1A, 7.4; CAMK1D, −3.1; CAMK2A, −4.8; CAMK2B, 6.3; CAMK2G, 3.1; CAMK4, 20.1; CASEIN K1G2, 6.9; CDK1/CYCB1, 5.3; CDK2, 18.4; CDK3, 21.4; CDK5/P25, 9.5; CHK1, 0.0; CHK2, 48.7; CK1D, 44.6; CK1E, 10.9; CK1G1, 6.6; CK1G3, 1.1; CLK2, 31.3; CSNK1A1, 12.4; C-TAK1, 5.3; DAPK1, 7.2; DCAMKL1, −2.5; DCAMKL2, −0.8; DDR2, 0.8; DYRK1A, 44.0; DYRK1B, 40.2; DYRK3, 11.7; DYRK4, −1.1; EGFR, −3.1; EGFR T790M, 1.8; EGFR T790M L858R, 3.9; EPHA1, 2.8; EPHA2, 0.7; EPHA3, −2.2; EPHA4, −0.8; EPHA5, −3.1; EPHA8, −0.2; EPHB1, 3.0; EPHB2, 2.6; EPHB3, −1.0; EPHB4, 0.6; ERK1, −2.0; ERK2, 4.5; FER, 11.6; FES, −2.8; FGFR1, 1.9; FGFR1 V561M, 4.4; FGFR2, 4.9; FGFR2 N549H, 1.4; FGFR3, 9.0; FGFR3 K₆₅₀E, 3.7; FGFR4, 5.9; FGR, −7.2; FLT1, 1.2; FLT3, 11.4; FLT-3 D835Y, 39.3; FLT4, 22.5; FMS, −36.1; FYN, 1.2; GCK, 6.3; GSK3A, 8.5; GSK3B, 5.4; HCK, 12.5; HER4, −2.4; HGK, 13.8; HIPK1, 15.9; HIPK2, 25.2; IGF1R, 5.4; IKKB, 9.3; IKKE, −1.5; INSR, 3.4; IRAK4, 31.1; ITK, 4.8; JAK2, 19.4; KDR, 16.4; KIT, 19.2; KIT T6701, 10.6; LCK, 4.0; LOK, 7.5; LRRK2, 93.9; LRRK2 (G2019S), 88.3; LTK, 16.0; LYN, 10.2; LYNB, 8.2; MAPKAPK2, 4.1; MAPKAPK3, 0.1; MAPKAPK5, 4.4; MARK1, −2.9; MARK2, 0.3; MARK4, −9.4; MELK, 11.0; MER, 23.8; MET, 8.2; MET M1250T, 5.1; MINK, 24.8; MNK1, 1.7; MSK1, 5.1; MSK2, 4.2; MST1, 7.2; MST1R, −1.3; MST2, 12.3; MST3, −2.2; NEK1, 9.1; NEK2, −0.8; NTRK2, 16.9; NUAK1, 44.2; P38A, −2.3; P38A/SAPK2A T106M, 1.2; P38B2, −1.3; P38D, 4.2; P38G, 23.3; P70S6K, 5.7; PAK2, 4.6; PAK3, −13.1; PAK4, 6.1; PAK5, −0.6; PASK, 2.6; PDGFR A(550), 1.5; PDGFRA, 15.7; PDGFRA D842V, 33.9; PDGFRB, 22.1; PHKG1, 6.3; PHKG2, −5.5; PIM1, 26.3; PIM2, 1.6; PIM-3, 23.7; PKACA, −6.0; PKCI, −14.0; PKD1, 5.5; PKD2, −2.5; PKD3, 12.0; PKG1A, −5.9; PKG1B, 10.8; PRKCA, 2.6; PRKCB1, −5.1; PRKCB2, −7.4; PRKCD, −14.8; PRKCE, −2.7; PRKCG, 10.0; PRKCH, 1.1; PRKCQ, −0.4; PRKCZ, 0.4; PRKX, −0.6; PYK2, 4.2; RAF-1, 3.1; RET, 24.4; RET V804L, 41.5; RET Y791F, 37.6; ROCK1, −3.1; ROCK2, −3.5; ROS, 33.7; RSK1, 9.5; RSK2, 5.5; RSK3, 9.4; RSK4, 2.9; SGK, −5.1; SGK2, 10.6; SGK3, 4.6; SRC, 7.3; SRM, 3.5; SYK, 49.9; TEC, 0.6; TRKC, 10.3; TSSK1, 24.0; TSSK2, 20.1; TXK, 22.7; TYRO3, 8.6; YES, −4.4; ZIPK, 8.1;

Compound of Example 7, Kinome selectivity:
ABL, 1.9; ABL H396P, 3.1; ABL Q252H, 3.9; ABL T315I, 2.6; ABL1 E255K, 7.5; ABL1 G250E, 6.4; ABL1 Y253F, 10.6; AKT1, 0.1; AKT2, −6.8; AKT3, −2.7; ALK, 44.7; AMPKA1, −0.5; AMPKA2/B1/G1, 4.5; ARG, 8.6; AURA, 11.0; AURB, −3.1; AURC, 3.5; AXL, 12.3; BLK, 3.2; BMX, −30.7; BRSK1, 3.8; BRSK2, −11.1; BTK, 4.8; CAMK1A, 6.0; CAMK1D, 4.0; CAMK2A, 1.7; CAMK2B, 6.0; CAMK2G, 4.4; CAMK4, −1.5; CASEIN K1G2, 8.6; CDK1/CYCB1, −6.7; CDK2, 2.1; CDK3, 2.5; CDK5/P25, −11.7; CHK1, 8.0; CHK2, 3.7; CK1D, 12.9; CK1E, 5.1; CK1G1, 10.9; CK1G3, 7.7; CLK2, 1.3; CSNK1A1, 2.0; C-TAK1, 4.7; DAPK1, −2.0; DCAMKL1, −2.1; DCAMKL2, −4.2; DDR2, −3.9; DYRK1A, 0.0; DYRK1B, 4.7; DYRK3, −8.5; DYRK4, 0.4; EGFR, 2.2; EGFR T790M, −1.9; EGFR T790M L858R, 13.7; EPHA1, −0.2; EPHA2, 0.7; EPHA3, −2.9; EPHA4, −3.5; EPHA5, 1.5; EPHA8, −3.8; EPHB1, 3.3; EPHB2, 2.3; EPHB3, 2.2; EPHB4, 0.1; ERK1, 1.9; ERK2, −0.9; FER, −0.2; FES, −3.1; FGFR1, 7.4; FGFR1 V561M, 7.9; FGFR2, 2.6; FGFR2 N549H, −5.8; FGFR3, −2.6; FGFR3 K650E, −4.2; FGFR4, −0.1; FGR, −1.6; FLT1, 11.8; FLT3, 4.6; FLT-3 D835Y, 2.7; FLT4, 2.0; EMS, −6.5; FYN, 2.5; GCK, 11.8; GSK3A, −8.3; GSK3B, 0.5; HCK, −1.1; HER4, −2.2; HGK, 3.9; HIPK1, −1.0; HIPK2, 5.0; IGF1R, −3.7; IKKB, 0.5; IKKE, 1.3; INSR, 0.9; IRAK4, 3.7; ITK, −2.9; JAK2, 5.3; KDR, −0.8; KIT, −7.3; KIT T670I, 3.0; LCK, 23.3; LOK, −32.6; LRRK2, 83.1; LRRK2 (G2019S), 78.3; LTK, 19.5; LYN, 4.0; LYNB, −4.9; MAPKAPK2, 2.0; MAPKAPK3, −1.0; MAPKAPK5, 1.9; MARK1, −0.6; MARK2, 9.2; MARK4, 11.6; MELK, 7.3; MER, 8.0; MET, −0.9; MET M1250T, −0.3; MINK, −1.5; MNK1, −5.6; MSK1, −2.8; MSK2, 10.4; MST1, −3.0; MST1R, −7.0; MST2, −2.3; MST3, −10.9; NEK1, 1.4; NEK2, 0.9; NTRK2, 7.0; NUAK1, −4.2; P38A, 0.6; P38A/SAPK2A T106M, 3.7; P38B2, −0.7; P38D, −0.3; P38G, −0.2; P70S6K, −4.8; PAK2, 2.4; PAK3, −170.0; PAK4, −8.9; PAK5, −4.4; PASK, -10.8; PDGFR A(550), −13.8; PDGFRA, −0.7; PDGFRA D842V, 3.7; PDGFRB, −1.4; PHKG1, 4.6; PHKG2, −2.0; PIM1, −16.3; PIM2, −1.6; PIM-3, 8.2; PKACA, −5.4; PKCI, −8.9; PKD1, −10.6; PKD2, −4.2; PKD3, 2.6; PKG1A, 1.9; PKG113, −0.6; PRKCA, 4.3; PRKCB1, −3.9; PRKCB2, 6.4; PRKCD, 12.6; PRKCE, 3.9; PRKCG, 2.3; PRKCH, 0.8; PRKCQ, −4.7; PRKCZ, 0.5; PRKX, −6.6; PYK2, −0.6; RAF-1, −0.2; RET, 20.7; RET V804L, 15.7; RET Y791F, 11.1; ROCK1, 5.1; ROCK2, 0.9; ROS, 42.7; RSK1, 3.3; RSK2, 3.5; RSK3, 2.0; RSK4, −5.6; SGK, −1.3; SGK2, 5.4; SGK3, 4.6; SRC, 1.5; SRM, 3.0; SYK, 5.8; TEC, −3.2; TRKC, 0.5; TSSK1, 8.3; TSSK2, −2.3; TXK, 8.4; TYRO3, 10.5; YES, −11.1; ZIPK, 1.0;

It can be seen from the foregoing data that the compounds of Formula I show unexpected potency and selectivity for inhibition of LRRK2 kinase activity. While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A pharmaceutical composition comprising a LRRK2 kinase activity-inhibiting compound of the structure of Formula I: 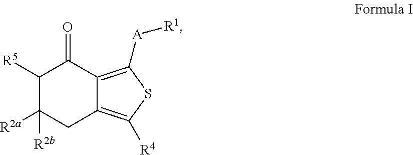
or comprising a pharmaceutically acceptable salt of a compound of Formula I, wherein:
“A” is —CH₂— and —R¹ is —C₁₋₁₀-alkyl; or
“A” is: —S—; —SO—; —SO₂—; —O—; or —NR^{a}—, wherein —R^{a} is —H, —C₁₋₂₀-alkyl, or —R^{a} is taken together with —R¹ to form a cyclo-amino moiety of the formula: 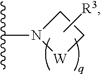
wherein “q” is an integer of 1 to 4, “W” is —CH₂— or —(N—C₁₋₂₀-alkyl)-, —R³ is one or more moieties which are independently: —OH; —C₁₋₆-alkyl; —C₁₋₆-alkoxy,
and wherein, when “A” is not (—CH₂—), —R¹ is:
(a) —C₁₋₈-alkyl, optionally substituted independently for each occurrence with:
(i) —(N═N⁺═N⁻); (ii) -halogen; (iii) —C₁₋₈-alkoxy, optionally substituted; (iv) —OH; (v) morpholinyl-; (vi) pyridyl-; (vii) moieties of the formula (—N(R^{b})₂), where —R^{b} is independently —H or —C₁₋₁₀-alkyl; (viii) furanyl-; (ix) -aryl, optionally substituted, independently for each occurrence, with: (1) —NR^{c}—(C═O)—CH₃, where —R^{c} is —H or —C₁₋₆-alkyl; (2) -alkoxy; (3) halogen-; (x) -aryloxy; (xi) a moiety of Formula A 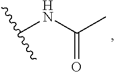 [SMILES: *NC(C)=O]
or Formula B 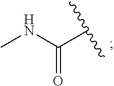 [SMILES: *C(=O)NC]
(xii) a moiety of the formula 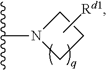
wherein, “q” is an integer of 1 to 4, and wherein independently, for each occurrence, a ring carbon is optionally substituted with —R^{d1}, wherein —R^{d1} is —C₁₋₆-alkyl; (xiii) a moiety of the formula —CH═CHR^{d2}, wherein —R^{d2} is —H or —C₁₋₆-alkyl; (xiv) —Si(R^{e})₃, wherein —R^{e} is a —C₁₋₄-alkyl which is optionally substituted with -fluorine;
(b) thiopenyl-; (c) heteroaryl-; (d) pyridyl-; (e) furanyl-;
(f) -aryl, optionally substituted, independently for each occurrence with: (i) —N(R^{f})—(C═O)—CH₃, wherein —R^{f} is —H or —C₁₋₆-alkyl; (ii) —C₁₋₂₀-alkoxy; or (iii) halogen-;
(g) a moiety of the formula: 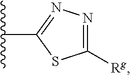
wherein —R^{g} is —C₁₋₆-alkyl or —S—C₁₋₆-alkyl;
—R^{2a} and a R^{2b} are independently —C₁₋₆-alkyl or —H;
—R⁴ is a substituent of the Formula: 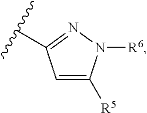
wherein: —R⁶ is:
(a) —H;
(b) —C₁₋₆-alkyl;
(c) —SO₂—C₁₋₆-alkyl;
(d) —(C═O)—NH—R^{h}, wherein —R^{h} is:
(I) —(CH₂)₀₋₄—C₆₋₁₀-aryl, the -aryl moiety optionally comprising up to three substituents which are independently for each occurrence: (i) -halogen; (ii) —O—C₁₋₆-alkyl; (iii) —S—C₁₋₆-alkyl or (iv) —CN;
(II) pyridinyl-, optionally substituted, independently for each occurrence, with -halogen;
(III) piperidinyl-, which is bonded to the substrate through a carbon atom of the ring, and is optionally N-substituted with an acyl-moiety; or
(IV) —C₁₋₈-alkyl;
(e) —(C═O)—CH₂—Rⁱ, wherein —Rⁱ is: (i) —H; (ii) p-chlorobenzyl-; (iii) —C₁₋₆-alkyl; or (iv) —C₁₋₂₀-alkoxy; or
(f) —(C═O)—R^{k}, wherein —R^{k} is: (i) —C₁₋₆-alkyl; (ii) phenyl-, optionally substituted with up to 3 substituents which are independently for each occurrence: cyano-; halogen-; or —C₁₋₆-alkoxy (—O—C₁₋₆-alkyl); or (iii) —(NH)—C₁₋₆-linear-alkyl-aryl; and
—R⁵ is: (a) —H; (h) —C₁₋₆-alkyl; or (c)-(CH₂)₀₋₄—C₆₋₁₀-aryl, wherein the -aryl moiety is optionally substituted by up to three substituents which are independently for each occurrence: (i) -halogen; (ii) —O—C₁₋₆-alkyl; (iii) —S—C₁₋₆-alkyl or (iv) —CN.

2. A LRRK2 kinase activity-inhibiting compound of the structure of Formula I: 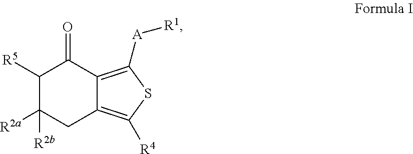
or a pharmaceutically acceptable salt thereof, wherein:
“A” is —CH₂— and —R¹ is —C₁₋₁₀-alkyl; or
“A” is: —S—; —SO—; —SO₂—; —O—; or wherein —R^{a} is —H, —C₁₋₂₀-alkyl, or —R^{a} is taken together with —R¹ to form a cyclo-amino moiety of the formula: 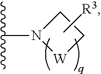
wherein “q” is an integer of 1 to 4, “W” is —CH₂— or —(N—C₁₋₂₀-alkyl)-, —R³ is one or more moieties which are independently: —OH; —C₁₋₆-alkoxy,
and wherein, when “A” is not (—CH₂—), —R′ is:
(a) —C₁₋₈-alkyl, optionally substituted independently for each occurrence with:
(i) —(N═N⁺═N⁻); (ii) -halogen; (iii) —C₁₋₈-alkoxy, optionally substituted; (iv) —OH; (v) morpholinyl-; (vi) pyridyl-; (vii) moieties of the formula (—N(R^{b})₂), where —R^{b} is independently —H or —C₁₋₁₀-alkyl; (viii) furanyl-; (ix) -aryl, optionally substituted, independently for each occurrence, with: (1) —NR^{c}—(C═O)—CH₃, where —R^{c} is —H or —C₁₋₆-alkyl; (2) -alkoxy; (3) halogen-; (x) -aryloxy; (xi) a moiety of Formula A 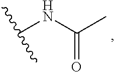 [SMILES: *NC(C)=O]
or Formula B 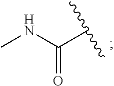 [SMILES: *C(=O)NC]
(xii) a moiety of the formula 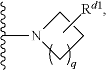
wherein, “q” is an integer of 1 to 4, and wherein independently, for each occurrence, a ring carbon is optionally substituted with —R^{d1}, wherein —R^{d1} is —C₁₋₆-alkyl; (xiii) a moiety of the formula —CH═CHR^{d2}, wherein —R^{d2} is —H or —C₁₋₆-alkyl; (xiv) —Si(R^{e})₃, wherein —R^{e} is a —C₁₋₄-alkyl which is optionally substituted with -fluorine;
(b) thiopenyl-;
(e) heteroaryl-;
(d) pyridyl-;
(e) furanyl-;
(f) -aryl, optionally substituted, independently for each occurrence with: (i) —N(R^{f})—(C═O)—CH₃, wherein —R^{f} is —H or —C₁₋₆-alkyl; (ii) —C₁₋₂₀-alkoxy; or (iii) halogen-;
(g) a moiety of the formula: 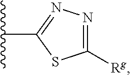
wherein —R^{g} is —C₁₋₆-alkyl or —S—C₁₋₆-alkyl;
—R^{2a} and —R^{2b} are independently —C₁₋₆-alkyl or —H;
—R⁴ is a substituent of the Formula: 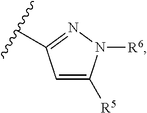
wherein: —R⁶ is:
(a) —H;
(b) —C₁₋₆-alkyl;
(c) —SO₂—C₁₋₆-alkyl;
(d) —(C═O)—NH—R^{h}, wherein —R^{h} is:
(I) —(CH₂)₀₋₄—C₆₋₁₀-aryl, the -aryl moiety optionally comprising up to three substituents which are independently for each occurrence: (i) -halogen; (ii) —O—C₁₋₆-alkyl; (iii) —S—C₁₋₆-alkyl or (iv) —CN;
(II) pyridinyl-, optionally substituted, independently for each occurrence, with -halogen;
(III) piperidinyl-, which is bonded to the substrate through a carbon atom of the ring, and is optionally N-substituted with an acyl-moiety; or
(IV) —C₁₋₈-alkyl;
(e) —(C═O)—CH₂—Rⁱ, wherein —Rⁱ is: (i) —H; (ii) p-chlorobenzyl-; (iii) —C₁₋₆-alkyl; or (iv) —C₁₋₂₀-alkoxy; or
(f) —(C═O)—R^{k}, wherein —R^{k} is: (i) (ii) phenyl-, optionally substituted with up to 3 substituents which are independently for each occurrence: cyano-; halogen-; or —C₁₋₆-alkoxy (—O—C₁₋₆-alkyl); or (iii) —(NH)—C₁₋₆-linear-alkyl-aryl; and
—R⁵ is: (a) —H; (b) —C₁₋₆-alkyl; or (c)-(CH₂)₀₋₄—C₆₋₁₀-aryl, wherein the -aryl moiety is optionally substituted by up to three substituents which are independently for each occurrence: (i) -halogen; (ii) —O—C₁₋₆-alkyl; (iii) —S—C₁₋₆-alkyl or (iv) —CN
with the proviso that:
when “A” is —S—, substituents —R⁵ and —R⁶ on the —R⁴ moiety are both —H, and —R^{2a} and —R^{2b} are both -methyl, —R¹ is not the moiety [R^{x1}—CH₂—], wherein —R^{x1} is: —CF₃; —CH₂-phenyl; -phenyl; —H; a moiety of the Formula of Cmpd “X”: 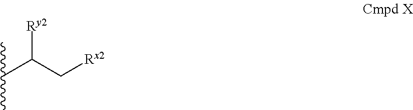
wherein, —R^{x2} and —R^{y2} are selected to give a moiety of Cmpd “X” defining M-1 to M-5, as shown in Table I:
TABLE I

| | Cmpd X | | | | |
|---|---|---|---|---|---|
| | M-1 | M-2 | M-3 | M-4 | M-5 |
| —R^{x2} | —H | —OH | —OCH₃ | —H | -Phenyl |
| —R^{y2} | —H | —H | —H | —CH₃ | —H |

or a moiety of Cmpd “Y”: 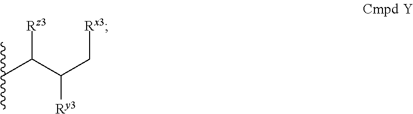
wherein —R^{x3}, —R^{y3}, and —R^{z3} are selected to give a moiety of Cmpd “Y” defining M-1 to M-7, as shown in Table II:
TABLE II

| Cmpd Y | M-1 | M-2 | M-3 | M-4 | M-5 | M-6 | M-7 |
|---|---|---|---|---|---|---|---|
| —R^{x3} | —H | —OH | —H | —OH | —H | —CH₃ | —CH₂—CH₃ |
| —R^{y3} | —H | —H | —OH | —OH | —CH₃ | —H | —H |
| —R^{z3} | —H | —H | —H | —H | —H | —H | —H; |

or, when “A” is —N—, —R⁵ and —R⁶ on the —R⁴ substituent are both —H, and —R^{2a} and —R² are both -methyl, —R¹ is not a —C₁₋₄ alkyl moiety; or when “A” is —SO₂— and —R^{2a} and —R^{2b} are both -methyl, —R¹ is not -methyl.

3. The formulation of claim 1 wherein, in the compound of Formula I, —R^{2a} and —R^{2b} are -methyl, “A-R¹” is —S—R^{1a}, wherein —R^{1a} is a moiety selected from the definition of —R^{1a} appearing in Table III, —R⁵ is —H, and —R⁴ is a moiety of Formula IIA:

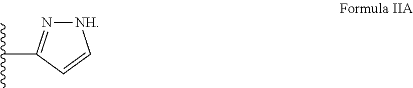 [SMILES: *c1cc[nH]n1]

4. The formulation of claim 1 wherein, in the compound of Formula I, “A-R¹” is —N—R^{1a}, —R⁵ is —H, —R⁴ is a moiety of Formula IIC: 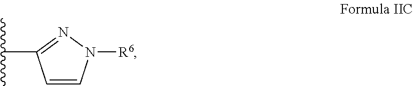
and wherein —R^{1a}, —R^{2a}, —R^{2b}, and —R⁶ are selected from their respective definitions appearing in Table IV.

5. The formulation of claim 1 wherein, in the compound of Formula I, —R⁵ is —H, “A” is —O—, —R¹″ is —R^{1a}, wherein —R^{1a}, along with —R^{2a} and —R^{2b} are selected from there respective definitions appearing in Table VI, and wherein —R⁴ is a moiety of Formula IIA:

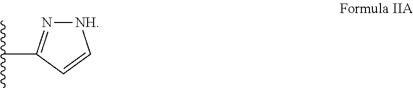 [SMILES: *c1cc[nH]n1]

6. The formulation of claim 1 wherein, in the compound of Formula I, “A-R¹” is —S-cyclopentane, —R⁵ is —H, —R⁴ is a moiety of Formula IIA: 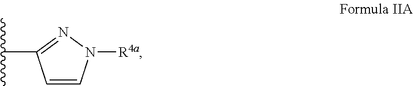
and wherein —R^{4a}, —R^{2a} and —R^{2b} are selected from there respective definitions appearing in Table VII.

7. The formulation of claim 1 wherein, in the compound of Formula I, “A-R¹” is —S—CH₃, —R⁵ is —H, —R⁴ is a moiety of Formula IIA: 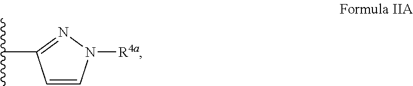
and wherein —R^{4a}, —R^{2a} and —R^{2b} are selected from there respective definitions appearing in Table VIII.

8. The formulation of claim 1 wherein, in the compound of Formula I, “A-R¹” is —S—CH₃, —R⁵ is —H, —R⁴ is a moiety of Formula IIB: 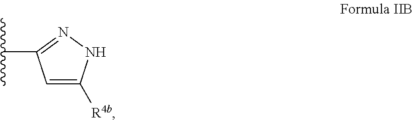
and wherein —R^{4b}, —R^{2a} and —R^{2b} are selected from there respective definitions appearing in Table IX.

9. The formulation of claim 1 wherein, in the compound of Formula I is a compound of the Formulae IB-28, IB-29, IB-30, IB31, IC-01, IC-02, ID-01, ID-02, ID-03, IE-01, IE-02, IG, IJ-01 or IJ-02.

10. A formulation comprising at least one of the following compounds:
3-(Cyclopentylsulfanyl)-6,6-dimethyl-1-(1H-pyrazol-3-yl)-6,7-dihydro-2-benzothiophen-4(5H)-one;
3-[(6-Hydroxyhexyl)sulfanyl]-6,6-dimethyl-1-(1H-pyrazol-3-yl)-6,7-dihydro-2-benzothiophen-4(5H)-one;
3-[(6-Aminohexyl)sulfanyl]-6,6-dimethyl-1-(1H-pyrazol-3-yl)-6,7-dihydro-2-benzothiophen-4(5H)-one;
3-(Cyclopentylamino)-6,6-dimethyl-1-(1H-pyrazol-3-yl)-6,7-dihydro-2-benzothiophen-4(5H)-one;
3-[3-(Cyclopentylsulfanyl)-6,6-dimethyl-4-oxo-4,5,6,7-tetrahydro-2-benzothiophen-1-yl]-N-[4-(trifluoromethyl)phenyl]-1H-pyrazole-1-carboxamide;
N-(4-Chlorophenyl)-3-[3-(cyclopentylsulfanyl)-6,6-dimethyl-4-oxo-4,5,6,7-tetrahydro-2-benzothiophen-1-yl]-1H-pyrazole-1-carboxamide;
N-(4-Cyanophenyl)-3-[3-(cyclopentylsulfanyl)-6,6-dimethyl-4-oxo-4,5,6,7-tetrahydro-2-benzothiophen-1-yl]-1H-pyrazole-1-carboxamide; or
N-(4-chlorophenyl)-3-[3-(cyclopentylsulfanyl)-4-oxo-4,5,6,7-tetrahydro-2-benzothiophen-1-yl]-1H-pyrazole-1-carboxamide.

11. A formulation comprising at least one of the following compounds or a salt thereof:
3-(Methylsulfanyl)-1-(1H-pyrazol-3-yl)-6,7-dihydro-2-benzothiophen-4(5H)-one;
3-(Cyclopentylsulfanyl)-1-(1H-pyrazol-3-yl)-6,7-dihydro-2-benzothiophen-4(5H)-one;
3-(Cyclopentylamino)-1-(1H-pyrazol-3-yl)-6,7-dihydro-2-benzothiophen-4(5H)-one;
6,6-Dimethyl-3-(methylsulfanyl)-1-(1H-pyrazol-3-yl)-6,7-dihydro-2-benzothiophen-4(5H)-one;
3-(Ethylsulfanyl)-6,6-dimethyl-1-(1H-pyrazol-3-yl)-6,7-dihydro-2-benzothiophen-4(5H)-one;
6,6-Dimethyl-3-(propan-2-ylsulfanyl)-1-(1H-pyrazol-3-yl)-6,7-dihydro-2-benzothiophen-4(5H)-one;
3-[(2-Hydroxyethyl)sulfanyl]-6,6-dimethyl-1-(1H-pyrazol-3-yl)-6,7-dihydro-2-benzothiophen-4(5H)-one;
3-[(3-Hydroxypropyl)sulfanyl]-6,6-dimethyl-1-(1H-pyrazol-3-yl)-6,7-dihydro-2-benzothiophen-4(5H)-one;
3-[(2-Hydroxypropyl)sulfanyl]-6,6-dimethyl-1-(1H-pyrazol-3-yl)-6,7-dihydro-2-benzothiophen-4(5H)-one;
6,6-Dimethyl-3-[(2-methylbutyl)sulfanyl]-1-(1H-pyrazol-3-yl)-6,7-dihydro-2-benzothiophen-4(5H)-one;
3-[(3-Azidopropyl)sulfanyl]-6,6-dimethyl-1-(1H-pyrazol-3-yl)-6,7-dihydro-2-benzothiophen-4(5H)-one;
3-[(Cyclopropylmethyl)sulfanyl]-6,6-dimethyl-1-(1H-pyrazol-3-yl)-6,7-dihydro-2-benzothiophen-4(5H)-one;
N-(6-{[5,5-Dimethyl-7-oxo-3-(1H-pyrazol-3-yl)-4,5,6,7-tetrahydro-2-benzothiophen-1-yl]sulfanyl}hexyl)acetamide;
6,6-Dimethyl-3-[(2-methylpropyl)sulfanyl]-1-(1H-pyrazol-3-yl)-6,7-dihydro-2-benzothiophen-4(5H)-one;
3-(Butylsulfanyl)-6,6-dimethyl-1-(1H-pyrazol-3-yl)-6,7-dihydro-2-benzothiophen-4(5H)-one;
6,6-Dimethyl-3-(propylsulfanyl)-1-(1H-pyrazol-3-yl)-6,7-dihydro-2-benzothiophen-4(5H)-one;
6,6-Dimethyl-1-(1H-pyrazol-3-yl)-3-[(2,2,2-trifluoroethyl)sulfanyl]-6,7-dihydro-2-benzothiophen-4(5H)-one;
3-(Butan-2-ylsulfanyl)-6,6-dimethyl-1-(1H-pyrazol-3-yl)-6,7-dihydro-2-benzothiophen-4(5H)-one;
3-[(3-Chloropropyl)sulfanyl]-6,6-dimethyl-1-(1H-pyrazol-3-yl)-6,7-dihydro-2-benzothiophen-4(5H)-one;
6,6-Dimethyl-3-(propylamino)-1-(1H-pyrazol-3-yl)-6,7-dihydro-2-benzothiophen-4(5H)-one;
6,6-Dimethyl-3-(propan-2-ylamino)-1-(1H-pyrazol-3-yl)-6,7-dihydro-2-benzothiophen-4(5H)-one;
3-(Ethylamino)-6,6-dimethyl-1-(1H-pyrazol-3-yl)-6,7-dihydro-2-benzothiophen-4(5H)-one;
3-(Cyclohexylamino)-6,6-dimethyl-1-(1H-pyrazol-3-yl)-6,7-dihydro-2-benzothiophen-4(5H)-one;
3-(Cyclopropylamino)-6,6-dimethyl-1-(1H-pyrazol-3-yl)-6,7-dihydro-2-benzothiophen-4(5H)-one;
3-[(6-Hydroxyhexyl)amino]-6,6-dimethyl-1-(1H-pyrazol-3-yl)-6,7-dihydro-2-benzothiophen-4(5H)-one;
3-[(trans-4-Hydroxycyclohexyl)amino]-6,6-dimethyl-1-(1H-pyrazol-3-yl)-6,7-dihydro-2-benzothiophen-4(5H)-one;
3-[3-(Cyclopentylsulfanyl)-6,6-dimethyl-4-oxo-4,5,6,7-tetrahydro-2-benzothiophen-1-yl]-N-(4-methoxyphenyl)-1H-pyrazole-1-carboxamide;
3-[3-(Cyclopentylsulfanyl)-6,6-dimethyl-4-oxo-4,5,6,7-tetrahydro-2-benzothiophen-1-yl]-N-[3-(methylsulfanyl)phenyl]-1H-pyrazole-1-carboxamide;
3-[3-(Cyclopentylsulfanyl)-6,6-dimethyl-4-oxo-4,5,6,7-tetrahydro-2-benzothiophen-1-yl]-N-(naphthalen-2-yl)-1H-pyrazole-1-carboxamide;
3-[3-(Cyclopentylsulfanyl)-6,6-dimethyl-4-oxo-4,5,6,7-tetrahydro-2-benzothiophen-1-yl]-N-(2-methoxyphenyl)-1H-pyrazole-1-carboxamide;
N-(3-Cyanophenyl)-3-[3-(cyclopentylsulfanyl)-6,6-dimethyl-4-oxo-4,5,6,7-tetrahydro-2-benzothiophen-1-yl]-1H-pyrazole-1-carboxamide;
N-(4-Cyanophenyl)-3-[3-(cyclopentylamino)-6,6-dimethyl-4-oxo-4,5,6,7-tetrahydro-2-benzothiophen-1-yl]-1H-pyrazole-1-carboxamide;
5-Methyl-3-(methylsulfanyl)-1-(1H-pyrazol-3-yl)-6,7-dihydro-2-benzothiophen-4(5H)-one; or
3-(Methylsulfanyl)-5-propyl-1-(1H-pyrazol-3-yl)-6,7-dihydro-2-benzothiophen-4(5H)-one.

12. A formulation comprising at least one of the following compounds or a salt thereof:
3-[(2-Methoxyethyl)sulfanyl]-6,6-dimethyl-1-(1H-pyrazol-3-yl)-6,7-dihydro-2-benzothiophen-4(5H)-one;
3-[(Furan-2-ylmethyl)sulfanyl]-6,6-dimethyl-1-(1H-pyrazol-3-yl)-6,7-dihydro-2-benzothiophen-4(5H)-one;
3-[(6-Azidohexyl)sulfanyl]-6,6-dimethyl-1-(1H-pyrazol-3-yl)-6,7-dihydro-2-benzothiophen-4(5H)-one;
6,6-Dimethyl-3-{[3-(4-methylpiperazin-1-yl)propyl]amino}-1-(1H-pyrazol-3-yl)-6,7-dihydro-2-benzothiophen-4(5H)-one;
3-[(Furan-2-ylmethyl)amino]-6,6-dimethyl-1-(1H-pyrazol-3-yl)-6,7-dihydro-2-benzothiophen-4(5H)-one;
6,6-Dimethyl-3-{[2-(morpholin-4-yl)ethyl]amino}-1-(1H-pyrazol-3-yl)-6,7-dihydro-2-benzothiophen-4(5H)-one;
6,6-Dimethyl-1-(1H-pyrazol-3-yl)-3-{[2-(pyridin-2-yl)ethyl]amino}-6,7-dihydro-2-benzothiophen-4(5H)-one;
3-(Cyclopentyloxy)-6,6-dimethyl-1-(1H-pyrazol-3-yl)-6,7-dihydro-2-benzothiophen-4(5H)-one;
3-(Cyclobutyloxy)-6,6-dimethyl-1-(1H-pyrazol-3-yl)-6,7-dihydro-2-benzothiophen-4(5H)-one;
3-[3-(Cyclopentylsulfanyl)-6,6-dimethyl-4-oxo-4,5,6,7-tetrahydro-2-benzothiophen-1-yl]-N-[1-(trifluoroacetyl)piperidin-4-yl]-1H-pyrazole-1-carboxamide;
N-(3-Chlorophenyl)-3-[3-(cyclopentylsulfanyl)-6,6-dimethyl-4-oxo-4,5,6,7-tetrahydro-2-benzothiophen-1-yl]-1H-pyrazole-1-carboxamide;
N-(4-Chlorophenyl)-3-[3-(cyclopentylamino)-6,6-dimethyl-4-oxo-4,5,6,7-tetrahydro-2-benzothiophen-1-yl]-1H-pyrazole-1-carboxamide;
4-({3-[3-(Cyclopentylsulfanyl)-6,6-dimethyl-4-oxo-4,5,6,7-tetrahydro-2-benzothiophen-1-yl]-1H-pyrazol-1-yl}-carbonyl)benzonitrile;
3-(Cyclopentylsulfanyl)-1-{1-[(3,4-difluorophenyl)carbonyl]-1H-pyrazol-3-yl}-6,6-dimethyl-6,7-dihydro-2-benzothiophen-4(5H)-one;
N-(4-cyanophenyl)-3-[3-(cyclopentylamino)-4-oxo-4,5,6,7-tetrahydro-2-benzothiophen-1-yl]-1H-pyrazole-1-carboxamide;
1-(5-Cyclopropyl-1H-pyrazol-3-yl)-6,6-dimethyl-3-(methylsulfanyl)-6,7-dihydro-2-benzothiophen-4(5H)-one; or
1-(5-Ethyl-1H-pyrazol-3-yl)-6,6-dimethyl-3-(methylsulfanyl)-6,7-dihydro-2-benzothiophen-4(5H)-one.

13. A formulation comprising at least one of the following compounds or a salt thereof:
3-{[2-(Dimethylamino)ethyl]sulfanyl}-6,6-dimethyl-1-(1H-pyrazol-3-yl)-6,7-dihydro-2-benzothiophen-4(5H)-one;
6,6-Dimethyl-3-[(2-phenylethyl)sulfanyl]-1-(1H-pyrazol-3-yl)-6,7-dihydro-2-benzothiophen-4(5H)-one;
3-[(2,3-Dihydroxypropyl)sulfanyl]-6,6-dimethyl-1-(1H-pyrazol-3-yl)-6,7-dihydro-2-benzothiophen-4(5H)-one;
6,6-Dimethyl-3-(methylamino)-1-(1H-pyrazol-3-yl)-6,7-dihydro-2-benzothiophen-4(5H)-one;
3-(Azetidin-1-yl)-6,6-dimethyl-1-(1H-pyrazol-3-yl)-6,7-dihydro-2-benzothiophen-4(5H)-one;
6,6-Dimethyl-1-(1H-pyrazol-3-yl)-3-[(pyridin-2-ylmethyl)amino]-6,7-dihydro-2-benzothiophen-4(5H)-one;
6,6-Dimethyl-3-(propan-2-yloxy)-1-(1H-pyrazol-3-yl)-6,7-dihydro-2-benzothiophen-4(5H)-one;
N-benzyl-3-[3-(Cyclopentylsulfanyl)-6,6-dimethyl-4-oxo-4,5,6,7-tetrahydro-2-benzothiophen-1-yl]-1H-pyrazole-1-carboxamide;
3-[3-(Cyclopentylamino)-6,6-dimethyl-4-oxo-4,5,6,7-tetrahydro-2-benzothiophen-1-yl]-N-(pyridin-4-yl)-1H-pyrazole-1-carboxamide;
1-(1-Butanoyl-1H-pyrazol-3-yl)-3-(cyclopentylsulfanyl)-6,6-dimethyl-6,7-dihydro-2-benzothiophen-4(5H)-one;
1-{1-[(4-Chlorophenyl)acetyl]-1H-pyrazol-3-yl}-3-(cyclopentylsulfanyl)-6,6-dimethyl-6,7-dihydro-2-benzothiophen-4(5H)-one;
1-{1-[(6-Chloropyridin-3-yl)carbonyl]-1H-pyrazol-3-yl}-3-(cyclopentylsulfanyl)-6,6-dimethyl-6,7-dihydro-2-benzothiophen-4(5H)-one; or
3-(Cyclopentylsulfanyl)-1-{1-[(4-fluorophenyl)carbonyl]-1H-pyrazol-3-yl}-6,6-dimethyl-6,7-dihydro-2-benzothiophen-4(5H)-one.

14. A formulation comprising at least one of the following compounds or a salt thereof:
3-(Methylsulfonyl)-1-(1H-pyrazol-3-yl)-6,7-dihydro-2-benzothiophen-4(5H)-one;
6,6-Dimethyl-3-(methylsulfonyl)-1-(1H-pyrazol-3-yl)-6,7-dihydro-2-benzothiophen-4(5H)-one;
3-(Benzylsulfanyl)-6,6-dimethyl-1-(1H-pyrazol-3-yl)-6,7-dihydro-2-benzothiophen-4(5H)-one;
6,6-Dimethyl-1-(1H-pyrazol-3-yl)-3-{[2-(trimethylsilyl)ethyl]sulfanyl}-6,7-dihydro-2-benzothiophen-4(5H)-one;
6,6-Dimethyl-3-[(5-methyl-1,3,4-thiadiazol-2-yl)sulfanyl]-1-(1H-pyrazol-3-yl)-6,7-dihydro-2-benzothiophen-4(5H)-one;
3-(Hexylsulfanyl)-6,6-dimethyl-1-(1H-pyrazol-3-yl)-6,7-dihydro-2-benzothiophen-4(5H)-one;
3-(Dimethylamino)-6,6-dimethyl-1-(1H-pyrazol-3-yl)-6,7-dihydro-2-benzothiophen-4(5H)-one;
6,6-Dimethyl-3-(4-methylpiperazin-1-yl)-1-(1H-pyrazol-3-yl)-6,7-dihydro-2-benzothiophen-4(5H)-one;
6,6-Dimethyl-3-phenoxy-1-(1H-pyrazol-3-yl)-6,7-dihydro-2-benzothiophen-4(5H)-one;
6,6-Dimethyl-3-propyl-1-(1H-pyrazol-3-yl)-6,7-dihydro-2-benzothiophen-4(5H)-one;
3-[3-(Cyclopentylsulfanyl)-6,6-dimethyl-4-oxo-4,5,6,7-tetrahydro-2-benzothiophen-1-yl]-N-(propan-2-yl)-1H-pyrazole-1-carboxamide;
3-[3-(Cyclopentylsulfanyl)-6,6-dimethyl-4-oxo-4,5,6,7-tetrahydro-2-benzothiophen-1-yl]-N-(2-phenylethyl)-1H-pyrazole-1-carboxamide;
N-tert-Butyl-3-[3-(cyclopentylsulfanyl)-6,6-dimethyl-4-oxo-4,5,6,7-tetrahydro-2-benzothiophen-1-yl]-1H-pyrazole-1-carboxamide;
N-(Butan-2-yl)-3-[3-(cyclopentylsulfanyl)-6,6-dimethyl-4-oxo-4,5,6,7-tetrahydro-2-benzothiophen-1-yl]-1H-pyrazole-1-carboxamide;
3-[3-(Cyclopentylsulfanyl)-6,6-dimethyl-4-oxo-4,5,6,7-tetrahydro-2-benzothiophen-1-yl]-N-methyl-1H-pyrazole-1-carboxamide;
3-(Cyclopentylsulfanyl)-6,6-dimethyl-1-[1-(2-methylpropanoyl)-1H-pyrazol-3-yl]-6,7-dihydro-2-benzothiophen-4(5H)-one;
3-(Cyclopentylsulfanyl)-1-{1-[(4-methoxyphenyl)carbonyl]-1H-pyrazol-3-yl}-6,6-dimethyl-6,7-dihydro-2-benzothiophen-4(5H)-one;
1-[1-(Cyclohexylcarbonyl)-1H-pyrazol-3-yl]-3-(cyclopentylsulfanyl)-6,6-dimethyl-6,7-dihydro-2-benzothiophen-4(5H)-one;
tert-Butyl 3-[3-(methylsulfanyl)-4-oxo-4,5,6,7-tetrahydro-2-benzothiophen-1-yl]-1H-pyrazole-1-carboxylate; or
6,6-Dimethyl-1-(5-methyl-1H-pyrazol-3-yl)-3-(methylsulfanyl)-6,7-dihydro-2-benzothiophen-4(5H)-one.

15. The following compounds or a salt thereof:
3-(Cyclopentylsulfanyl)-6,6-dimethyl-1-(1H-pyrazol-3-yl)-6,7-dihydro-2-benzothiophen-4(5H)-one;
3-[(6-Hydroxyhexyl)sulfanyl]-6,6-dimethyl-1-(1H-pyrazol-3-yl)-6,7-dihydro-2-benzothiophen-4(5H)-one;
3-[(6-Aminohexyl)sulfanyl]-6,6-dimethyl-1-(1H-pyrazol-3-yl)-6,7-dihydro-2-benzothiophen-4(5H)-one;
3-(Cyclopentylamino)-6,6-dimethyl-1-(1H-pyrazol-3-yl)-6,7-dihydro-2-benzothiophen-4(5H)-one;
3-[3-(Cyclopentylsulfanyl)-6,6-dimethyl-4-oxo-4,5,6,7-tetrahydro-2-benzothiophen-1-yl]-N-[4-(trifluoromethyl)phenyl]-1H-pyrazole-1-carboxamide;
N-(4-Chlorophenyl)-3-[3-(cyclopentylsulfanyl)-6,6-dimethyl-4-oxo-4,5,6,7-tetrahydro-2-benzothiophen-1-yl]-1H-pyrazole-1-carboxamide;
N-(4-Cyanophenyl)-3-[3-(cyclopentylsulfanyl)-6,6-dimethyl-4-oxo-4,5,6,7-tetrahydro-2-benzothiophen-1-yl]-1H-pyrazole-1-carboxamide; or
N-(4-chlorophenyl)-3-[3-(cyclopentylsulfanyl)-4-oxo-4,5,6,7-tetrahydro-2-benzothiophen-1-yl]-1H-pyrazole-1-carboxamide.

16. The following compounds or a salt thereof:
3-[(Furan-2-ylmethyl)sulfanyl]-6,6-dimethyl-1-(1H-pyrazol-3-yl)-6,7-dihydro-2-benzothiophen-4(5H)-one;
3-[(6-Azidohexyl)sulfanyl]-6,6-dimethyl-1-(1H-pyrazol-3-yl)-6,7-dihydro-2-benzothiophen-4(5H)-one;
6,6-Dimethyl-3-{[3-(4-methylpiperazin-1-yl)propyl]amino}-1-(1H-pyrazol-3-yl)-6,7-dihydro-2-benzothiophen-4(5H)-one;
3-[(Furan-2-ylmethyl)amino]-6,6-dimethyl-1-(1H-pyrazol-3-yl)-6,7-dihydro-2-benzothiophen-4(5H)-one;
6,6-Dimethyl-3-{[2-(morpholin-4-yl)ethyl]amino}-1-(1H-pyrazol-3-yl)-6,7-dihydro-2-benzothiophen-4(5H)-one;
6,6-Dimethyl-1-(1H-pyrazol-3-yl)-3-{[2-(pyridin-2-yl)ethyl]amino}-6,7-dihydro-2-benzothiophen-4(5H)-one;
3-(Cyclopentyloxy)-6,6-dimethyl-1-(1H-pyrazol-3-yl)-6,7-dihydro-2-benzothiophen-4(5H)-one;
3-(Cyclobutyloxy)-6,6-dimethyl-1-(1H-pyrazol-3-yl)-6,7-dihydro-2-benzothiophen-4(5H)-one;
3-[3-(Cyclopentylsulfanyl)-6,6-dimethyl-4-oxo-4,5,6,7-tetrahydro-2-benzothiophen-1-yl]-N-[1-(trifluoroacetyl)piperidin-4-yl]-1H-pyrazole-1-carboxamide;
N-(3-Chlorophenyl)-3-[3-(cyclopentylsulfanyl)-6,6-dimethyl-4-oxo-4,5,6,7-tetrahydro-2-benzothiophen-1-yl]-1H-pyrazole-1-carboxamide;
N-(4-Chlorophenyl)-3-[3-(cyclopentylamino)-6,6-dimethyl-4-oxo-4,5,6,7-tetrahydro-2-benzothiophen-1-yl]-1H-pyrazole-1-carboxamide;
4-({3-[3-(Cyclopentylsulfanyl)-6,6-dimethyl-4-oxo-4,5,6,7-tetrahydro-2-benzothiophen-1-yl]-1H-pyrazol-1-yl}carbonyl)benzonitrile;
3-(Cyclopentylsulfanyl)-1-{1-[(3,4-difluorophenyl)carbonyl]-1H-pyrazol-3-yl}-6,6-dimethyl-6,7-dihydro-2-benzothiophen-4(5H)-one;
N-(4-cyanophenyl)-3-[3-(cyclopentylamino)-4-oxo-4,5,6,7-tetrahydro-2-benzothiophen-1-yl]-1H-pyrazole-1-carboxamide;
1-(5-Cyclopropyl-1H-pyrazol-3-yl)-6,6-dimethyl-3-(methylsulfanyl)-6,7-dihydro-2-benzothiophen-4(5H)-one; or
1-(5-Ethyl-1H-pyrazol-3-yl)-6,6-dimethyl-3-(methylsulfanyl)-6,7-dihydro-2-benzothiophen-4(5H)-one.

17. A formulation comprising at least one of the following compounds or a salt thereof:
3-{[2-(Dimethylamino)ethyl]sulfanyl}-6,6-dimethyl-1-(1H-pyrazol-3-yl)-6,7-dihydro-2-benzothiophen-4(5H)-one;
3-(Azetidin-1-yl)-6,6-dimethyl-1-(1H-pyrazol-3-yl)-6,7-dihydro-2-benzothiophen-4(5H)-one;
6,6-Dimethyl-1-(1H-pyrazol-3-yl)-3-[(pyridin-2-ylmethyl)amino]-6,7-dihydro-2-benzothiophen-4(5H)-one;
N-benzyl-3-[3-(Cyclopentylsulfanyl)-6,6-dimethyl-4-oxo-4,5,6,7-tetrahydro-2-benzothiophen-1-yl]-1H-pyrazole-1-carboxamide;
3-[3-(Cyclopentylamino)-6,6-dimethyl-4-oxo-4,5,6,7-tetrahydro-2-benzothiophen-1-yl]-N-(pyridin-4-yl)-1H-pyrazole-1-carboxamide;
1-(1-Butanoyl-1H-pyrazol-3-yl)-3-(cyclopentylsulfanyl)-6,6-dimethyl-6,7-dihydro-2-benzothiophen-4(5H)-one;
1-{1-[(4-Chlorophenyl)acetyl]-1H-pyrazol-3-yl}-3-(cyclopentylsulfanyl)-6,6-dimethyl-6,7-dihydro-2-benzothiophen-4(5H)-one;
1-{1-[(6-Chloropyridin-3-yl)carbonyl]-1H-pyrazol-3-yl}-3-(cyclopentylsulfanyl)-6,6-dimethyl-6,7-dihydro-2-benzothiophen-4(5H)-one; or
3-(Cyclopentylsulfanyl)-1-{1-[(4-fluorophenyl)carbonyl]-1H-pyrazol-3-yl}-6,6-dimethyl-6,7-dihydro-2-benzothiophen-4(5H)-one.

18. A method of treating, managing, or ameliorating diseases amenable to treatment, management, or amelioration by inhibition of LRRK2 kinase activity comprising administration of a therapeutically effective amount of a formulation of claim 1 to a patient afflicted with a disease which can be treated, managed, or ameliorated by inhibition of LRRK2 kinase activity.

19. A method of treating, managing, or ameliorating diseases amenable to treatment, management, or amelioration by inhibition of LRRK2 kinase activity comprising administration of a therapeutically effective amount of a compound of claim 2 to a patient afflicted with a disease which can be treated, managed, or ameliorated by inhibition of LRRK2 kinase activity.

20. The method of either claim 19 or claim 20, wherein said disease is Parkinson's disease or non-skin cancers associated with mutant LRRK2 function.

21. A method of treating, managing, or ameliorating diseases amenable to treatment, management, or amelioration by inhibition of LRRK2 kinase activity comprising administration of a therapeutically effective amount of a compound of Formula I as the compound of Formula I is defined in claim 1.
